SEQUENCE LISTING (217 sequences)

SEQ ID NO: 1 - protein, 64 aa, Monkeypox virus
MYYANICLDFDNNVYTVKDKNYTNAVIEYPVVCNFRRYSESDSDVDDRAELHKRNNDSDSDDYT

SEQ ID NO: 2 - protein, 20 aa, Monkeypox virus
MYYANICLDFDNNVYTVKDK

SEQ ID NO: 3 - protein, 20 aa, Monkeypox virus
DNNVYTVKDKNYTNAVIEYP

SEQ ID NO: 4 - protein, 20 aa, Monkeypox virus
NYTNAVIEYPVVCNFRRYSE

SEQ ID NO: 5 - protein, 20 aa, Monkeypox virus
VVCNFRRYSESDSDVDDRAE

SEQ ID NO: 6 - protein, 73 aa, Monkeypox virus
MQYLNETDNLGNTVLHTHIFLDYISLKICKRYISHKYPLCNIINGYIDNTIGTNSIVKDIIDYLYISRYLYSY

SEQ ID NO: 7 - protein, 20 aa, Monkeypox virus
MQYLNETDNLGNTVLHTHIF

SEQ ID NO: 8 - protein, 20 aa, Monkeypox virus
LDYISLKICKRYISHKYPLC

SEQ ID NO: 9 - protein, 20 aa, Monkeypox virus
RYISHKYPLCNIINGYIDNT

SEQ ID NO: 10 - protein, 176 aa, Monkeypox virus
MDSRIAIYVLVSASLLYLVNCHKLVHYFNLKINGSDITNTADILLDNYPIMTFDGKDIYPSIAFMVGNKLFLDLYKNIFVEFFRLFRVSVSSQYEELEYYYSCDYTNNRPTIKQHYFYNGEEYTEIDRSKKATNKNSWLITSGFRLQKWFDSEDCIIYLRSLVRRMEDSNKNSKKT

SEQ ID NO: 11 - protein, 20 aa, Monkeypox virus
CHKLVHYFNLKINGSDITNT

SEQ ID NO: 12 - protein, 20 aa, Monkeypox virus
SSQYEELEYYYSCDYTNNRP

SEQ ID NO: 13 - protein, 20 aa, Monkeypox virus
TIKQHYFYNGEEYTEIDRSK

SEQ ID NO: 14 - protein, 20 aa, Monkeypox virus
DSEDCIIYLRSLVRRMEDSN

SEQ ID NO: 15 - protein, 20 aa, Monkeypox virus
IYLRSLVRRMEDSNKNSKKT

SEQ ID NO: 16 - protein, 70 aa, Monkeypox virus
MIDILLCDVIITIGDVEIKAHKTILAAGSTYFKTMFTTPMIARDLATRVNLQMFDKMPSKILYSTYTIGI

SEQ ID NO: 17 - protein, 20 aa, Monkeypox virus
YFKTMFTTPMIARDLATRVN

SEQ ID NO: 18 - protein, 20 aa, Monkeypox virus
IARDLATRVNLQMFDKMPSK

SEQ ID NO: 19 - protein, 20 aa, Monkeypox virus
LQMFDKMPSKILYSTYTIGI

SEQ ID NO: 20 - protein, 1879 aa, Monkeypox virus
MNLQKLSLAIYLTVTCSWCYETCMRKTALYHDIQLEHVEDNKDSVASLPYKYLQVVKQRERSRLLATFNWTDIAEGVRNEFIKICDINGTYLYNYTIDVSIIIDSTEELPTVTPITTYEPSIYNYTIDYSTVITTEELQVTPTYAPVTTPLPTSAVPYDQRSNNNVSTISIQILSKILGVNETELTNYLIMHKNDTVDNNTMVDDETSDNNTLHGNIGFLEINNCYNVSVSDASFRITLVNDTSEEILLMLTGTSSSDTFISSTNITECLKTLINNVSINDVLITQNMNVTSNCDKCSMNLMASVIPAVNEFNNTLMKIGVKDDENNTVYNYYICKLTTNSTCDELINLDEVINNITLTNIIRNSVSTTNSRKRRDLNGEFEFSTSKELDCLYESYGVNDDISHCFASPRRRRSDDKKEYMDMKLFDHAKKDLGIDSVIPRGTTHFQVGASGASGGVVGDSFPFQNVKSRASLLAEKIMPRVPITATEADLYATVNRQPKLPAGVKSTPFTEALASTINQKLSNVREVTYASSNLPGSSGYVHRPSDSVIYSSIRRSRLPSDSDSDYEDIQTVVKEYNERYGRSVSRTQSSSSESDFEDIDTVVREYRQKYGNAMAKGRSSSPKPDPLYSTVKKTTKSLSTGVDIVTKQSDYSLLPDVNTGSSIVSPLTRKGATRRRPRRPTNDGLQSPNPPLRNPLPQHDDYSPPQVHRPPTLPPKPVQNPTQLPPRPVGQLPPPIDQPDKGFSKFVSPRRCRRASSGVICGMIQSKPNDDTYSLLQRPKIEPEYAEVGNGIPKNNVPVIGNKHSKKYTSTMSKISTKFDKSTAFGAAMLLTGQQAISQQTRSTTLSRKDQMSKEEKIFEAVTMSLSTIGSTLTSAGMTGGPKLMIAGMAITAITGIIDTIKDIYYMFSGQERPVDPVIKLFNKYAGLMSDNNKMGVRKCLTPGDDTLIYIAYRNDTSFKQNTDAMALYFLDVIDSEILYLNTSNLVLEYQLKVACPIGTLRSVDVDITAYTILYDTADNIKKYKFIRMATLLSKHPVIRLTCGLAATLVIKPYEVPISDMQLLKMATPGEPESTKSIPSDVCDRYPLKKFYLLAGGCPYDTSQTFIVHTTCSILLRTATRDQFRNRWVLQNPFRQEGTYKQLFTFSKYDFNDTIIDPNGVVGHASFCTNRSSNQCFWSEPMILEDVSSCSSRTRKIYVKLGIFNAEGFNSFVLNCPTGSTPTYIKHKNADSNNVIIELPVGDYGTAKLYSATKPSRIAVFCTHNYDKRFKSDIIVLMFNKNSGIPFWSMYTGSVTSKNRMFTTLARGMPFRSTYCDNRRRSGCYYAGIPFHEDSVEADIHYGPEIMLKETYDINSIDPRVITKSKTHFPTPLSVKFMVDNLGNGYDNPNSFWEDAKTKKRTYSAMTIKVLPCTVRNKNIDFGYNYGDIISNMVYLQSTSQDYGDGTKYTFKSVTRSDHECESSLDLTSKEVTVTCPAFSIPRNISTYEGLCFSVTTSKDHCATGIGWLKSSGYGKEDADKPRACFHHWNYYTLSLDYYCSYEDIWRSTWPDYDPCKSYIHIEYRDTWIESNVLQQPPYTFEFIHDNSNEYVDKEISNKLNDLYNEYKKIMEYSDGSLPASINRLAKALTSEGREIASVNIDGNLLDIAYQADKEKMADIQTRINDIIRDLFIHTLSDKDIKDIIESEEGKRCCIIDVKNNRVKKYYSIDNYLCGTLDDYIYTVVEYNKSYVLVNDTYMSYDYLESSGVVVLSCYEMTIISLDTKDAKDAIEDVIVASAVAEALNDMFKEFDKNVSAIIIKEEDNYLNSSPDIYHIIYIIGGTILLLLVIILILAIYIARNKYRTRKYEIMKYDNMSIKSDHHDSLETVSMEIIDNRY

SEQ ID NO: 21 - protein, 20 aa, Monkeypox virus
PLPTSAVPYDQRSNNNVSTI

SEQ ID NO: 22 - protein, 20 aa, Monkeypox virus
DTVDNNTMVDDETSDNNTLH

SEQ ID NO: 23 - protein, 20 aa, Monkeypox virus
IRNSVSTTNSRKRRDLNGEF

SEQ ID NO: 24 - protein, 20 aa, Monkeypox virus
TRKGATRRRPRRPTNDGLQS

SEQ ID NO: 25 - protein, 20 aa, Monkeypox virus
DGLQSPNPPLRNPLPQHDDY

SEQ ID NO: 26 - protein, 20 aa, Monkeypox virus
QHDDYSPPQVHRPPTLPPKP

SEQ ID NO: 27 - protein, 20 aa, Monkeypox virus
PVGQLPPPIDQPDKGFSKFV

SEQ ID NO: 28 - protein, 20 aa, Monkeypox virus
KNNVPVIGNKHSKKYTSTMS

SEQ ID NO: 29 - protein, 20 aa, Monkeypox virus
TRSTTLSRKDQMSKEEKIFE

SEQ ID NO: 30 - protein, 20 aa, Artificial sequence
  Monkeypox virus B21R polypeptide 42
RRRSDDKKEYMDMKLFDHAK

SEQ ID NO: 31 - protein, 20 aa, Artificial sequence
  Monkeypox virus B21R polypeptide 67
GSSIVSPLTRKGATRRRPRR

SEQ ID NO: 32 - protein, 20 aa, Artificial sequence
  Monkeypox virus B21R polypeptide 73
NPTQLPPRPVGQLPPPIDQP

SEQ ID NO: 33 - protein, 20 aa, Artificial sequence
  Monkeypox virus B21R polypeptide 75
DKGFSKFVSPRRCRRASSGV

SEQ ID NO: 34 - protein, 20 aa, Artificial sequence
  Monkeypox virus B21R polypeptide 100
YQLKVACPIGTLRSVDVDIT

SEQ ID NO: 35 - protein, 20 aa, Artificial sequence
  Monkeypox virus B21R polypeptide 132
PFRSTYCDNRRRSGCYYAGI

SEQ ID NO: 36 - protein, 20 aa, Artificial sequence
  Monkeypox virus B21R polypeptide 141
KRTYSAMTIKVLPCTVRNKN

SEQ ID NO: 37 - protein, 20 aa, Artificial sequence
  Monkeypox virus B21R polypeptide 152
KSSGYGKEDADKPRACFHHW

SEQ ID NO: 38 - protein, 20 aa, Artificial sequence
  Monkeypox virus B21R polypeptide 159
TFEFIHDNSNEYVDKEISNK

SEQ ID NO: 39 - protein, 20 aa, Artificial sequence
  Monkeypox virus B21R polypeptide 166
YQADKEKMADIQTRINDIIR

SEQ ID NO: 40 - protein, 20 aa, Artificial sequence
  Monkeypox virus B21R polypeptide 168
DLFIHTLSDKDIKDIIESEE

SEQ ID NO: 41 - protein, 20 aa, Artificial sequence
  Monkeypox virus B21R polypeptide 170
GKRCCIIDVKNNRVKKYYSI

SEQ ID NO: 42 - protein, 20 aa, Artificial sequence
  Monkeypox virus B21R polypeptide 172
DNYLCGTLDDYIYTVVEYNK

SEQ ID NO: 43 - protein, 20 aa, Artificial sequence
  Monkeypox virus B21R polypeptide 178
AIEDVIVASAVAEALNDMFK

SEQ ID NO: 44 - protein, 20 aa, Artificial sequence
  Monkeypox virus B21R polypeptide 184
IILILAIYIARNKYRTRKYE

SEQ ID NO: 45 - protein, 20 aa, Artificial sequence
  Monkeypox virus BR21R polypeptide 1
MNLQKLSLAIYLTVTCSWCY

SEQ ID NO: 46 - protein, 20 aa, Artificial sequence
  Monkeypox virus B21R polypeptide 2
YLTVTCSWCYETCMRKTALY

SEQ ID NO: 47 - protein, 20 aa, Artificial sequence
  Monkeypox virus B21R polypeptide 3
ETCMRKTALYHDIQLEHVED

SEQ ID NO: 48 - protein, 20 aa, Artificial sequence
  Monkeypox virus B21R polypeptide 4
HDIQLEHVEDNKDSVASLPY

SEQ ID NO: 49 - protein, 20 aa, Artificial sequence
  Monkeypox virus B21R polypeptide 5
NKDSVASLPYKYLQVVKQRE

SEQ ID NO: 50 - protein, 20 aa, Artificial sequence
  Monkeypox virus B21R polypeptide 6
KYLQVVKQRERSRLLATFNW

SEQ ID NO: 51 - protein, 20 aa, Artificial sequence
  Monkeypox virus B21R polypeptide 7
RSRLLATFNWTDIAEGVRNE

SEQ ID NO: 52 - protein, 20 aa, Artificial sequence
  Monkeypox virus B21R polypeptide 8
TDIAEGVRNEFIKICDINGT

SEQ ID NO: 53 - protein, 20 aa, Artificial sequence
  Monkeypox virus B21R polypeptide 9
FIKICDINGTYLYNYTIDVS

SEQ ID NO: 54 - protein, 20 aa, Artificial sequence
  Monkeypox virus B21R polypeptide 10
YLYNYTIDVSIIIDSTEELP

SEQ ID NO: 55 - protein, 20 aa, Artificial sequence
  Monkeypox virus B21R polypeptide 11
IIIDSTEELPTVTPITTYEP

SEQ ID NO: 56 - protein, 20 aa, Artificial sequence
  Monkeypox virus B21R polypeptide 12
TVTPITTYEPSIYNYTIDYS

SEQ ID NO: 57 - protein, 20 aa, Artificial sequence
  Monkeypox virus B21R polypeptide 13
SIYNYTIDYSTVITTEELQV

SEQ ID NO: 58 - protein, 20 aa, Artificial sequence
  Monkeypox virus B21R polypeptide 14
TVITTEELQVTPTYAPVTTP

SEQ ID NO: 59 - protein, 20 aa, Artificial sequence
  Monkeypox virus B21R polypeptide 15
TPTYAPVTTPLPTSAVPYDQ

SEQ ID NO: 60 - protein, 20 aa, Artificial sequence
  Monkeypox virus B21R polypeptide 16
LPTSAVPYDQRSNNNVSTIS

SEQ ID NO: 61 - protein, 20 aa, Artificial sequence
  Monkeypox virus B21R polypeptide 17
RSNNNVSTISIQILSKILGV

SEQ ID NO: 62 - protein, 20 aa, Artificial sequence
  Monkeypox virus B21R polypeptide 18
IQILSKILGVNETELTNYLI

SEQ ID NO: 63 - protein, 20 aa, Artificial sequence
  Monkeypox virus B21R polypeptide 19
NETELTNYLIMHKNDTVDNN

SEQ ID NO: 64 - protein, 20 aa, Artificial sequence
  Monkeypox virus B21R polypeptide 20
MHKNDTVDNNTMVDDETSDN

SEQ ID NO: 65 - protein, 20 aa, Artificial sequence
  Monkeypox virus B21R polypeptide 21
TMVDDETSDNNTLHGNIGFL

SEQ ID NO: 66 - protein, 20 aa, Artificial sequence
  Monkeypox virus B21R polypeptide 22
NTLHGNIGFLEINNCYNVSV

SEQ ID NO: 67 - protein, 20 aa, Artificial sequence
  Monkeypox virus B21R polypeptide 23
EINNCYNVSVSDASFRITLV

SEQ ID NO: 68 - protein, 20 aa, Artificial sequence
  Monkeypox virus B21R polypeptide 24
SDASFRITLVNDTSEEILLM

SEQ ID NO: 69 - protein, 20 aa, Artificial sequence
  Monkeypox virus B21R polypeptide 25
NDTSEEILLMLTGTSSSDTF

SEQ ID NO: 70 - protein, 20 aa, Artificial sequence
  Monkeypox virus B21R polypeptide 26
LTGTSSSDTFISSTNITECL

SEQ ID NO: 71 - protein, 20 aa, Artificial sequence
  Monkeypox virus B21R polypeptide 27
ISSTNITECLKTLINNVSIN

SEQ ID NO: 72 - protein, 20 aa, Artificial sequence
  Monkeypox virus B21R polypeptide 28
KTLINNVSINDVLITQNMNV

SEQ ID NO: 73 - protein, 20 aa, Artificial sequence
  Monkeypox virus B21R polypeptide 29
DVLITQNMNVTSNCDKCSMN

SEQ ID NO: 74 - protein, 20 aa, Artificial sequence
  Monkeypox virus B21R polypeptide 30
TSNCDKCSMNLMASVIPAVN

SEQ ID NO: 75 - protein, 20 aa, Artificial sequence
  Monkeypox virus B21R polypeptide 31
LMASVIPAVNEFNNTLMKIG

SEQ ID NO: 76 - protein, 20 aa, Artificial sequence
  Monkeypox virus B21R polypeptide 32
EFNNTLMKIGVKDDENNTVY

SEQ ID NO: 77 - protein, 20 aa, Artificial sequence
  Monkeypox virus B21R polypeptide 33
VKDDENNTVYNYYICKLTTN

SEQ ID NO: 78 - protein, 20 aa, Artificial sequence
  Monkeypox virus B21R polypeptide 34
NYYICKLTTNSTCDELINLD

SEQ ID NO: 79 - protein, 20 aa, Artificial sequence
  Monkeypox virus B21R polypeptide 35
STCDELINLDEVINNITLTN

SEQ ID NO: 80 - protein, 20 aa, Artificial sequence
  Monkeypox virus B21R polypeptide 36
EVINNITLTNIIRNSVSTTN

SEQ ID NO: 81 - protein, 20 aa, Artificial sequence
  Monkeypox virus B21R polypeptide 37
IIRNSVSTTNSRKRRDLNGE

SEQ ID NO: 82 - protein, 20 aa, Artificial sequence
  Monkeypox virus B21R polypeptide 38
SRKRRDLNGEFEFSTSKELD

SEQ ID NO: 83 - protein, 20 aa, Artificial sequence
  Monkeypox virus B21R polypeptide 39
FEFSTSKELDCLYESYGVND

SEQ ID NO: 84 - protein, 20 aa, Artificial sequence
  Monkeypox virus B21R polypeptide 40
CLYESYGVNDDISHCFASPR

SEQ ID NO: 85 - protein, 20 aa, Artificial sequence
  Monkeypox virus B21R polypeptide 41
DISHCFASPRRRRSDDKKEY

SEQ ID NO: 86 - protein, 20 aa, Artificial sequence
  Monkeypox virus B21R polypeptide 43
MDMKLFDHAKKDLGIDSVIP

SEQ ID NO: 87 - protein, 20 aa, Artificial sequence
  Monkeypox virus B21R polypeptide 44
KDLGIDSVIPRGTTHFQVGA

SEQ ID NO: 88 - protein, 20 aa, Artificial sequence
  Monkeypox virus B21R polypeptide 45
RGTTHFQVGASGASGGVVGD

SEQ ID NO: 89 - protein, 20 aa, Artificial sequence
  Monkeypox virus B21R polypeptide 46
SGASGGVVGDSFPFQNVKSR

SEQ ID NO: 90 - protein, 20 aa, Artificial sequence
  monkeypox virus B21R polypeptide 47
SFPFQNVKSRASLLAEKIMP

SEQ ID NO: 91 - protein, 20 aa, Artificial sequence
  Monkeypox virus B21R polypeptide 48
ASLLAEKIMPRVPITATEAD

SEQ ID NO: 92 - protein, 20 aa, Artificial sequence
  Monkeypox virus B21R polypeptide 49
RVPITATEADLYATVNRQPK

SEQ ID NO: 93 - protein, 20 aa, Artificial sequence
  Monkeypox virus B21R polypeptide 50
LYATVNRQPKLPAGVKSTPF

SEQ ID NO: 94 - protein, 20 aa, Artificial sequence
  Monkeypox virus B21R polypeptide 51
LPAGVKSTPFTEALASTINQ

SEQ ID NO: 95 - protein, 20 aa, Artificial sequence
  Monkeypox virus B21R polypeptide 52
TEALASTINQKLSNVREVTY

SEQ ID NO: 96 - protein, 20 aa, Artificial sequence
  Monkeypox virus B21R polypeptide 53
KLSNVREVTYASSNLPGSSG

SEQ ID NO: 97 - protein, 20 aa, Artificial sequence
  Monkeypox virus B21R polypeptide 54
ASSNLPGSSGYVHRPSDSVI

SEQ ID NO: 98 - protein, 20 aa, Artificial sequence
  Monkeypox virus B21R polypeptide 55
YVHRPSDSVIYSSIRRSRLP

SEQ ID NO: 99 - protein, 20 aa, Artificial sequence
  Monkeypox virus B21R polypeptide 56
YSSIRRSRLPSDSDSDYEDI

SEQ ID NO: 100 - protein, 20 aa, Artificial sequence
  Monkeypox virus B21R polypeptide 57
SDSDSDYEDIQTVVKEYNER

SEQ ID NO: 101 - protein, 20 aa, Artificial sequence
  Monkeypox virus B21R polypeptide 58
QTVVKEYNERYGRSVSRTQS

SEQ ID NO: 102 - protein, 20 aa, Artificial sequence
  Monkeypox virus B21R polypeptide 59
YGRSVSRTQSSSSESDFEDI

SEQ ID NO: 103 - protein, 20 aa, Artificial sequence
  Monkeypox virus B21R polypeptide 60
SSSESDFEDIDTVVREYRQK

SEQ ID NO: 104 - protein, 20 aa, Artificial sequence
  Monkeypox virus B21R polypeptide 61
DTVVREYRQKYGNAMAKGRS

SEQ ID NO: 105 - protein, 20 aa, Artificial sequence
  Monkeypox virus B21R polypeptide 62
YGNAMAKGRSSSPKPDPLYS

SEQ ID NO: 106 - protein, 20 aa, Artificial sequence
  Monkeypox virus B21R polypeptide 63
SSPKPDPLYSTVKKTTKSLS

SEQ ID NO: 107 - protein, 20 aa, Artificial sequence
  Monkeypox virus B21R polypeptide 64
TVKKTTKSLSTGVDIVTKQS

SEQ ID NO: 108 - protein, 20 aa, Artificial sequence
  Monkeypox virus B21R polypeptide 65
TGVDIVTKQSDYSLLPDVNT

SEQ ID NO: 109 - protein, 20 aa, Artificial sequence
  Monkeypox virus B21R polypeptide 66
DYSLLPDVNTGSSIVSPLTR

SEQ ID NO: 110 - protein, 20 aa, Artificial sequence
  Monkeypox virus B21R polypeptide 68
KGATRRRPRRPTNDGLQSPN

SEQ ID NO: 111 - protein, 20 aa, Artificial sequence
  Monkeypox virus B21R polypeptide 69
PTNDGLQSPNPPLRNPLPQH

SEQ ID NO: 112 - protein, 20 aa, Artificial sequence
  Monkeypox virus B21R polypeptide 70
PPLRNPLPQHDDYSPPQVHR

SEQ ID NO: 113 - protein, 20 aa, Artificial sequence
  Monkeypox virus B21R polypeptide 71
DDYSPPQVHRPPTLPPKPVQ

SEQ ID NO: 114 - protein, 20 aa, Artificial sequence
  Monkeypox virus B21R polypeptide 72
PPTLPPKPVQNPTQLPPRPV

SEQ ID NO: 115 - protein, 20 aa, Artificial sequence
  Monkeypox virus B21R polypeptide 74
GQLPPPIDQPDKGFSKFVSP

SEQ ID NO: 116 - protein, 20 aa, Artificial sequence
  Monkeypox virus B21R polypeptide 76
RRCRRASSGVICGMIQSKPN

SEQ ID NO: 117 - protein, 20 aa, Artificial sequence
  Monkeypox virus B21R polypeptide 77
ICGMIQSKPNDDTYSLLQRP

SEQ ID NO: 118 - protein, 20 aa, Artificial sequence
  Monkeypox virus B21R polypeptide 78
DDTYSLLQRPKIEPEYAEVG

SEQ ID NO: 119 - protein, 20 aa, Artificial sequence
  Monkeypox virus B21R polypeptide 79
KIEPEYAEVGNGIPKNNVPV

SEQ ID NO: 120 - protein, 20 aa, Artificial sequence
  Monkeypox virus B21R polypeptide 80
NGIPKNNVPVIGNKHSKKYT

SEQ ID NO: 121 - protein, 20 aa, Artificial sequence
  Monkeypox virus B21R polypeptide 81
IGNKHSKKYTSTMSKISTKF

SEQ ID NO: 122 - protein, 20 aa, Artificial sequence
  Monkeypox virus B21R polypeptide 82
STMSKISTKFDKSTAFGAAM

SEQ ID NO: 123 - protein, 20 aa, Artificial sequence
  Monkeypox virus B21R polypeptide 83
DKSTAFGAAMLLTGQQAISQ

SEQ ID NO: 124 - protein, 20 aa, Artificial sequence
  Monkeypox virus B21R polypeptide 84
LLTGQQAISQQTRSTTLSRK

SEQ ID NO: 125 - protein, 20 aa, Artificial sequence
  Monkeypox virus B21R polypeptide 85
QTRSTTLSRKDQMSKEEKIF

SEQ ID NO: 126 - protein, 20 aa, Artificial sequence
  Monkeypox virus B21R polypeptide 86
DQMSKEEKIFEAVTMSLSTI

SEQ ID NO: 127 - protein, 20 aa, Artificial sequence
  Monkeypox virus B21R polypeptide 87
EAVTMSLSTIGSTLTSAGMT

SEQ ID NO: 128 - protein, 20 aa, Artificial sequence
  Monkeypox virus B21R polypeptide 88
GSTLTSAGMTGGPKLMIAGM

SEQ ID NO: 129 - protein, 20 aa, Artificial sequence
  Monkeypox virus B21R polypeptide 89
GGPKLMIAGMAITAITGIID

SEQ ID NO: 130 - protein, 20 aa, Artificial sequence
  Monkeypox virus B21R polypeptide 90
AITAITGIIDTIKDIYYMFS

SEQ ID NO: 131 - protein, 20 aa, Artificial sequence
  Monkeypox virus B21R polypeptide 91
TIKDIYYMFSGQERPVDPVI

SEQ ID NO: 132 - protein, 20 aa, Artificial sequence
  Monkeypox virus B21R polypeptide 92
GQERPVDPVIKLFNKYAGLM

SEQ ID NO: 133 - protein, 20 aa, Artificial sequence
  Monkeypox virus B21R polypeptide 93
KLFNKYAGLMSDNNKMGVRK

SEQ ID NO: 134 - protein, 20 aa, Artificial sequence
  Monkeypox virus B21R polypeptide 94
SDNNKMGVRKCLTPGDDTLI

SEQ ID NO: 135 - protein, 20 aa, Artificial sequence
  Monkeypox virus B21R polypeptide 95
CLTPGDDTLIYIAYRNDTSF

SEQ ID NO: 136 - protein, 20 aa, Artificial sequence
  Monkeypox virus B21R polypeptide 96
YIAYRNDTSFKQNTDAMALY

SEQ ID NO: 137 - protein, 20 aa, Artificial sequence
  Monkeypox virus B21R polypeptide 97
KQNTDAMALYFLDVIDSEIL

SEQ ID NO: 138 - protein, 20 aa, Artificial sequence
  Monkeypox virus B21R polypeptide 98
FLDVIDSEILYLNTSNLVLE

SEQ ID NO: 139 - protein, 20 aa, Artificial sequence
  Monkeypox virus B21R polypeptide 99
YLNTSNLVLEYQLKVACPIG

SEQ ID NO: 140 - protein, 20 aa, Artificial sequence
  Monkeypox virus B21R polypeptide 101
TLRSVDVDITAYTILYDTAD

SEQ ID NO: 141 - protein, 20 aa, Artificial sequence
  Monkeypox virus B21R polypeptide 102
AYTILYDTADNIKKYKFIRM

SEQ ID NO: 142 - protein, 20 aa, Artificial sequence
  Monkeypox virus B21R polypeptide 103
NIKKYKFIRMATLLSKHPVI

SEQ ID NO: 143 - protein, 20 aa, Artificial sequence
  Monkeypox virus B21R polypeptide 104
ATLLSKHPVIRLTCGLAATL

SEQ ID NO: 144 - protein, 20 aa, Artificial sequence
  Monkeypox virus B21R polypeptide 105
RLTCGLAATLVIKPYEVPIS

SEQ ID NO: 145 - protein, 20 aa, Artificial sequence
  Monkeypox virus B21R polypeptide 106
VIKPYEVPISDMQLLKMATP

SEQ ID NO: 146 - protein, 20 aa, Artificial sequence
  Monkeypox virus B21R polypeptide 107
DMQLLKMATPGEPESTKSIP

SEQ ID NO: 147 - protein, 20 aa, Artificial sequence
  Monkeypox virus B21R polypeptide 108
GEPESTKSIPSDVCDRYPLK

SEQ ID NO: 148 - protein, 20 aa, Artificial sequence
  Monkeypox virus B21R polypeptide 109
SDVCDRYPLKKFYLLAGGCP

SEQ ID NO: 149 - protein, 20 aa, Artificial sequence
  Monkeypox virus B21R polypeptide 110
KFYLLAGGCPYDTSQTFIVH

SEQ ID NO: 150 - protein, 20 aa, Artificial sequence
  Monkeypox virus B21R polypeptide 111
YDTSQTFIVHTTCSILLRTA

SEQ ID NO: 151 - protein, 20 aa, Artificial sequence
  Monkeypox virus B21R polypeptide 112
TTCSILLRTATRDQFRNRWV

SEQ ID NO: 152 - protein, 20 aa, Artificial sequence
  Monkeypox virus B21R polypeptide 113
TRDQFRNRWVLQNPFRQEGT

SEQ ID NO: 153 - protein, 20 aa, Artificial sequence
  Monkeypox virus B21R polypeptide 114
LQNPFRQEGTYKQLFTFSKY

SEQ ID NO: 154 - protein, 20 aa, Artificial sequence
  Monkeypox virus B21R polypeptide 115
YKQLFTFSKYDFNDTIIDPN

SEQ ID NO: 155 - protein, 20 aa, Artificial sequence
  Monkeypox virus B21R polypeptide 116
DFNDTIIDPNGVVGHASFCT

SEQ ID NO: 156 - protein, 20 aa, Artificial sequence
  Monkeypox virus B21R polypeptide 117
GVVGHASFCTNRSSNQCFWS

SEQ ID NO: 157 - protein, 20 aa, Artificial sequence
  Monkeypox virus B21R polypeptide 118
NRSSNQCFWSEPMILEDVSS

SEQ ID NO: 158 - protein, 20 aa, Artificial sequence
  Monkeypox virus B21R polypeptide 119
EPMILEDVSSCSSRTRKIYV

SEQ ID NO: 159 - protein, 20 aa, Artificial sequence
  Monkeypox virus B21R polypeptide 120
CSSRTRKIYVKLGIFNAEGF

SEQ ID NO: 160 - protein, 20 aa, Artificial sequence
  Monkeypox virus B21R polypeptide 121
KLGIFNAEGFNSFVLNCPTG

SEQ ID NO: 161 - protein, 20 aa, Artificial sequence
  Monkeypox virus B21R polypeptide 122
NSFVLNCPTGSTPTYIKHKN

SEQ ID NO: 162 - protein, 20 aa, Artificial sequence
  Monkeypox virus B21R polypeptide 123
STPTYIKHKNADSNNVIIEL

SEQ ID NO: 163 - protein, 20 aa, Artificial sequence
  Monkeypox virus B21R polypeptide 124
ADSNNVIIELPVGDYGTAKL

SEQ ID NO: 164 - protein, 20 aa, Artificial sequence
  Monkeypox virus B21R polypeptide 125
PVGDYGTAKLYSATKPSRIA

SEQ ID NO: 165 - protein, 20 aa, Artificial sequence
  Monkeypox virus B21R polypeptide 126
YSATKPSRIAVFCTHNYDKR

SEQ ID NO: 166 - protein, 20 aa, Artificial sequence
  Monkeypox virus B21R polypeptide 127
VFCTHNYDKRFKSDIIVLMF

SEQ ID NO: 167 - protein, 20 aa, Artificial sequence
  Monkeypox virus B21R polypeptide 128
FKSDIIVLMFNKNSGIPFWS

SEQ ID NO: 168 - protein, 20 aa, Artificial sequence
  Monkeypox virus B21R polypeptide 129
NKNSGIPFWSMYTGSVTSKN

SEQ ID NO: 169 - protein, 20 aa, Artificial sequence
  Monkeypox virus B21R polypeptide 130
MYTGSVTSKNRMFTTLARGM

SEQ ID NO: 170 - protein, 20 aa, Artificial sequence
  Monkeypox virus B21R polypeptide 131
RMFTTLARGMPFRSTYCDNR

SEQ ID NO: 171 - protein, 20 aa, Artificial sequence
  Monkeypox virus B21R polypeptide 133
RRSGCYYAGIPFHEDSVEAD

SEQ ID NO: 172 - protein, 20 aa, Artificial sequence
  Monkeypox virus B21R polypeptide 134
PFHEDSVEADIHYGPEIMLK

SEQ ID NO: 173 - protein, 20 aa, Artificial sequence
  Monkeypox virus B21R polypeptide 135
IHYGPEIMLKETYDINSIDP

SEQ ID NO: 174 - protein, 20 aa, Artificial sequence
  Monkeypox virus B21R polypeptide 136
ETYDINSIDPRVITKSKTHF

SEQ ID NO: 175 - protein, 20 aa, Artificial sequence
  Monkeypox virus B21R polypeptide 137
RVITKSKTHFPTPLSVKFMV

SEQ ID NO: 176 - protein, 20 aa, Artificial sequence
  Monkeypox virus B21R polypeptide 138
PTPLSVKFMVDNLGNGYDNP

SEQ ID NO: 177 - protein, 20 aa, Artificial sequence
  Monkeypox virus B21R polypeptide 139
DNLGNGYDNPNSFWEDAKTK

SEQ ID NO: 178 - protein, 20 aa, Artificial sequence
  Monkeypox virus B21R polypeptide 140
NSFWEDAKTKKRTYSAMTIK

SEQ ID NO: 179 - protein, 20 aa, Artificial sequence
  Monkeypox virus B21R polypeptide 142
VLPCTVRNKNIDFGYNYGDI

SEQ ID NO: 180 - protein, 20 aa, Artificial sequence
  Monkeypox virus B21R polypeptide 143
IDFGYNYGDIISNMVYLQST

SEQ ID NO: 181 - protein, 20 aa, Artificial sequence
  Monkeypox virus B21R polypeptide 144
ISNMVYLQSTSQDYGDGTKY

SEQ ID NO: 182 - protein, 20 aa, Artificial sequence
  Monkeypox virus B21R polypeptide 145
SQDYGDGTKYTFKSVTRSDH

SEQ ID NO: 183 - protein, 20 aa, Artificial sequence
  Monkeypox virus B21R polypeptide 146
TFKSVTRSDHECESSLDLTS

SEQ ID NO: 184 - protein, 20 aa, Artificial sequence
  Monkeypox virus B21R polypeptide 147
ECESSLDLTSKEVTVTCPAF

SEQ ID NO: 185 - protein, 20 aa, Artificial sequence
  Monkeypox virus B21R polypeptide 148
KEVTVTCPAFSIPRNISTYE

SEQ ID NO: 186 - protein, 20 aa, Artificial sequence
  Monkeypox virus B21R polypeptide 149
SIPRNISTYEGLCFSVTTSK

SEQ ID NO: 187 - protein, 20 aa, Artificial sequence
  Monkeypox virus B21R polypeptide 150
GLCFSVTTSKDHCATGIGWL

SEQ ID NO: 188 - protein, 20 aa, Artificial sequence
  Monkeypox virus B21R polypeptide 151
DHCATGIGWLKSSGYGKEDA

SEQ ID NO: 189 - protein, 20 aa, Artificial sequence
  Monkeypox virus B21R polypeptide 153
DKPRACFHHWNYYTLSLDYY

SEQ ID NO: 190 - protein, 20 aa, Artificial sequence
  Monkeypox virus B21R polypeptide 154
NYYTLSLDYYCSYEDIWRST

SEQ ID NO: 191 - protein, 20 aa, Artificial sequence
  Monkeypox virus B21R polypeptide 155
CSYEDIWRSTWPDYDPCKSY

SEQ ID NO: 192 - protein, 20 aa, Artificial sequence
  Monkeypox virus B21R polypeptide 156
WPDYDPCKSYIHIEYRDTWI

SEQ ID NO: 193 - protein, 20 aa, Artificial sequence
  Monkeypox virus B21R polypeptide 157
IHIEYRDTWIESNVLQQPPY

SEQ ID NO: 194 - protein, 20 aa, Artificial sequence
  Monkeypox virus B21R polypeptide 158
ESNVLQQPPYTFEFIHDNSN

SEQ ID NO: 195 - protein, 20 aa, Artificial sequence
  Monkeypox virus B21R polypeptide 160
EYVDKEISNKLNDLYNEYKK

SEQ ID NO: 196 - protein, 20 aa, Artificial sequence
  Monkeypox virus B21R polypeptide 161
LNDLYNEYKKIMEYSDGSLP

SEQ ID NO: 197 - protein, 20 aa, Artificial sequence
  Monkeypox virus B21R polypeptide 162
IMEYSDGSLPASINRLAKAL

SEQ ID NO: 198 - protein, 20 aa, Artificial sequence
  Monkeypox virus B21R polypeptide 163
ASINRLAKALTSEGREIASV

SEQ ID NO: 199 - protein, 20 aa, Artificial sequence
  Monkeypox virus B21R polypeptide 164
TSEGREIASVNIDGNLLDIA

SEQ ID NO: 200 - protein, 20 aa, Artificial sequence
  Monkeypox virus B21R polypeptide 165
NIDGNLLDIAYQADKEKMAD

SEQ ID NO: 201 - protein, 20 aa, Artificial sequence
  Monkeypox virus B21R polypeptide 167
IQTRINDIIRDLFIHTLSDK

SEQ ID NO: 202 - protein, 20 aa, Artificial sequence
  Monkeypox virus B21R polypeptide 169
DIKDIIESEEGKRCCIIDVK

SEQ ID NO: 203 - protein, 20 aa, Artificial sequence
  Monkeypox virus B21R polypeptide 171
NNRVKKYYSIDNYLCGTLDD

SEQ ID NO: 204 - protein, 20 aa, Artificial sequence
  Monkeypox virus B21R polypeptide 173
YIYTVVEYNKSYVLVNDTYM

SEQ ID NO: 205 - protein, 20 aa, Artificial sequence
  Monkeypox virus B21R polypeptide 174
SYVLVNDTYMSYDYLESSGV

SEQ ID NO: 206 - protein, 20 aa, Artificial sequence
  Monkeypox virus B21R polypeptide 175
SYDYLESSGVVVLSCYEMTI

SEQ ID NO: 207 - protein, 20 aa, Artificial sequence
  Monkeypox virus B21R polypeptide 176
VVLSCYEMTIISLDTKDAKD

SEQ ID NO: 208 - protein, 20 aa, Artificial sequence
  Monkeypox virus B21R polypeptide 177
ISLDTKDAKDAIEDVIVASA

SEQ ID NO: 209 - protein, 20 aa, Artificial sequence
  Monkeypox virus B21R polypeptide 179
VAEALNDMFKEFDKNVSAII

SEQ ID NO: 210 - protein, 20 aa, Artificial sequence
  Monkeypox virus B21R polypeptide 180
EFDKNVSAIIIKEEDNYLNS

SEQ ID NO: 211 - protein, 20 aa, Artificial sequence
  Monkeypox virus B21R polypeptide 181
IKEEDNYLNSSPDIYHIIYI

SEQ ID NO: 212 - protein, 20 aa, Artificial sequence
  Monkeypox virus B21R polypeptide 182
SPDIYHIIYIIGGTILLLLV

SEQ ID NO: 213 - protein, 20 aa, Artificial sequence
  Monkeypox virus B21R polypeptide 183
IGGTILLLLVIILILAIYIA

SEQ ID NO: 214 - protein, 20 aa, Artificial sequence
  Monkeypox virus B21R polypeptide 185
RNKYRTRKYEIMKYDNMSIK

SEQ ID NO: 215 - protein, 20 aa, Artificial sequence
  Monkeypox virus B21R polypeptide 186
IMKYDNMSIKSDHHDSLETV

SEQ ID NO: 216 - protein, 20 aa, Artificial sequence
  Monkeypox virus B21R polypeptide 187
KSDHHDSLETVSMEIIDNRY

SEQ ID NO: 217 - protein, 1783 aa, Variola virus
MNLQRLSLAIYLTVTCSWCYETCMRKTALFHDNQLGHAEDNQDSVASLPYKYLQVVNKRERSRLLATFNWTSIAEGVKNDFIRICDINGTYLYNYTIAVSMIIDSMEELPTITTYEPSTYNYTFDNSTVSTTEELKVTPSPTTYATVTTPLPTSSVPYDQRSNNNVSTISIQILSKILGVNETELTNYLITHKNATVDNNTLYGNIGFLEINNCYNISVSNASFRITLVNNTSEEIVIMLTGTSSSDTFISSTNITECLKTLINNTSNISDVSITQNMNVTSNCDKCSMNLMTSVIPAVKEFNNTLKKIGVKDDKNNTVYNYYNCKLTTNSTCDELINLDEVINNITLTNIISSSVSTTNSRKRRDLNGEFEFSTSEELDCLYKSYGVSDDVSHCFSSPRRRRSDDKQEYTEMKLLDHAKKDLRIDSVIPRGTTHFQVGASGASGGVVGDSSPFQNVKSRASLLAEKIMPRVPTTATEEQLYATINRQTKLPAGVKSTPFTEALVSTINQKLSSVKEVTYASLNLPGSSGYVHRPSDSVIYSTIRRTRLPSDTDSDFEDIQTVVKEYNERYGRRVSRTQSSSSDFEDIDEVVAEYRQKYGGASRGRTSSSSSSDFEDIDEVVAEYRQKYGNAMTKGRGSSKPDPLYSTVKKTPKSIASGVDIVSKQTDYSLLPGVNTGSSIVTPLTRRGATRRPKRPSTPPREDLPPLPLNPPYRQLSRGGDHSLQQVPQRDYSPPHRPPPPLPPKPVPAIPPSRDSQPNNKGFSKFVSPRRCRRSTSGVVCGMIQSRPNDDTYSLLQLPKIEPEYAEVGNGLPKNNVPVIGNKHSKKYTSSMSKISTKFDKSMAFGTAMLLTGQQAINQQDRSTALIKKDQMSKDEKIFEAVTMTLSTIGSTLTTAGMIAPPLMIAGIGISLISGIIDTAKDIYYLFSGQEKPVDPVIKFFNTYAGLVSDSSKMGVRKCLTPGEDTLIYIAYKNDSSFKQNTEAMALYFLDVINSEILYLNTSNLVLEYHLKVACPIGTLRSVDVDITAYTILYDTADNIKKYKFIRMATLLSKHPVIRLTCGLAATLVIKPYEVPISDMQLLKMATPGEPESTKSIPSDVCDRYPLKKFYLLAGGCPYDTSQTFIVHTTCSILLRTATWDQFRNRWVLQNPFRQEGTYKQLFTFSKYDFNDTIIDPNGVAGHASFCTNRSSNQCFWSEPMILEDVSSCSSRTRKIYVKLGIFNTEGFNSFVLNCPTGSTPTYIKDKNTDSNNVIIELPVGDYGTAKLYSVTKPSRIAVFCTHNYDKRFKSDIIVLIFNSISGIPFSSIYTGSVNGRNRLFTTLSKGMPYRSMYCDNRRPGCYYAGIPFNENSVESDLHYGPEIMLKETYDTNSIDPQVITKSKTHFPTPISVKFTVDNLGNGYNKPENFWKDAKSKKRTYSAMTIKILPCTVRNKNVDFGYNYGHIISNMVYAQSTSQDYGDGTNYTFKSVNRSDHECESILDLKAKEVTVMCPAFSIPRNISAYEGLCFSVTTSKDHCASNKEWLKSYGYGNTDATKQRVCFHHWNYVTTSLDYYCSYEDIWKSDWPDYDPCKSYIYIEYRDIWIESKVLQQPPYTFEFTHDDSNEYVNKEISNKLNDLYNEYKNIMEYSDGSLPASINRLAKSLTSEGREIASVNIDGNLLDIAYQADKEKMADIQNKINDITRDLFIHTLSNKDIKDIIESEEGKRCCIIDVKNNRVKKYYPIDNYLCGTLDDYIYTSVEYNKSYVLINDTYMSYDYLESSGVVVLSCYEMTIISLDT

(19) United States
(12) Patent Application Publication
(10) Pub. No.: US 2010/0316653 A1
(43) Pub. Date: Dec. 16, 2010

(54) COMPOSITIONS AND METHODS FOR DIAGNOSIS AND TREATMENT OF ORTHOPOXVIRUSES

(75) Inventors: Mark Slifka, Portland, OR (US); Paul Yoshihara, Portland, OR (US); Erika Hammarlund, Hillsboro, OR (US)

Correspondence Address: DAVIS WRIGHT TREMAINE, LLP/Seattle, 1201 Third Avenue, Suite 2200, SEATTLE, WA, 98101-3045, US

(73) Assignee: Oregon Health & Science University, Portland, OR (US)

(21) Appl. No.: 11/629,417

(22) PCT Filed: Jun. 13, 2005
(86) PCT No.: PCT/US05/20807

§ 371 Date: Dec. 24, 2008

Related U.S. Application Data
(60) Provisional application No. 60/579,048, filed on Jun. 12, 2004.

(51) Int. Cl. A61K 39/42; C12Q 1/70; C40B 30/04; C40B 40/10; C07K 16/08

(52) U.S. Cl. 424/159.1; 435/5; 506/9; 506/18; 530/387.1; 530/387.3; 530/388.3

(57) ABSTRACT

In particular aspects, the invention provides a novel approach for the systematic analysis and identification of biologically relevant epitopes (SABRE). SABRE-identified polypeptides have diagnostic (e.g., polypeptide arrays, etc.) and/or therapeutic (e.g., vaccines, etc.) utility, and utility for developing monoclonal antibodies having diagnostic and/or therapeutic utility (e.g. for detecting and/or preventing orthopoxvirus infection). Preferred aspects provide high-throughput assays for detecting specific orthopoxvirus infection, for detecting orthopoxvirus-specific immune response, or for dual (parallel) determination of both orthopoxvirus immune response and orthopoxvirus infection. Additional preferred and surprising aspects provide novel high-throughput methods for detecting ‘protective immunity’ against orthopoxviruses (e.g., for detecting protective immunity against smallpox virus and monkeypox virus), based on anti-vaccinia virus serum antibody levels. The inventive diagnostic assays are rapid, high-throughput and suitable for ‘point-of-care’ implementations.

### CROSS-REFERENCE TO RELATED APPLICATIONS

The application claims the benefit of priority to U.S. Provisional Patent Application Ser. No. 60/579,048, filed 12 Jun. 2004, which is incorporated by reference herein in its entirety.

### FIELD OF THE INVENTION

The invention generally relates generally to orthopoxviruses (e.g., smallpox, vaccinia and monkeypox), and more particularly to diagnostic and therapeutic methods comprising use of orthopoxvirus proteins, polypeptides and anti-orthopoxvirus antibodies. Additionally, the invention relates to novel methods for systematic analysis of biologically relevant epitopes (SABRE™).

### BACKGROUND

Orthopoxviruses. Orthopox viruses, including smallpox, monkeypox and vaccinia viruses, cause a number of contagious infections, and can be fatal. Smallpox, for example, is a highly contagious, often fatal disease caused by variola virus. About 30% of those infected with the smallpox virus die. Smallpox outbreaks had occurred periodically for thousands of years. Fortunately, naturally occurring smallpox virus was eliminated worldwide in 1978 through the outstanding efforts of the WHO Global Eradication Program. Nonetheless, there is an ongoing concern that terrorists, or rogue nations or states might be able to obtain, or potentially create, a deposit of smallpox and develop a biological weapon of mass destruction. Such concerns are legitimate for several reasons.

Scientists have retained stocks of the variola virus for research purposes in two secure laboratories, one at the CDC in Atlanta, Ga., and the other in Moscow, Russia. The CDC has classified smallpox as the highest priority (Category A) bioterrorism threat to the U.S. public health system and national security due to the fact that variola virus can be easily disseminated and transmitted from person to person, has the potential to cause widespread illness and death, and requires special actions for public health preparedness. Additionally, there is currently no specific treatment for smallpox disease, and the only prevention is vaccination.

Moreover, and significantly, the last mass vaccination was in the mid 1970's, and through this highly successful vaccination program, >90% of Americans over the age of 35 (˜140 million people; 2000 U.S. Census Bureau) have already been vaccinated against smallpox. Nonetheless, current views on smallpox immunity suggest that residual immunity against smallpox and vaccinia is questionable, being low or non-existent in today's population, because vaccination using vaccinia virus for immunization against smallpox occurred many years ago (roughly 25 to 75 years ago.

Prior art detection of orthopoxviruses. The ability to rapidly respond to a potential outbreak initially depends upon the availability of assays suitable for rapid and specific detection of the condition or agent before substantial communication thereof. Preferably, such assays should be virus specific, and should allow for detection of exposure to orthopoxvirus before the active stages of the disease; that is, prior to formation of skin lesions.

PCR-based assays. While very sensitive PCR-based detection methods for orthopoxviruses are available, these assays have significant disadvantages. One disadvantage is that PCR assays require specialized equipment and uncontaminated reagents, and, in the orthopoxvirus context, are typically performed in a limited number of specialized centers. Such PCR-based assays are thus not readily available as facile ‘first response’-type ‘field’ assays systems. Furthermore, PCR techniques detect specific polynucleotides that are present during viral replication, and are thus only effective in active stages of the disease; that is, when skin lesions are showing. This is a relatively narrow time window, and thus false-negative results may be obtained. For example, during a recent monkeypox outbreak in Wisconsin, there was at least one case where a person, who owned a prairie dog that had died of a monkeypox virus infection, but who tested negative for the monkeypox virus by the PCR-based assay. This individual had all of the standard clinical symptoms of a monkeypox infection including pox lesions, but failed to go to the hospital during the early stages of the disease. While an ELISA test showed that this person was infected by the monkeypox virus, the PCR-based assay failed to detect the virus.

Plaque-reduction assays. In practice, the vaccinia plaque-reduction test can be used to determine the serum dilution at which 50% of the infectious virus (e.g., vaccinia) is neutralized (NT₅₀). The disadvantage of this assay, however, are that it is time consuming, cumbersome and cannot be used as a rapid, high-throughput platform. Historically, the vaccinia plaque-reduction test was employed for determining anti-smallpox immunity by indirectly measuring the levels of vaccinia-specific neutralizing antibodies in the serum.

ELISA. Currently, rapid and relatively facile ELISA-based assays are available, in some cases, to quantify virus-specific Ig levels. However, orthopoxvirus-specific ELISA platforms do not exist for all orthopoxviruses (e.g., monkeypox). Additionally, as widely recognized in the art, ELISA assays of serum antibodies are uniformly regarded as not having utility for determination of protective immunity.

In summary, while very sensitive PCR-based assays exist, they are applicable over a relatively narrow window of infection, and are not suited to ‘first response’-type ‘field’ conditions. Moreover, while plaque-reduction tests are available, they are cumbersome and not suited for rapid, high-throughput conditions. Furthermore, while ELISA-based assays are available, they are regarded as having no utility for determination of protective immunity, and are not specific, in some cases to a particular virus (e.g., as in the case of monkeypox virus).

Therefore, there is a pronounced need in the art for reliable and efficient methods for the detection of viral infection, including detecting a viral infection during all stages, rather than detecting the virus only when it is in its replicative stage.

There is a pronounced need in the art for reliable and efficient methods for dual or parallel detection of monkeypox virus (MPV) infection, and MPV-specific immune response.

There is a pronounced need in the art for reliable and efficient detection of protective immunity against orthopoxviruses, including smallpox.

There is a pronounced need in the art for novel anti-MPV antibodies, and antibody compositions comprising anti-MPV antibodies, and methods of treatment and prevention using anti-MPV antibodies and/or compositions comprising anti-MPV antibodies.

### SUMMARY OF THE INVENTION

In particular aspects, the invention provides a novel approach for systematic analysis of biologically relevant epitopes (SABRE) having substantial utility for rapidly and effectively mapping biologically relevant peptide epitopes suitable for novel diagnostic and/or therapeutic applications.

Particular embodiments provide for using the SABRE-identified polypeptides to develop monoclonal antibodies, and compositions comprising such antibodies, having substantial utility as novel diagnostic reagents for detecting the respective pathogen (e.g. for detecting orthopoxvirus infection). The diagnostic assays are rapid, high-throughput and suitable for ‘point-of-care’ implementations.

Additional embodiments provide for using SABRE-identified polypeptides to develop monoclonal antibodies, and compositions comprising such antibodies, for novel therapeutic use for treatment or prevention of orthopoxvirus (e.g., smallpox, monkeypox and vaccinia) infections, comprising using the inventive antibodies and antibody compositions to treat an infection, to alleviate symptoms of the infection, and/or to help prevent pathogen infection.

Yet additional embodiments provide vaccines, based on the use of one or more SABRE-identified antigens in vaccine compositions.

Further embodiments provide for using the SABRE-identified polypeptides to develop novel high-throughput assays for the detection of orthopoxvirus-specific immune response, based on measurement of orthopoxvirus-specific serum antibody levels. The diagnostic assays are rapid, high-throughput and suitable for ‘point-of-care’ implementations.

Yet additional embodiments provide for using the SABRE-identified polypeptides and respective antibodies in high-throughput methods for dual (parallel) determination of orthopoxvirus immune response and orthopoxvirus infection. The diagnostic assays are rapid, high-throughput and suitable for ‘point-of-care’ implementations.

Further embodiments provide for using SABRE-identified polypeptides and respective antibodies in high-throughput methods for determination of orthopoxvirus-specific (e.g., smallpox-specific, monkeypox-specific, smallpox/monkeypox-specific) immune response and orthopoxvirus infection.

Yet further embodiments provide an array of different orthopoxvirus (e.g., monkeypox virus) peptide epitopes coupled to a solid phase.

In yet additional aspects, the present invention represents a surprising departure from the long-standing art-recognized dogma that particular immunological (e.g., ELISA) assays have no utility for determination of protective immunity against orthopoxviruses, and particular embodiments provide rapid and reliable high-throughput methods for detecting protective immunity against orthopoxviruses (e.g., for determination of protective immunity against smallpox virus, based on anti-vaccinia virus serum antibody levels. The diagnostic assays are rapid, high-throughput and suitable for ‘point-of-care’ implementations.

### BRIEF DESCRIPTION OF THE DRAWINGS

FIGS. 1A and 1B show the levels of Virus-specific CD4⁺ T cell memory following smallpox vaccination.

FIGS. 2A and B show the levels of virus-specific CD8⁺ T cell memory following smallpox vaccination.

FIGS. 3A, 3B and 3C show the relationship between vaccinia-specific CD4⁺ and CD8⁺ T cell memory over time. Comparisons were made between the number of antiviral CD4⁺ and CD8⁺ T cells from the same individual. FIGS. 3A, 3B, and 3C show 1 month to 7 years post-vaccination (p.v.), 14 to 40 years p.v., and 41 to 75 years p.v., respectively.

FIGS. 4A, 4B and 4C show long-lived antiviral antibody responses induced by smallpox vaccination. FIGS. 4A, 4B, and 4C show the quantitation of vaccinia-specific antibody responses by ELISA (4A), the levels of vaccinia-specific antibody titers (1 to 75 years post-vaccination) compared to the total number of vaccinations received (4B), and the correlation between virus-specific antibody titers and neutralizing antibodies (4C), respectively.

FIGS. 4D, 4E and 4F show the relationship between virus-specific CD4⁺ (closed symbols) or CD8⁺ (open symbols) T cells (per million CD4⁺ or CD8⁺ T cells, respectively) with virus-specific antibody titers as determined at 1 month to 7 years post-vaccination (p.v.) (4D), 14 years to 40 years p.v. (4E), and 41 years to 75 years p.v. (4F), respectively.

FIGS. 5A-5D show antiviral antibody responses following orthopoxvirus infection (see EXAMPLE V herein below).

FIG. 6 shows diagnosis of recent monkeypox infection by quantitation of orthopoxvirus-specific T cells. The frequency of virus-specific T cells capable of producing both IFN□ and TNF□ after direct ex vivo stimulation with vaccinia virus was determined by intracellular cytokine staining (ICCS).

FIG. 7 shows analysis of monkeypox-specific peptide ELISA assays for diagnosing monkeypox infection. Serum or plasma samples (1:50 dilution) obtained at 2 months to 1 year post-infection/exposure were incubated on ELISA plates coated with an individual peptide in each well. Samples were scored positive for a particular peptide if they scored ≧2-fold over background on at least 2 to 3 different ELISA plates.

FIG. 8 shows the relationship between reported and unreported (i.e. asymptomatic) monkeypox infections. This figure was modified from a similar flow-chart diagram published by Reed et al. (11) and shows the relationship between different monkeypox survivors in the context of the WI monkeypox outbreak. Patients 4 and 5 are subjects who purchased 39 prairie dogs from an Illinois distributor and sold 2 prairie dogs to the family in the Northwestern WI household, the site of the first recorded case of human monkeypox in the United States.

FIG. 9 shows a comparison of the number of monkeypox lesions reported by unvaccinated and vaccinated monkeypox patients. Subjects were asked to fill out a medical history questionnaire describing their history of monkeypox infection including the number of monkeypox lesions or “pocks” that developed during the course of this acute viral infection.

### DETAILED DESCRIPTION OF THE INVENTION

In particular aspects, the invention provides a novel approach, herein referred to as SABRE, for systematic analysis of biologically relevant epitopes of pathogen proteins. SABRE provides for rapid and effective mapping and identification of biologically relevant peptide epitopes of pathogen proteins that are suitable for novel diagnostic and/or therapeutic applications. Preferred pathogen proteins are those of the orthopoxviruses (e.g., smallpox, vaccinia and monkeypox). The diagnostic assays are rapid, high-throughput and suitable for ‘point-of-care’ implementations.

SABRE-identified polypeptides have utility for developing respective antibodies (e.g., monoclonal antibodies), and compositions comprising such antibodies, having utility as novel diagnostic reagents for detecting the respective pathogen (e.g. for detecting orthopoxvirus infection). The diagnostic assays are rapid, high-throughput and suitable for ‘point-of-care’ implementations.

SABRE-identified polypeptides have utility for developing antibodies (e.g., monoclonal antibodies), and compositions comprising such antibodies, having therapeutic utility for treatment or prevention of orthopoxvirus (e.g., smallpox, monkeypox and vaccinia) infections. The inventive antibodies and antibody compositions have utility for treating an infection, for alleviating symptoms of an infection, and/or to prevent pathogen infection.

The SABRE-identified polypeptides provide vaccines, based on the use of one or more SABRE-identified antigens in vaccine compositions.

The SABRE-identified polypeptides were used herein to develop novel high-throughput assays for the detection of orthopoxvirus-specific immune response, based on measurement of orthopoxvirus-specific serum antibody levels. The diagnostic assays are rapid, high-throughput and suitable for ‘point-of-care’ implementations.

Additionally, according to the present invention, the SABRE-identified polypeptides and respective antibodies have utility for use in a high-throughput method for dual (parallel) determination of orthopoxvirus immune response and orthopoxvirus infection. The diagnostic assays are rapid, high-throughput and suitable for ‘point-of-care’ implementations.

Yet further aspects, the present invention provides an array of different orthopoxvirus (e.g., monkeypox virus) peptide epitopes (e.g., coupled to a solid phase).

In yet additional aspects, the present invention represents a surprising departure from the long-standing art-recognized dogma that particular immunological (e.g., ELISA) assays have no utility for determination of protective immunity against orthopoxviruses, and particular embodiments provide rapid and reliable high-throughput methods for detecting protective immunity against orthopoxviruses (e.g., for determination of protective immunity against smallpox virus, based on anti-vaccinia virus serum antibody levels. The diagnostic assays are rapid, high-throughput and suitable for ‘point-of-care’ implementations.

### Definitions

to “Protective immunity” refers to the art-recognized protective immunity by a host, the immunity having been induced within the host by one or more prior vaccinations, or by one or more prior pathogen infections.

“Passive immunity” or “Immediate immunity” refers to the immunity conferred within a host, by passive antibody administration, wherein, passive antibody can theoretically confer protection regardless of the immune status of the host. Passive antibody administration can be used for post-exposure prophylaxis.

The term “SABRE” is an acronym for a novel method as disclosed herein for systematic analysis of biologically relevant epitopes.

The term “epitope” refers herein, as is known in the art, to an antigenic determinant of a protein of polypeptide. An epitope could comprise 3 amino acids in a spacial conformation which is unique to the epitope. Generally an epitope consists of at least 5 such amino acids. An epitope of a polypeptide or protein antigen can be formed by contiguous or noncontinguous amino acid sequences of the antigen. A single viral protein, for example, may contain many epitopes. Additionally, a polypeptide fragment of a viral protein may contain multiple epitopes. The present invention encompasses epitopes and/or polypeptides recognized by antibodies of the present invention, along with conservative substitutions thereof, which are still recognized by the antibodies. Further truncation of these epitopes may be possible.

The term “Poxviridae” refers to viruses in the family Poxviridae, including poxviruses in the genera orthopoxvirus, parapoxvirus, avipoxvirus, capripoxvirus, leporipoxvirus, suipoxvirus, molluscipoxvirus and Yatapoxvirus which members include variola major and minor virus, monkeypox virus, camelpox virus, raccoonpox virus, ectromelia virus, sealpox virus, contagious ecthyma virus, canarypox virus, juncopox virus, pigeonpox virus, turkeypox virus, penguinpox virus, sheepox virus, goatpox, swinepox virus, buffalopox virus, cowpox virus, rabbit fibroma virus, myxoma virus, and molluscum contagiosum (genus Molluscipoxvirus) which is 59% identical and 77% similar to vaccinia (Altschul, S. F. et al. 1997, Nucl. Acids Res. 25, 3389-3402, fowlpox (genus Avipoxvirus), Yata-tumor like virus (Yatapoxvirus), among others (Fenner, Frank, Poxviruses, In “Virology” B. N. Fields et al., eds. Raves Press, Ltd. New York, 1990, pp. 2113-2133).

“Orthopoxviruses” refers, within the Poxviridae family, to a genus of closely related viruses that includes, but is not limited to, variola (smallpox), vaccinia, cowpox and monkeypox (all of which are known to infect humans), and also includes, but is not limited to camelpox, raccoonpox, skunkpox, volepox, ectromelia, and gerbilpox viruses.

“ELISA” refers to enzyme-linked immuno sorbent assays, as widely recognized in the art, and as described herein.

“Immunologic assay,” as used herein refers to an art-recognized immunologic assay suitable to detect the formation of antigen:antibody complexes, including, but not limited to antibody capture assays; antigen capture assays, and two-antibody sandwich assays, ELISA, immunodiffusion, immunoelectrophoresis, immunochemical methods, binder-ligand assays, immunohistochemical and immuncytochemical techniques, Western analysis, agglutination and complement assays (see e.g., Basic and Clinical Immunology, 217-262, Sites and Terr, eds., Appleton & Lange, Norwalk, Conn., 1991 which is incorporated herein by reference). Preferred embodiments (e.g., ELISA) of such assays are described herein below. According to the present invention, one or more of such immunoassays can be used to detect and/or quantitate antigens (e.g., Harlow & Lane, Antibodies: A Laboratory Manual; Cold Spring Harbor Laboratory, New York 555-612, 1988, incorporated by reference herein).

The term “treatment” refers to both therapeutic treatment and prophylactic or preventative measures, wherein the object is to prevent or slow (lessen) pathogen (e.g., viral) infection or associated conditions. Those in need of treatment include those already experiencing an infection, those prone to infection, and also those in which the potential infection is to be prevented.

“Antibodies,” as used herein, refers to the art-recognized definition, and are described in more detail herein below.

“Neutralizing antibodies,” as used herein, refers to the art-recognized definition.

“Cognate antigen,” as used herein, refers to an antigen that is specifically bound by a cognate antibody, and “cognate antibody” refers to the antibody that specifically binds a cognate antigen.

“Parallel” or “dual” detection, as used herein refers to, detection, within a single sample, of both MPV infection and MPV-specific immune response. Preferably, detection of infection is contemporaneous with detection of a respective immune response to enable combined diagnostic use, but need not be simultaneous, and a plurality of immunologic assays and reagents. Preferably, parallel detection comprises use of at least one antigen, for detection of immune response, that is a cognate antigen of an antibody reagent used for detection of viral infection in the same sample.

“Orthpoxvirus proteins and polypeptides” as used herein encompasses both full-length orthopoxvirus proteins, as well as portions of such proteins, and includes ‘peptides’ and ‘oligopeptides,’ and additionally includes functional (e.g., epitope-bearing, or antibody-binding) variants (including conservative amino acid sequence variants as described herein), fragments, muteins, derivatives and fusion proteins thereof.

“Vaccine,” as used herein and in the art, refers to any type of biological agent in an administratable forth capable of stimulating an immune response in an animal inoculated with the vaccine. For purposes of preferred embodiments of this invention, an inventive vaccine may comprise as the viral agent, one or more immunogenic (antigenic) components of the virus (e.g., see TABLE 2 herein below for preferred antigens), and including polypeptide-based vaccines.

### SABRE Technology (Systematic Analysis of Biologically Relevant Epitopes)

Preferred aspects of the present invention provide novel methods for systematic analysis of biologically relevant epitopes (SABRE), which enable rapidly and effective mapping/identification of biologically relevant (e.g., immunodominant) peptide epitopes suitable for diagnostic and/or therapeutic applications.

Prior art methods for identification of biologically relevant peptide antigen/epitopes are “shotgun” approaches whereby a panel of uncharacterized antibodies, elicited by a particular antigen, are subsequently screened and tested to characterize the antibodies (e.g., class, affinity, specificity, etc) to facilitate elucidation of the biological' relevancy of the particular antigen/epitope. For example, a panel of antibodies generated against a particular viral antigen, might be screened and tested for the ability of the antibodies to neutralize virus and/or protect mice from viral challenge. Thus, such prior art approaches have great utility, once a biologically relevant antigen/epitope has been identified, but they do not provide an efficient method for initial selection of a biologically relevant antigen/epitope from among a large number of potentially relevant antigens and epitopes.

For example, U.S. Pat. No. 6,620,412 to Hooper et al teaches a method for identification of potential targets for poxvirus therapeutics, comprising: initially generating a panel of 400 VACV-specific monoclonal antibodies (MAbs) in mice; and then characterizing the monoclonal antibodies by testing for their ability to neutralize virus and/or their ability to protect mice from challenge. Hooper et al used two challenge models, one that involves dissemination of the virus (in suckling mice), and another that involves a massive challenge dose (by intraperitoneal injection). Likewise, other prior art approaches are based on the same paradigm; namely, methods characterized by generation of antigen specific panel of antibodies, and subsequent characterization or properties and biological relevance.

The instant inventive systematic analysis of biologically relevant epitopes (SABRE) method provides a novel approach for rapidly and effectively mapping biologically relevant (e.g., immunodominant) peptide epitopes suitable for diagnostic and/or therapeutic applications.

In preferred aspects, the method comprises: obtaining acute and/or convalescent serum from patients or naturally/experimentally infected animals who have recovered from a specific infectious disease or who are in the process of recovering from a specific infectious disease; obtaining specific polypeptides representing sub-regions of one or more proteins relevant to the infectious agent (e.g., a set of polypeptides, based on genomic sequences and hydrophobicity plots) and using these polypeptides (e.g., to create an array of polypeptides; to coat ELISA plates) for screening against positive and negative control sera; and identifying polypeptides/epitopes with high reactivity to positive control sera (e.g., immunodominant epitopes) and low reactivity to negative control sera, thereby identifying biologically relevant epitopes.

Preferred proteins and polypeptides. Preferred proteins and polypeptides of the present invention are those of pathogenic viruses, such as orthopoxvirus proteins (e.g., smallpox, vaccinia and monkeypox). In particular embodiments, they are of a strain of monkeypox virus. In other embodiments, they are a monkeypox virus (MPV) protein or polypeptide antigen selected from the group consisting of D2L, N2R, N3R, B18R, B21R and epitope-bearing fragments of D2L, N2R, N3R, B18R and B21R. In particular embodiments, the proteins and polypeptides are selected from the group consisting of those listed in TABLE 2, herein below (SEQ ID NOS:1-29), and in TABLES 4, 5 and 6 (e.g., SEQ ID NOS:30-44).

In particular embodiments, the monkeypox protein or polypeptide comprises at least one epitope of a sequence selected from the group consisting of SEQ ID NOS:1 (MPV D2L), 6 (MPV N2RR), 10 (N3R), 16 (B18R) and 20 (B21R), and epitope-bearing fragments of SEQ ID NOS:1 (MPV D2L), 6 (MPV N2R), 10 (MPV N3R), 16 (MPV B18R) and 20 (MPV B21R).

In particular embodiments, the monkeypox protein or polypeptide comprises at least one epitope of a sequence selected from the group consisting of SEQ ID NOS:2-5 (MPV D2L), 7-9 (MPV N2R), 11-15 (MPV N3R), 17-19 (MPV B18R) and 21-29, 30-44 (MPV B21R), and epitope bearing fragments of SEQ ID NOS:2-5, 7-9, 11-15, 17-19, 21-29 and 30-44.

In particular embodiments, the monkeypox protein or polypeptide comprises at least one epitope of a sequence selected from the group consisting of SEQ ID NOS:10 (MPV N3R) and 20 (MPV B21R), and epitope-bearing fragments of SEQ ID NOS:10 and 20.

In particular embodiments, the monkeypox protein or polypeptide comprises at least one epitope of a sequence selected from the group consisting of SEQ ID NOS:11-15 (MPV N3R) and 21-29 (MPV B21R), and epitope bearing fragments of SEQ ID NOS: 11-15, 21-29 and 30-44.

In particular embodiments, the epitope comprises a sequence selected from the group consisting of SEQ ID NOS:15 (MPV N3R₁₅₇₋₁₇₆) and 27 (MPV B21R₇₂₉₋₇₄₈), and epitope-bearing fragments of SEQ ID NOS:15 and 27.

In particular embodiments, the epitope comprises a sequence selected from the group consisting of SEQ ID NO:31 and epitope-bearing fragments of SEQ ID NO:31.

Vaccines. In particular embodiments, the SABRE-identified polypeptides provide vaccines, based on the use of one or more SABRE-identified antigens in vaccine compositions. Such peptide-based vaccines are well known in the art, and may contain additional antigenic and adjuvant elements. Peptide-based vaccine are advantageous over traditional vaccines for several reasons: they are substantially safer; they have a relatively long shelf-life; they have the ability to target the immune response towards specific epitopes that are not suppressive nor hazardous for the host; and they offer the possibility of preparing multi-component and multi-pathogen vaccines.

The efficacy of inventive peptide-based vaccines are enhanced by adequate presentation of the epitopes to the immune system. Therefore, in preferred aspects, the orthopoxvirus (e.g., monkeypox) antigens/epitopes are couple to, or are expressed (e.g, hydrid-gene expression) as part of, a carrier that may also offer an adjuvant function. Additional adjuvants may or may not be included in the immunization.

In particular aspects, immunizations are performed with one or more monkeypox virus (MPV) protein or polypeptide antigens selected from the group consisting of D2L, N2R, N3R, B18R, B21R and epitope-bearing fragments of D2L, N2R, N3R, B18R and B21R. In particular embodiments, the MPV protein or polypeptide is selected from the group consisting of SEQ ID NOS:10, 20 and epitope-bearing fragments of SEQ ID NOS:10 and 20. In particular embodiments, the MPV protein or polypeptide is selected from the group consisting of SEQ ID NOS:11-15, 21-29 and epitope bearing fragments of SEQ ID NOS:11-15, 21-29 and 30-44. In particular embodiments, the MPV protein or polypeptide is selected from the group consisting of SEQ ID NOS:15 (MPV N3R₁₅₇₋₁₇₆), 27 (MPV B21R₇₂₉₋₇₄₈), and epitope-bearing fragments of SEQ ID NOS:15 and 27. In particular embodiments, the MPV protein or polypeptide is selected from the group consisting of SEQ ID NO:31 and epitope bearing fragments of SEQ ID NO:31.

Antibodies. In particular embodiments, SABRE-identified polypeptides have utility for developing respective antibodies (e.g., monoclonal antibodies), and compositions comprising such antibodies.

Such antibodies and compositions have utility as novel diagnostic reagents for directly detecting the respective pathogen (e.g. for detecting orthopoxvirus infection, such as monkeypox infection). The diagnostic assays are rapid, high-throughput and suitable for ‘point-of-care’ implementations.

Diagnostic assays. Particular aspects of the present invention thus provide A high-throughput method for detecting monkeypox virus (MPV) infection, comprising: obtaining a test serum sample from a test subject; and detecting MPV in the sample using an immunologic assay based, at least in part, on use of at least one antibody reagent, or epitope-binding portion thereof, specific for an MPV protein or polypeptide antigen selected from the group consisting of D2L, N2R, N3R, B18R, B21R and epitope-bearing fragments of D2L, N2R, N3R, B18R and B21R.

In particular embodiments, the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20. In particular embodiments, the MPV polypeptide antigen is selected from the group consisting of SEQ ID NOS:2-5, 7-9, 11-15, 17-19, 21-29, 30-44 and epitope bearing fragments of SEQ ID NOS:2-5, 7-9, 11-15, 17-19, 21-29 and 30-44. Preferably, the MPV protein or polypeptide anti_{g}en is selected from the group consisting of SEQ ID NOS:10, 20 and epitope-bearing fragments of SEQ ID NOS:10 and 20. In particular embodiments, the MPV polypeptide antigen is selected from the group consisting of SEQ ID NOS:11-15, 21-29, 30-44 and epitope bearing fragments of SEQ ID NOS:11-15, 21-29 and 30-44. In particular embodiments, the MPV polypeptide antigen is selected from the group consisting of SEQ ID NOS:15 (MPV N3R₁₅₇₋₁₇₆), 27 (MPV B21R₇₂₉₋₇₄₈), and epitope-bearing fragments of SEQ ID NOS:15 and 27. In particular embodiments, the MPV polypeptide antigen is selected from the group consisting of SEQ ID NO:31 and epitope-bearing fragments of SEQ ID NO:31. In particular embodiments, the immunologic assay is selected from the group consisting of ELISA, immunoprecipitation, immunocytochemistry, immunoelectrophoresis, immunochemical methods, Western analysis, antigen-capture assays, two-antibody sandwich assays, binder-ligand assays, agglutination assays, complement assays, and combinations thereof. In particular embodiments, the antibody is selected from the group consisting of a single-chain antibody, a monoclonal antibody, a chimeric antibody, a humanized antibody, and a Fab fragment. In particular embodiments, a plurality of antibodies, or eptitope-binding portions thereof, are used, in each case specific for an MPV protein or polypeptide antigen selected from the group consisting of D2L, N2R, N3R, B18R, B21R and epitope-bearing fragments of D2L, N2R, N3R, B18R and B21R.

Therapeutic agents. Additionally, because of the nature of the relevant specific binding interactions, antibodies and antibody-containing compositions of the present invention have therapeutic utility for treatment or prevention of orthopoxvirus (e.g., smallpox, monkeypox and vaccinia) infections. The inventive antibodies and antibody compositions have utility for treating an infection, for alleviating symptoms of an infection, and/or to prevent pathogen infection. Preferably, the antibodies and antibody compositions are directed against monkeypox virus, or monkeypox virus proteins or polypeptides, and can be used to treat or prevent monkeypox virus infection by administration to subjects in need thereof.

Specifically, particular embodiments of the present invention provide an antibody directed against a monkeypox virus (MPV) protein or polypeptide antigen selected from the group consisting of D2L, N2R, N3R, B18R, B21R and epitope-bearing fragments of D2L, N2R, N3R, B18R and B21R.

In particular embodiments, the antibody is a monoclonal antibody, or antigen-binding portion thereof. In particular embodiments, the monoclonal antibody, or antigen-binding portion thereof, is a single-chain antibody, chimeric antibody, humanized antibody or Fab fragment. Preferably, the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20. In particular embodiments, the monkeypox virus (MPV) polypeptide antigen is selected from the group consisting of SEQ ID NOS:2-5, 7-9, 11-15, 17-19, 21-29, 30-44 and epitope bearing fragments of SEQ ID NOS:2-5, 7-9, 11-15, 17-19, 21-29 and 30-44. In particular embodiments, the monkeypox virus (MPV) protein or polypeptide antigen is selected from the group consisting of SEQ ID NOS:10, 20 and epitope-bearing fragments of SEQ ID NOS:10 and 20. In particular embodiments, the monkeypox virus (MPV) polypeptide antigen is selected from the group consisting of SEQ ID NOS:11-15, 21-29, 30-44 and epitope bearing fragments of SEQ ID NOS:11-15, 21-29 and 30-44. In particular embodiments, the monkeypox virus (MPV) polypeptide antigen is selected from the group consisting of SEQ ID NOS:15 (MPV N3R₁₅₇₋₁₇₆), 27 (MPV B2R₇₂₉₋₇₄₈), and epitope-bearing fragments of SEQ ID NOS:15 and 27. In particular embodiments, the monkeypox virus (MPV) polypeptide antigen is selected from the group consisting of SEQ ID NO:31 and epitope-bearing fragments of SEQ ID NO:31.

Additional aspects provide a composition, comprising at least one of the above-described antibodies. Preferably, the composition comprises a N3R-specific monoclonal antibody, and a B21R-specific monoclonal antibody. Preferably, at least one of the antibodies forms specific immunocomplexes with monkeypox whole virions, or proteins or polypeptides associated with monkeypox virions.

Yet further aspects provide a pharmaceutical composition, comprising at least one of the above-described antibodies of, along with a pharmaceutically acceptable diluent, carrier or excipient. Preferably, the composition is administered to a subject, whereby the composition prevents or inhibits monkeypox virus infection. In particular embodiments, the composition is administered to a subject, whereby the composition ameliorates symptoms of monkeypox virus infection. In particular embodiments, at least one of the antibodies of the composition forms specific immunocomplexes with monkeypox whole virions, or proteins or polypeptides associated with monkeypox virions.

Yet further aspect provide a method of treating, or of preventing monkeypox virus infection, comprising administering to a subject in need thereof, a therapeutically effective amount of at least one of the above-described antibodies, or of a pharmaceutical composition comprising at least one of the antibodies. In particular embodiments, the immunoglobulin sequences are, or substantially are, human immunoglobulin sequences.

Detection of orthopoxvirus-specific immune response. In additional aspects, the present invention provides novel high-throughput assays for the detection of orthopoxvirus-specific immune response, based on measurement of orthopoxvirus-specific serum antibody levels. The diagnostic assays are rapid, high-throughput and suitable for ‘point-of-care’ implementations. Preferably, the orthopoxviruses include, but are not limited to smallpox, monkeypox and vaccinia viruses. EXAMPLE IV, herein below, describes the use of SABRE-identified polypeptides for detection of monkeypox virus-specific immune response (see also EXAMPLES V and VI).

Particular aspects provide a high-throughput method for detecting a monkeypox virus (MPV)-specific immune response, comprising: obtaining a test serum sample from a test subject; and detecting MPV-specific antibodies in the sample using an immunologic assay, based, at least in part, on use of at least one MPV protein or polypeptide selected from the group consisting of D2L, N2R, N3R, B18R, B21R, epitope-bearing fragments of D2L, N2R, N3R, B18R and B21R, and combinations thereof.

In particular embodiments, the monkeypox virus (MPV) protein or polypeptide is selected from the group consisting of SEQ ID NOS:1, 6, 10, 16, 20, and epitope-bearing fragments of SEQ ID NOS:1, 6, 10, 16 and 20. Preferably, the MPV polypeptide is selected from the group consisting of SEQ ID NOS:2-5, 7-9, 11-15, 17-19, 21-29, 30-44 and epitope bearing fragments of SEQ ID NOS:2-5, 7-9, 11-15, 17-19, 21-29 and 30-44. Preferably, the MPV protein or polypeptide is selected from the group consisting of SEQ ID NOS:10, 20 and epitope-bearing fragments of SEQ ID NOS:10 and 20. Preferably, the MPV polypeptide is selected from the group consisting of SEQ ID NOS:11-15, 21-29, 30-44 and epitope bearing fragments of SEQ ID NOS:11-15, 21-29 and 30-44. In particular embodiments, the MPV polypeptide is selected from the group consisting of SEQ ID NOS:15 (MPV N3R₁₅₇₋₁₇₆), 27 (MPV B21R₇₂₉₋₇₄₈), and epitope-bearing fragments of SEQ ID NOS:15 and 27. In particular embodiments, the MPV polypeptide is selected from the group consisting of SEQ ID NO:31 and epitope-bearing fragments of SEQ ID NO:31. In particular embodiments, the immunologic assay is selected from the, group consisting of ELISA, immunoprecipitation, immunocytochemistry, Western analysis, antigen capture assays, two-antibody sandwich assays and combinations thereof. In particular embodiments, a plurality of MPV proteins or polypeptides are used, in each case selected from the group consisting of D2L, N2R, N3R, B18R, B21R and epitope-bearing fragments of D2L, N2R, N3R, B18R and B21R.

In particular embodiments, detecting monkeypox virus (MPV)-specific antibodies in the sample further comprises determining an amount of MPV-specific antibodies in the sample, and the method further comprises determining, based at least in part on the amount of MPV-specific antibodies, a corresponding amount of MPV-neutralizing antibodies; thereby providing a determination of a level of protective immunity against MPV, based on a historic or contemporaneous correlation between amounts of MPV-neutralizing antibodies and levels of protective immunity against MPV. In particular embodiments, determining the amount of monkeypox virus (MPV)-neutralizing antibodies is by reference to a standard correlation between amounts of MPV-specific antibodies and amounts of MPV-neutralizing antibodies present in serum samples from previously vaccinated or infected individuals.

Dual, or parallel detection. Particularly preferred embodiments the SABRE-identified polypeptides and respective antibodies provide high-throughput dual (parallel) detection systems having utility for both direct detection of a particular pathogen, and for detecting immune response against the particular pathogen. Early during an infection, a pathogen will be present before a detectable immune response can be mounted. However, after an effective immune response is mounted (and/or disease symptoms arise), the pathogen sometimes becomes more difficult to detect, but the elicited immune response will remain for an extended period. The inventive dual-detection SABRE reagents provide for: (i) direct and specific detection of the pathogen using extremely specific monoclonal antibody reagents (i.e., antibodies specific the SABRE-identified immunodominant polypeptides); or (ii) specific detection of the immune response to the pathogen using the same unique pathogen-specific, SABRE-identified immunodominant polypeptides (e.g., by using the polypeptides/antigens/epitopes to coat ELISA plates or using other immunoassay methods).

Significantly, a clinician has the highest likelihood of making a positive diagnosis, regardless of the stage of disease or infection, by using both detection methods simultaneously (or contemporaneously), so as to enable consideration of both detection results in the diagnosis with respect to a particular subject (or sample). The diagnostic assays are rapid, high-throughput and suitable for ‘point-of-care’ implementations.

Particular embodiments provide a high-throughput method for parallel detection of both virus infection and immune response against the virus, comprising: obtaining a test serum sample from a test subject; detecting virus in the sample using a first immunologic assay based, at least in part, on use of at least one antibody reagent, or epitope-binding portion thereof, specific for a viral protein or polypeptide antigen; and detecting viral-specific antibodies in the sample using a second immunologic assay, based, at least in part, on use of at least one of the viral proteins or polypeptides, wherein at least one of the proteins or polypeptides used for detecting virus-specific antibodies is the cognate antigen of one of the antibody reagents, or epitope binding portions thereof.

In particular embodiments, the immunologic assay is selected from the group consisting of ELISA, immunoprecipitation, immunocytochemistry, immunoelectrophoresis, immunochemical methods, Western analysis, antigen-capture assays, antibody-capture assays, two-antibody sandwich assays, binder-ligand assays, agglutination assays, complement assays, and combinations thereof. In particular embodiments, a plurality of antibody reagents, or epitope-binding portions thereof, are used, and wherein a plurality of viral protein or polypeptide antigens are used. In particular embodiments, the plurality of antibody reagents, or epitope-binding portions thereof, and the plurality of viral protein or polypeptide antigens are cognate pairs. In particular embodiments, the virus is an orthopoxvirus. In particular embodiments, the orthopoxvirus is selected from the group consisting of smallpox, vaccinia and monkeypox.

In particularly preferred embodiments, the invention provides a high-throughput method for parallel detection of both monkeypox virus (MPV) infection and MPV-specific immune response, comprising: obtaining a test serum sample from a test subject; detecting MPV in the sample using a first immunologic assay based, at least in part, on use of at least one antibody reagent, or epitope-binding portion thereof, specific for an MPV protein or polypeptide antigen selected from the group consisting of D2L, N2R, N3R, B18R, B21R and epitope-bearing fragments of D2L, N2R, N3R, B18R and B21R; and detecting MPV-specific antibodies in the sample using a second immunologic assay, based, at least in part, on use of at least one of the MPV proteins or polypeptides, thereby providing for detection of both monkeypox virus (MPV) infection and MPV-specific immune response using the same serum sample.

In particular embodiments at least one of the proteins or polypeptides used for detecting MPV-specific antibodies is the cognate antigen of one of the antibody reagents, or epitope binding portions thereof. Preferably, the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20 (see also TABLE 2 herein below, and TABLES 4, 5 and 6). In particular embodiments, the first and second immunologic assay is, in each case; selected from the group consisting of ELISA, immunoprecipitation, immunocytochemistry, immunoelectrophoresis, immunochemical methods, Western analysis, antigen-capture assays, antibody-capture assays, two-antibody sandwich assays, binder-ligand assays, agglutination assays, complement assays, and combinations thereof. In particular embodiments, the antibody reagent is selected from the group consisting of a single-chain antibody, a monoclonal antibody, a chimeric antibody, a humanized antibody, and a Fab fragment. In particular embodiments, a plurality of antibody reagents, or epitope-binding portions thereof, are used, and wherein a plurality of MPV protein or polypeptide antigens are used. In particular embodiments, the plurality of antibody reagents, or epitope-binding portions thereof, and the plurality of MPV protein or polypeptide antigens are cognate pairs.

Therefore, the inventive SABRE platform provides benefits and applications at several levels, including the following four: First, the SABRE method yields the most immunodominant epitopes suitable for detecting an immune response against the pathogen (even at later stages of disease); Second, the SABRE method yields the most immunodominant epitopes suitable for development of diagnostic monoclonal antibodies; Third the SABRE provides for dual detection as described above, and fourth, the SABRE method yields the most immunodominant epitopes suitable for development of therapeutic monoclonal antibodies for treatment or prevention.

Arrays. Yet further embodiments provide an array of different Monkeypox virus proteins or polypeptides epitopes (oligopeptides) immobilized on a solid phase. The term “microarray” refers broadly to both ‘polypeptide microarrays’ and ‘polypeptide chip(s),’ and encompasses all art-recognized solid supports, and all art-recognized methods for synthesizing polypeptides on, or affixing polypeptides molecules thereto. The solid-phase surface may comprise, from among a variety of art-recognized materials, silicon, glass, polystyrene, aluminum, steel, iron, copper, nickel, silver, gold or cellulose. However, nitrocellulose as well as plastics such as nylon, which can exist in the form of pellets or also as resin matrices, may also be used.

It is also anticipated that the oligopeptides, or particular sequences thereof, may constitute all or part of an “virtual array” wherein the oligopeptides, or particular sequences thereof, are used, for example, as ‘specifiers’ as part of, or in combination with a diverse population of unique labeled oligopeptides to analyze a complex mixture of analytes. In such methods, enough labels are generated so that each antibody in the complex mixture (i.e., each analyte) can be uniquely bound by a unique label and thus be detected (e.g., each label may be directly counted, resulting in a digital read-out of each molecular species in the mixture).

Preferred embodiments provide an array comprising a plurality of different monkeypox virus (MPV) proteins or polypeptides coupled to a solid phase, wherein the MPV proteins or polypeptides are selected from the group consisting of of D2L, N2R, N3R, B18R, B21R and epitope-bearing fragments of D2L, N2R, N3R, B18R and B21R.

Preferably, the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20 (see also TABLE 2 herein below, and TABLES 4, 5 and 6). Preferably, the solid phase comprises a material selected from the group consisting of silicon, cellulose, glass, polystyrene, polyacrylamide, aluminum, steel, iron, copper, nickel, silver, gold and combinations thereof.

Protective Immunity against Orthopoxviruses

Particular preferred aspects of the present invention provide novel methods for detection/measurement of protective immunity against specific orthopoxviruses (e.g., smallpox, vaccinia and monkeypox).

There have been differing opinions with respect to what is required for full protective immunity against orthopoxviruses (Fenner et al., The pathogenesis, immunology, and pathology of smallpox and vaccinia; World Health Organization, Geneva, 1988). Two prospective studies (Mack et al., Am J Trop Med Hyg, 21:214-218, 1972; Sarkar et al., Bull. World Health Organ., 52:307-311, 1975), along with a study comparing antibody titers to survival during active smallpox infection (Downie & McCarthy, J. Hyg., 56:479-487, 1958), are consistent with a model in which high levels of neutralizing antibodies are at least associated with protective immunity against smallpox. Specifically, Mack et al. demonstrated that contacts of smallpox victims who had neutralizing titers of <1:32 were more susceptible to smallpox infection (3/15 (20%) contacts infected) than contacts with pre-existing antibody titers of ≧1:32 (0/127 (<1%) contacts infected). Likewise, Sarkar et al., in a smaller study, showed that 6/43 (14%) contacts with neutralizing titers of <1:20 contracted smallpox, whereas 0/13 contacts with titers ≧1:20 Contracted the disease.

Significantly, however, these studies do not prove, and have not been regarded in the art as indicating a determinative role for neutralizing antibodies in protective immunity, since high levels of antiviral antibodies may have been passively associated with, for example, higher underlying T cell memory. Moreover, and significantly, until the present invention, ELISA assays typically used for measurement of serum antibody levels, have been widely and dogmatically appreciated in the art as not having utility for determination of neutralizing antibodies and protective immunity.

The present invention represents a surprising departure from the long-standing art-recognized dogma that immunological (e.g., ELISA) assays have no utility for determination of protective immunity against orthopoxviruses.

According to preferred aspects of the present invention, and consistent with the EXAMPLES disclosed herein below, serum antibody levels are a useful biomarker of protective immunity, regardless of whether protection is mediated by B cells, T cells, or a combination of both antiviral immune mechanisms.

According to the present invention, an orthopoxvirus-specific immunoassay (e.g., ELISA) is used to detect or measure orthopoxvirus (e.g., smallpox, vaccinia, monkeypox)-specific serum antibodies. The serum antibody levels are, in turn, correlated with a level of neutralizing antibodies, thereby providing a determination of a level of protective immunity against the orthopoxvirus, based on a historic or contemporaneous correlation between amounts of orthopoxvirus-neutralizing antibodies and levels of protective immunity against the orthopoxvirus.

In particular embodiments, the correlation between orthopoxvirus-specific serum antibodies and neutralizing antibodies is established by quantifying the levels of orthopoxvirus-specific neutralizing antibodies in appropriate serum samples (e.g., vaccinated and unvaccinated individuals) using a corresponding orthopoxvirus plaque-reduction assay (e.g., to determine the serum dilution at which 50% of the infectious virus is/was neutralized (NT₅₀)).

The inventive assays are specific and sensitive, and have utility for reliably determining whether protective immunity exists against particular orthopoxviruses in particular individuals.

In preferred embodiments, specific anti-orthopoxvirus antibodies are detected by the inventive ELISA assays in collected serum samples as an indirect measurement of protective immunity, and prior exposure. In particular embodiments, the orthopoxviruses include, but are not limited to smallpox, monkeypox and vaccinia viruses. Additionally, because some antibodies raised against vaccinia are cross reactive with other orthopoxviruses, including smallpox and monkeypox, the inventive system enables medical practitioners to determine the likelihood that a patient maintains protective immunity to multiple orthopoxviruses for years or decades following vaccination with vaccinia. The diagnostic assays are rapid, high-throughput and suitable for ‘point-of-care’ implementations.

In particular embodiments, the orthopoxviruses include, but are not limited to smallpox, monkeypox and vaccinia viruses.

Preferred aspects provide a high-throughput method for detecting protective immunity against smallpox virus, comprising: obtaining a test serum sample from a test subject previously vaccinated with a vaccinia-based vaccine; detecting an amount of vaccinia virus-specific antibodies in the sample using an immunologic assay; and determining, based at least in part on the amount of vaccinia virus-specific antibodies, a corresponding amount of vaccinia virus-neutralizing antibodies; thereby providing a determination of a level of protective immunity against smallpox virus, based on a historic correlation between amounts of vaccinia virus-neutralizing antibodies and protective immunity against small pox virus.

In particular embodiments, determining the amount of vaccinia virus-neutralizing antibodies is by reference to a historic or contemporaneous correlation between amounts of vaccinia virus-specific antibodies and amounts of vaccinia virus-neutralizing antibodies present in serum samples from individuals previously vaccinated with a vaccinia-based vaccine. In particular embodiments, the vaccinia virus-neutralizing antibodies comprise vaccinia intramolecular mature virus (IMV)-neutralizing antibodies In particular embodiments, the immunologic assay comprises an assay selected from the group consisting of ELISA, immunoprecipitation, immunocytochemistry, immunoelectrophoresis, immunochemical methods, Western analysis, antigen-capture assays, antibody-capture assays, two-antibody sandwich assays, binder-ligand assays, agglutination assays, complement assays, and combinations thereof.

In particular embodiments, detecting an amount of vaccinia virus-specific antibodies in the sample using an immunologic assay, comprises forming immunocomplexes between the vaccinia virus-specific antibodies in the sample, and treated vaccinia virus, wherein the vaccinia virus has been treated with a peroxide agent prior to immunocomplex formation. In particular embodiments, the peroxide-treated vaccinia virus is immobilized on a surface prior to immumocomplex formation. In particular embodiments, treating of the vaccinia virus with a peroxide agent comprises treating with hydrogen peroxide. Preferably, during the treating, the hydrogen peroxide concentration is about 0.5% to about 10%, or about 1.0% to about 5%, or about 2% to about 4%, or about 3% (vol/vol).

### Immunologic Assays

According to the present invention, numerous art-recognized competitive and non-competitive protein binding immunoassays are used to detect and/or quantify antigens or antibodies (e.g., Harlow & Lane, Antibodies: A Laboratory Manual; Cold Spring Harbor Laboratory, New York 555-612, 1988). Such immunoassays can be qualitative or and/or quantitative, and include, but are not limited to antibody capture assays, antigen capture assays, and two-antibody sandwich assays, immunodiffusion, immunoelectrophoresis, immunochemical methods, binder-ligand assays, immunohistochemical techniques, agglutination and complement assays (e.g., Basic and Clinical Immunology, 217-262, Sites and Terr, eds., Appleton & Lange, Norwalk, Conn., 1991 which is incorporated herein by reference). Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

Antibody capture assays comprise immobilizing an antigen on a solid support, and contacting the immobilized antigen with an antibody—containing solution, whereby antigen-specific antibody, if present, binds to the immobilized antigen. The antibodies can be labeled or unlabeled. Antigen attachment to the solid support is typically non-covalent, but might in particular instances be covalent. After washing the support, antibody retained on the solid support is detected, or quantified by measuring the amount thereof. ELISA assays represent preferred embodiments of immunologic antibody capture assays as used herein. Competitive ELISA assays represent a preferred embodiment of antibody capture assay, wherein the antigen is bound to the solid support and two antibodies which bind the antigen (e.g., serum from a orthopoxvirus vaccine, and a monoclonal antibody of the present invention) are allowed to compete for binding of the antigen. The amount of monoclonal antibody bound is measured, and a determination made as to whether the serum contains anti-orthopoxvirus antigen antibodies. Such ELISAs can be used to indicate immunity to known protective epitopes in a vaccinee following vaccination.

Antigen capture assays comprise immobilizing an antibody to a solid support, and contacting the immobilized antibody with an antigen-containing solution, whereby antibody-specific antigen, if present, binds to the immobilized antibody. The antigens can be labeled or unlabeled. Antibody attachment to the solid support is typically non-covalent, but might in particular instances be covalent. After washing the support, antigen retained on the solid support is detected, or quantified by measuring the amount thereof.

Two-antibody sandwich assays (e.g., in the context of an antigen-capture assay) comprise initially immobilizing a first antigen-specific antibody on a solid support, followed by contacting the immobilized antibody with antigen-containing solution, washing the support, and subsequently detecting or quantifying the amount of bound antigen by contacting the immobilized antibody-antigen complexes with a second antigen-specific antibody, and measuring the amount of bound second antibody after washing.

Generally, immunoassays rely on labeled antigens, antibodies, or secondary reagents for detection. These proteins (antigens or antibodies) can be labeled with radioactive compounds, enzymes (e.g. peroxidase), biotin, or fluorochromes, etc. Enzyme-conjugated labels are particularly useful when radioactivity must be avoided, and provides for relatively rapid results. Biotin-coupled reagents are typically detected with labeled streptavidin. Streptavidin binds tightly and quickly to biotin and can be labeled with radioisotopes or enzymes. Fluorochromes, provide a very sensitive method of detection. Antibodies useful in these assays include, but are not limited to, monoclonal antibodies, polyclonal antibodies, affinity-purified polyclonal antibodies, and antigen or epitope-binding fragments of any of these. Labeling of antibodies or fragments thereof can be accomplished using a variety of art-recognized techniques (e.g., Kennedy et al., Clin. Chim. Acta., 70:1-31, 1976; Schurs et al., Clin. Chim Acta., 81:1-40, 1977; both incorporated by reference herein). Coupling techniques include, but are not limited to the glutaraldehyde, periodate method, dimaleimide and other methods.

ELISA. Enzyme-linked immunosorbent assay (ELISA) systems are widely recognized in the art, and are commonly used to detect antibodies in, for example, serum samples. For detection of antibodies in serum, a serum sample, or diluted serum sample, is applied to a surface (e.g. a well of a microliter plate, preferably ‘blocked’ to reduce non-specific protein binding) having immobilized antigens (epitope(s)) thereon. Serum antibodies specific for the immobilized epitope(s) bind with high affinity to the immobilized epitope(s) on the plate, and are retained after standard washes, whereas non-specific antibodies do not bind with high affinity, and are removed after standard washes.

Specifically bound antibody is detected, for example, by using enzyme-coupled anti-immunoglobulins and a chromogen (e.g., horseradish peroxidase-conjugated antibodies used in combination with hydrogen peroxide). The enzyme which is bound to the antibody will react with an appropriate substrate, preferably a chromogenic substrate, in such a manner as to produce a chemical moiety which can be detected, for example, by spectrophotometric, fluorimetirc or by visual means. Enzymes that can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

The detection can be accomplished by calorimetric methods that employ a chromogenic substrate for the enzyme. Detection may also be accomplished visually by comparison of the extent of enzymatic reaction with appropriate standards. Detection may also be accomplished using any of a variety of other immunoassays. For example, by radioactively labeling the antibodies or antibody fragments, it is possible to detect viral peptides peptides through the use of a radioimmunoassay (RIA). The radioactive isotope can be detected by such means as the use of a gamma counter or a scintillation counter or by autoradiography. It is also possible to label the antibody with a fluorescent compound. When the fluorescently labeled antibody is exposed to light of the proper wavelength, its presence can be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### Inventive ELISA

In particular embodiments, a inventive vaccinia-specific ELISA, as disclosed herein below under EXAMPLE III, is preformed essentially as previously described using a vaccinia-infected cell lysate (osmotic/freeze-thaw lysis) to coat 96-well flat-bottomed plates (Slifka & Ahmed, J. Immunol. Methods, 199:37-46, 1996)⁴⁸. However, and significantly, in departure from prior art ELISA technology, nether heat nor a classic protein denaturant (e.g., formaldehyde) is used to denature the vaccinia virus proteins prior to coating of the plates. Rather, in preferred embodiments for vaccinia-based ELISA, peroxide (e.g. hydrogen peroxide) is used to treat the vaccinia virus proteins (cell lysate) before coating the plates therewith. Preferably hydrogen peroxide is used to treat vaccinia virus at a concentration of at least 0.1%, at least 0.5%, at least 1.0%, and least 2% at least 3%, at least 5%, or at least 10%, but less than about 20% or 30%. Preferably the hydrogen peroxide concentration is in a range of about 0.5% to about 10%, or about 1.0% to about 5%, or about 2% to about 4%, or about 3%. Preferably the peroxide concentration is about 3%.

Significantly, in preferred aspects for vaccinia virus-based ELISA, substitution of peroxide (e.g., hydrogen peroxide) in place of heat or classic protein denaturants (e.g., formaldehyde) enables detection of anti-vaccinia serum antibody levels that are correlatable with a level of neutralizing antibodies, thereby providing a determination of a level of protective immunity against an orthopoxvirus (or cross-reactive orthopoxvirus), based on a historic or contemporaneous correlation between amounts of orthopoxvirus-neutralizing antibodies and levels of protective immunity against the orthopoxvirus:

### Neutralization Assays

Neutralization assays, as disclosed herein (see EXAMPLE III below), were performed following an optimized protocol similar to that previously described (Mack et al., Am. J. Trop. Med. Hyg., 21:214-218, 1972; Cutchins et al., J. Immunol., 85:275-283, 1960)^{8,50}.

### Generation and Production of Antibodies

Polyclonal or monoclonal antibodies to orthopoxvirus proteins and polypeptides or to epitope-bearing fragments thereof can be made for therapeutic, or diagnostic (e.g., immunoassays) use by any of a number of methods known in the art. By epitope reference is made to an antigenic determinant of a polypeptide. An epitope could comprise 3 amino acids in a spatial conformation which is unique to the epitope (methods of determining the spatial conformation of amino acids are known in the art, and include, for example, x-ray crystallography and 2 dimensional nuclear magnetic resonance). Generally an epitope consists of at least 5 such amino acids. The present invention encompasses epitopes and/or polypeptides recognized by antibodies of the present invention, along with conservative substitutions thereof, which are still recognized by the antibodies.

One approach for preparing antibodies to a protein is the selection and preparation of an amino acid sequence of all or part of the protein, chemically synthesizing the sequence and injecting it into an appropriate animal, usually a rabbit or a mouse.

Oligopeptides can be selected as candidates for the production of an antibody to orthopoxvirus proteins or polypeptides based upon the oligopeptides lying in hydrophilic regions, which are thus likely to be exposed in the mature protein.

Alternatively, proteins and polypeptides can be selected by the inventive SABRE method disclosed herein. Additionally, a combination of selection methods can be used.

Preferred proteins and polypeptides of the present invention are those of pathogenic viruses, such as orthopoxvirus proteins (e.g., smallpox, vaccinia and monkeypox). Preferably, they are of a strain of monkeypox virus. Preferably, they are a monkeypox virus (MPV) protein or polypeptide antigen selected from the group consisting of D2L, N2R, N3R, B18R, B21R and epitope-bearing fragments of D2L, N2R, N3R, B18R and B21R. Preferably, the proteins and polypeptides are selected from the group consisting of those listed in TABLEs 2, 4, 5 and 6herein below (SEQ ID NOS:1-29 and 30-44).

In particular embodiments, the monkeypox protein or polypeptide comprises at least one epitope of a sequence selected from the group consisting of SEQ ID NOS:1 (MPV D2L), 6 (MPV N2RR), 10 (N3R), 16 (B18R) and 20 (B21R), and epitope-bearing fragments of SEQ ID NOS:1 (MPV D2L), 6 (MPV N2R), 10 (MPV N3R), 16 (MPV B18R) and 20 (MPV B21R).

In particular embodiments, the monkeypox protein or polypeptide comprises at least one epitope of a sequence selected from the group consisting of SEQ ID NOS:2-5 (MPV D2L), 7-9 (MPV N2R), 11-15 (MPV N3R), 17-19 (MPV B18R) and 21-29 (MPV B21R), and epitope bearing fragments of SEQ ID NOS:2-5, 7-9, 11-15, 17-19, 21-29 and 30-44.

In particular embodiments, the monkeypox protein or polypeptide comprises at least one epitope of a sequence selected from the group consisting of SEQ ID NOS:10 (MPV N3R) and 20 (MPV B21R), and epitope-bearing fragments of SEQ ID NOS:10 and 20.

In particular embodiments, the monkeypox protein or polypeptide comprises at least one epitope of a sequence selected from the group consisting of SEQ ID NOS:11-15 (MPV N3R) and 21-29 (MPV B21R), and epitope bearing fragments of SEQ ID NOS: 11-15, 21-29 and 30-44.

In particular embodiments, the epitope comprises a sequence selected from the group consisting of SEQ ID NOS:15 (MPV N3R₁₅₇₋₁₇₆) and 27 (MPV B21R₇₂₉₋₇₄₈), and epitope-bearing fragments of SEQ ID NOS:15 and 27. Preferably, the epitope comprises a sequence selected from the group consisting of SEQ ID NO: 31 and epitope-bearing fragments of SEQ ID NO:31.

Preferred proteins and oligopeptides of the present invention are shown in TABLES 2, 4, 5 and 6 (under EXAMPLE IV, V and VI herein below).

Methods for preparation of the orthopoxvirus proteins or polypeptides, or of an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149, 1963 which is incorporated by reference) or the FMOC strategy on a Rapid Automated Multiple Peptide Synthesis system (E. I. du Pont de Nemours Company, Wilmington, Del.) (Caprino and Han, J Org Chem 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified orthopoxvirus proteins or polypeptides usually by ELISA or by bioassay based upon the ability to block the action of orthopoxvirus proteins or polypeptides. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating infectious disease. Thus, another aspect of the present invention provides for a method for preventing or treating diseases involving treatment of a subject with specific antibodies to orthopoxvirus proteins or polypeptides.

Specific antibodies, either polyclonal or monoclonal, to the orthopoxvirus proteins or polypeptides can be produced by any suitable method known in the art as discussed above. For example, murine or human monoclonal antibodies can be produced by hybridoma technology or, alternatively, the orthopoxvirus proteins or polypeptides, or an immunologically active fragment thereof, or an anti-idiotypic antibody, or fragment thereof can be administered to an animal to elicit the production of antibodies capable of recognizing and binding to the orthopoxvirus proteins or polypeptides. Such antibodies can be from any class of antibodies including, but not limited to IgG, IgA, IgM, IgD, and IgE or in the case of avian species, IgY and from any subclass of antibodies.

The present invention further provides for methods to detect the presence of the orthopoxvirus proteins or polypeptides in a sample obtained from a patient. As discussed above under “Immunologic Assays,” any method known in the art for detecting proteins can be used. Such methods include, but are not limited to immunodiffusion, immunoelectrophoresis, immunochemical methods, binder-ligand assays, immunohistochemical techniques, agglutination and complement assays. (for example, see Basic and Clinical Immunology, 217-262, Sites and Terr, eds., Appleton & Lange, Norwalk, Conn., 1991 which is incorporated by reference). Preferred are ELISA methods, including reacting antibodies with an epitope or epitopes of the orthopoxvirus proteins or polypeptides.

As provided herein, the compositions and methods for diagnosis/detection of viral infection, or the therapeutic methods of treatment or prevention provided herein, may utilize one or more antibodies used singularly, or in combination with other therapeutics to achieve the desired effects. Antibodies according to the present invention may be isolated from an animal producing the antibody as a result of either direct contact with an environmental antigen or immunization with the antigen. Alternatively, antibodies may be produced by recombinant DNA methodology using one of the antibody expression systems well known in the art (see, e.g., Harlow & Lane, Antibodies: A Laboratory Manual, Cold Spring Harbor Laboratory (1988). Such antibodies may include recombinant IgGs, chimeric fusion proteins having immunoglobulin derived sequences or “humanized” antibodies that may all be used according to the present inventive aspects. In addition to intact, full-length molecules, the term antibody also refers to fragments thereof (e.g., scFv, Fv, Fd, Fab, Fab′ and F(ab)′₂ fragments), or multimers or aggregates of intact molecules and/or fragments that bind to the inventive antigens (proteins/polypeptides/epitopes). These antibody fragments bind antigen and may be derivatized to exhibit structural features that facilitate clearance and uptake (e.g., by incorporation of galactose residues).

In particular embodiments antibodies are monoclonal antibodies, prepared essentially as described in Halenbeck et al. U.S. Pat. No. 5,491,065 (1997), incorporated herein by reference.

Additional embodiments comprise humanized monoclonal antibodies. The phrase “humanized antibody” refers to an antibody initially derived from a non-human antibody, typically a mouse monoclonal antibody. Alternatively, a humanized antibody may be derived from a chimeric antibody that retains or substantially retains the antigen binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are less immunogenic in humans than the parental mouse monoclonal antibodies, they can be used for the treatment of humans with far less risk of anaphylaxis. Thus, these antibodies may be preferred in therapeutic applications that involve in vivo administration to a human.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, for example, Jones et al., Nature 321:522-525, 1986; Morrison et al., Proc. Natl. Acad. Sci., U.S.A., 81:6851-6855, 1984; Morrison and Oi, Adv. Immunol., 44:65-92, 1988; Verhoeyer et al., Science 239:1534-1536, 1988; Padlan, Molec. Immun. 28:489-498, 1991; Padlan, Molec. Immunol. 31(3):169-217, 1994; and Kettleborough, C. A. et al., Protein Eng. 4(7):773-83, 1991, each of which is incorporated herein by reference.

The phrase “complementarity determining region” refers to amino acid sequences which together define the binding affinity and specificity of the natural Fv region of a native immunoglobulin binding site (see, e.g., Chothia et al., J. Mol. Biol. 196:901-917, 1987; Kabat et al., U.S. Dept. of Health and Human Services NM Publication No. 91-3242, 1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance (e.g., via Ashwell receptors) (see, e.g., U.S. Pat. Nos. 5,530,101 and 5,585,089, both incorporated herein by reference).

Humanized antibodies to the inventive proteins can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735; disclosing monoclonal antibodies against a variety of antigenic molecules including IL-6, TNFa, human CD4, L-selectin, gp39, and tetanus toxin. The monoclonal antibodies can be tested for the ability to inhibit or neutralize the biological activity or physiological effect of the corresponding protein or pathogenic agent (e.g., virus). WO 96/3373 discloses that monoclonal antibodies against IL-8, derived from immune cells of transgenic mice immunized with IL-8, blocked IL-8 induced functions of neutrophils. Human monoclonal antibodies with specificity for the antigen used to immunize transgenic animals are also disclosed in WO 96/34096. The antibodies of the present invention are said to be immuospecific, or specifically binding, if they bind to the viral antigen (protein/polypeptide/epitope) with a Kₐ of greater than or equal to about 10⁴ M⁻¹, preferably of greater than or equal to about 10⁵ M⁻¹, more preferably of greater than or equal to about 10⁶ M⁻¹, and still more preferably of greater than or equal to about 10⁷ M⁻¹. Such affinities may be readily determined using conventional techniques, such as by equilibrium dialysis; by using the BIAcore 2000 instrument, using general procedures outlined by the manufacturer; by radioimmunoassay using ¹²⁵I-labeled proteins; or by another method known to the skilled artisan. The affinity data may be analyzed, for example, by the method of Scatchard et al., Ann N.Y. Acad. Sci., 51:660, 1949. Thus, it will be apparent that preferred antibodies will exhibit a high degree of specificity for the viral antigen of interest, and will bind with substantially lower affinity to other molecules.

Preferably the anti-pathogenic antibodies of the present invention are monoclonal antibodies. More preferably, the antibodies are humanized monoclonal antibodies.

The present invention is further illustrated by reference to the EXAMPLES below. However, it should be noted that these EXAMPLES, like the embodiments described above, are illustrative and are not to be construed as restricting the enabled scope of the invention in any way.

### Example I

### CD4⁺ T Cell-Mediated Immune Responses were Evaluated in Volunteers Examined at 1 Month to 75 Years Post-Vaccination, and Significant CD4⁺ T Cell Responses were Detected as Late as 75 Years Post-Immunization

Quantification of virus-specific CD4⁺ T cell responses. Very little is known about the duration of vaccinia-specific T cell responses or what proportion of vaccinated individuals will maintain detectable levels of CD4⁺ and/or CD8⁺ T cell memory. To shed light on this fundamental question, the maintenance of virus-specific immunity after smallpox vaccination was analyzed by conducting a non-randomized, cross-sectional analysis of CD4⁺ T cell-mediated immune responses in volunteers examined at 1 month to 75 years post-vaccination. Although the frequency of virus-specific CD4⁺ T cells waned slowly over time, T cell responses in most subjects remained at levels within 1-2 orders of magnitude of those achieved at ≦7 years post-vaccination and could be detected as late as 75 years post-immunization.

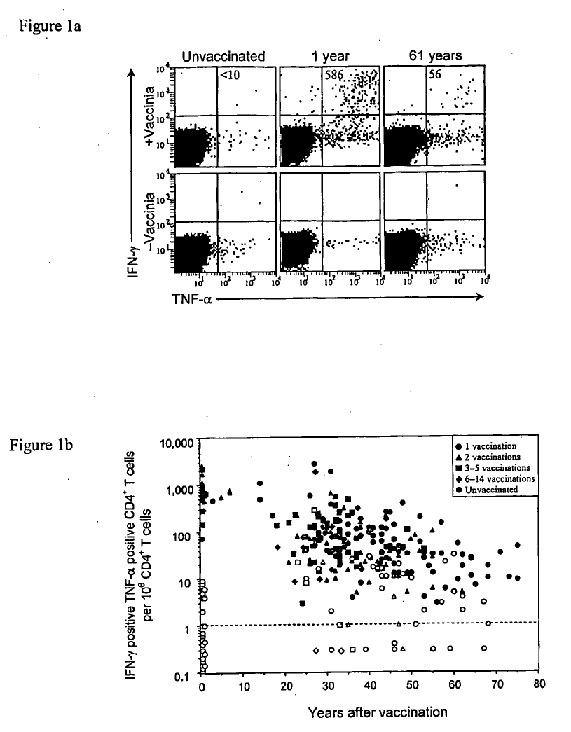

CD4⁺ T cell responses were quantified using a highly optimized intracellular cytokine staining (ICCS) protocol that detects vaccinia-specific T cells by virtue of their ability to produce potent antiviral factors including IFN-γ and TNF-α following exposure to vaccinia directly ex vivo (FIG. 1A{tilde over ())}. FIGS. 1A and 1B show the levels of Virus-specific CD4⁺ T cell memory following smallpox vaccination.

FIG. 1A illustrates a representative flow cytometry dotplot gated on CD4⁺CD8⁻ T cells showing the number of IFN-γ⁺ TNF-α⁺ events calculated per million CD4⁺ T cells (+Vaccinia) after background subtraction (−Vaccinia) in PBMC samples from an unvaccinated volunteer, or from volunteers analyzed at 1 or 61 years post-vaccination. After background subtraction (−Vaccinia), IFN-γ⁺TNF-α⁺ CD4⁺ T cells were below detection in the representative unvaccinated control (<10/10⁶ CD4⁺ T cells), but readily observed at 1-year post-vaccination (586/10⁶ CD4⁺ T cells) as well as at 61 years post-vaccination (56/10⁶ CD4⁺ T cells). In both of these latter cases, the number of IFN-γ⁺TNF-α⁺ events in the vaccinia-stimulated samples (+Vaccinia) was more than 10-fold higher than those observed in the unstimulated (−Vaccinia) controls cultured in parallel. Moreover, in 7 consecutive experiments, samples from the same volunteer at 1 year post-vaccination averaged 622±125 IFN-γ⁺TNF-α⁺ CD4⁺ T cells per million total CD4⁺ T cells, indicating that this is a highly reproducible assay.

Significantly, approximately 90% of IFN-γ⁺ vaccinia-specific CD4⁺ T cells co-expressed TNF-α, indicating that they maintained a “memory phenotype” of dual cytokine expression (Slifka & Whitton, J. Immunol., 164:208-216, 2000)¹¹. Subpopulations of IFN-γ⁺TNF-α⁻ and IFN-γ⁻TNF-α⁺ T cells were also observed in some, but not all, individuals (e.g., FIG. 1A). The most conservative estimates obtained by enumeration of functional T cells capable of dual IFN-γ and TNF-α production were relied on for quantification of the duration of CD4⁺ T cell memory.

FIG. 1B shows the quantification of virus-specific CD4⁺ T cells as a function of time post-vaccination. Following vaccination or revaccination, virus-specific CD4⁺ T cells were detected in 18/18 vaccinees at 27-34 d post-immunization (average=900/10⁶ CD4⁺ T cells) and then declined slowly with a half-life of 8 to 12 years (FIG. 1B and TABLE 1).

TABLE 1, below, shows the estimated survival of virus-specific T cell memory following smallpox vaccination. Interestingly, although multiple vaccinations are believed to provide maximum long-term protection (Nyerges et al., Acta Microbial. Acad. Sci., Hung. 19:63-68, 1972; el-Ad et al., J. Infect. Dis., 161:446-448, 1990)^{12,13}, repeated exposure to vaccinia did not greatly alter the magnitude (FIG. 1B), or the half-life of T cell memory (TABLE 1).

Significantly, although the frequency of virus-specific CD4⁺ T cells waned slowly over time, T cell responses in most subjects remained at levels within 1-2 orders of magnitude of those achieved at ≦7 years post-vaccination and could be detected as late as 75 years post-immunization.

TABLE 1
Estimated survival of virus-specific T cell memory following smallpox
vaccination.

| # | | | | |
|---|---|---|---|---|
| Vaccinations | % of volunteers with CD4⁺ T cell memory^{a} | | | T_{1/2} of |
| cells^{b} | 20-30 years^{c} | 31-50 years | 51-75 years | CD4⁺ T |
| 1 | 100% (16/16) | 89% (70/79) | 52% (23/44) | 10.6 (0-17)^{d} |
| 2 | 83% (10/12) | 78% (29/37) | 57% (4/7) | 8.3 (0-14.1) |
| 3-14 | 82% (23/28) | 91% (29/32) | N.D.^{e} | 12.4 (0-20.5) |
| # | | | | |
| Vaccinations | % of volunteers with CD8⁺ T cell memory | | | T_{1/2} of |
| cells | 20-30 years | 31-50 years | 51-75 years | CD8⁺ T |
| 1 | 50% (8/16) | 49% (39/79) | 50% (22/44) | 15.5 (0-27.1) |
| 2 | 42% (5/12) | 38% (14/37) | 57% (4/7) | 8.1 (0-16.9) |
| 3-14 | 46% (13/28) | 50% (16/32) | N.D. | 9.0 (0-18.1) |

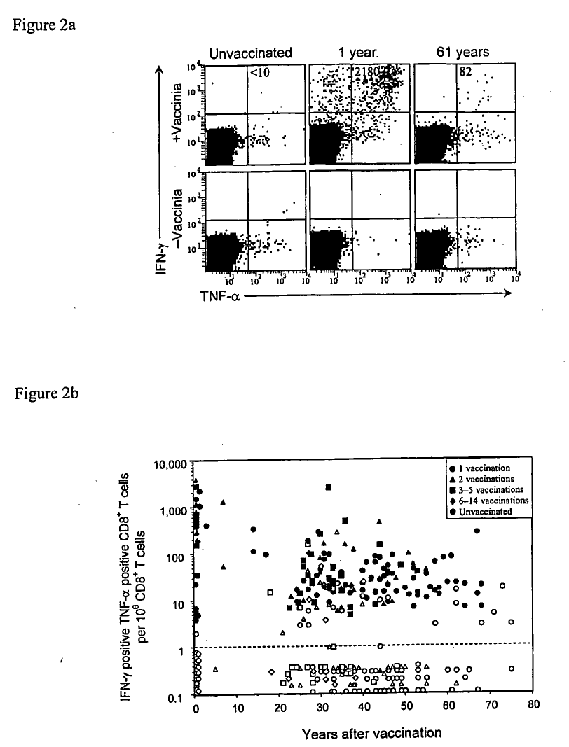

^{a}Percentage of volunteers with vaccinia-specific T cell memory was based on the proportion of immunized participants with >10 IFN-γ⁺TNF-α⁺ T cells/10⁶ CD4⁺ or CD8⁺ T cells, respectively. This cut-off point provided 100% sensitivity at 1-month post-vaccination/revaccination and 92-96% specificity, based on the vaccinia-induced IFN-γ response in T cells from unvaccinated volunteers.
^{b}Estimated T_{1/2} in years was based on linear regression analysis using the data from FIGS. 1 and 2.
^{c}Years after the last smallpox vaccination.
^{d}95% Confidence Intervals.
^{e}N.D., Not Determined.

### Example II

### CD8⁺ T Cell-Mediated Immune Responses were Evaluated in Volunteers Examined at 1 Month to 75 Years Post-Vaccination, and Significant CD8⁺ T Cell Responses were Detected as Late as 75 Years Post-Immunization

Quantification of virus-specific CD8⁺ T cell responses. The maintenance of virus-specific immunity after smallpox vaccination was also analyzed by conducting a non-randomized, cross-sectional analysis of antiviral antibody and CD8⁺ T cell-mediated immune responses in volunteers examined at 1 month to 75 years post-vaccination. Robust CD8⁺ T cell responses were identified (FIG. 2B), and similar to CD4⁺ T cells (FIG. 1B), CD8⁺ T cells declined slowly with a half-life of 8 to 15 years (TABLE 1).

Antiviral CD8⁺ T cell responses were quantified by ICCS following direct ex vivo stimulation with vaccinia-infected cells (FIG. 2A).

FIG. 2 shows the levels of virus-specific CD8⁺ T cell memory following smallpox vaccination. FIG. 2A shows a representative flow cytometry dotplot gated on CD8⁺CD4⁻ T cells showing the number of IFN-γ⁺ TNF-α⁺ events calculated per million CD8⁺ T cells (+Vaccinia) after background subtraction (−Vaccinia) in PBMC samples from an unvaccinated volunteer, or from volunteers analyzed at 1 or 61 years post-vaccination. FIG. 2B shows the quantitation of virus-specific CD8⁺ T cells as a function of time post-vaccination.

Although a recent study has identified two HLA-A*0201-restricted T cell epitopes (Terajima et al., J. Exp. Med., 197:927-932, 2003)¹⁴, these epitopes measure only a subpopulation of the total T cell response (supra)¹⁴ and therefore live virus was used to stimulate T cells in this cross-sectional study so that the global antiviral CD8⁺ T cell response could be identified, irrespective of the HLA type of the donor (Speller & Warren, J. Immunol. Methods, 262:167-180, 2002)¹⁵, and to allow side-by-side comparisons with CD4⁺ T cell responses (for which no epitopes have yet been mapped).

The majority of IFN-γ⁺CD8⁺ T cells co-expressed TNF-α and again we used dual cytokine production as the functional criteria for quantitating virus-specific T cell memory. Samples from one volunteer (1 year post-vaccination) averaged 2,215±325 IFN-γ⁺TNF-α⁺ CD8⁺ T cells per million CD8⁺ T cells in 7 consecutive experiments. At 27-34 d post-vaccination or revaccination, robust CD8⁺ T cell responses (average=870/10⁶ CD8⁺ T cells) were identified in 18/18 vaccinees (FIG. 2B). Similar to CD4⁺ T cells (FIG. 1B), CD8⁺ T cells declined slowly with a half-life of 8 to 15 years (TABLE 1). Comparison of CD8⁺ T cell levels following booster vaccination did not reveal any substantial improvements in long-term T cell memory above that observed following a single vaccination (FIG. 2B and TABLE 1).

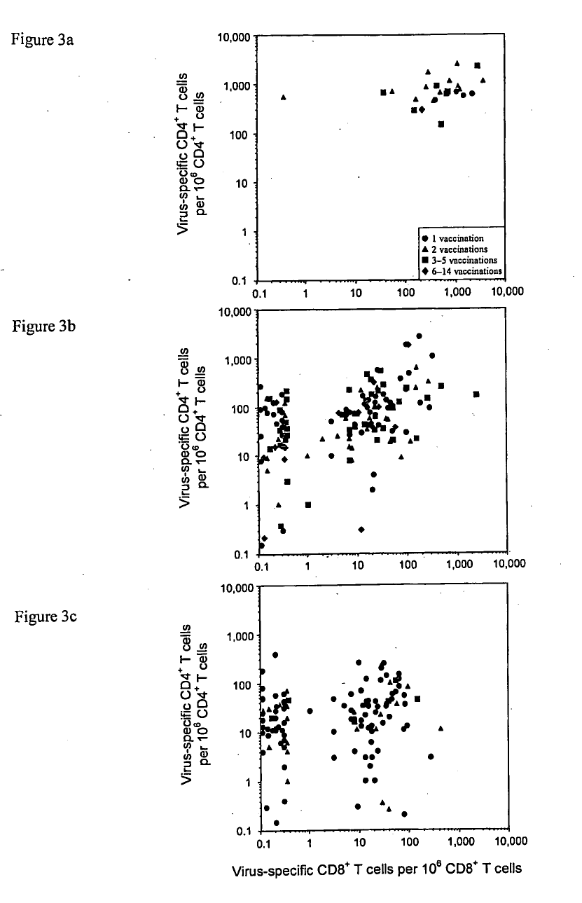

Direct comparisons between virus-specific CD4⁺ and CD8⁺ T cell levels within individual vaccinees revealed dynamic and independently regulated changes in T cell memory over time (FIG. 3).

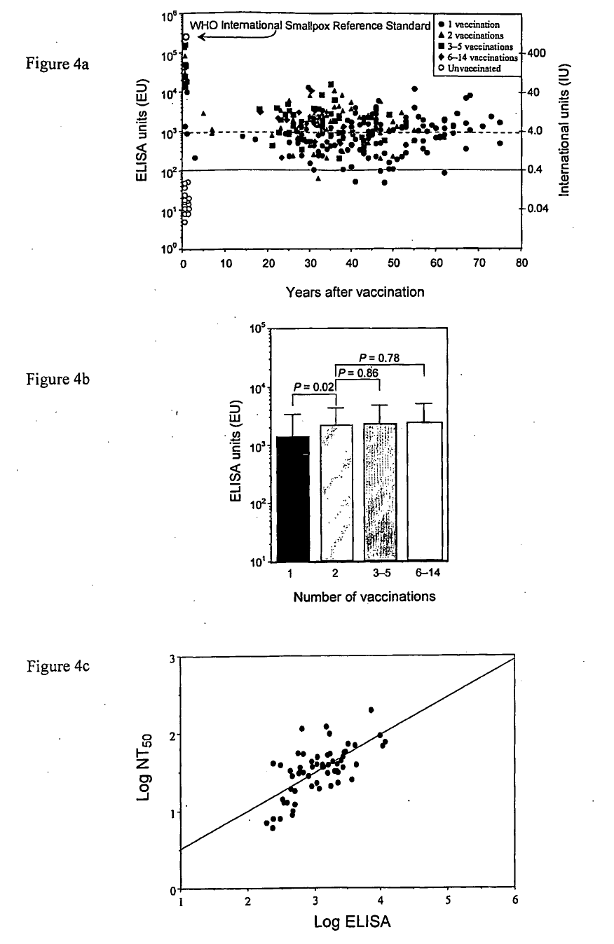

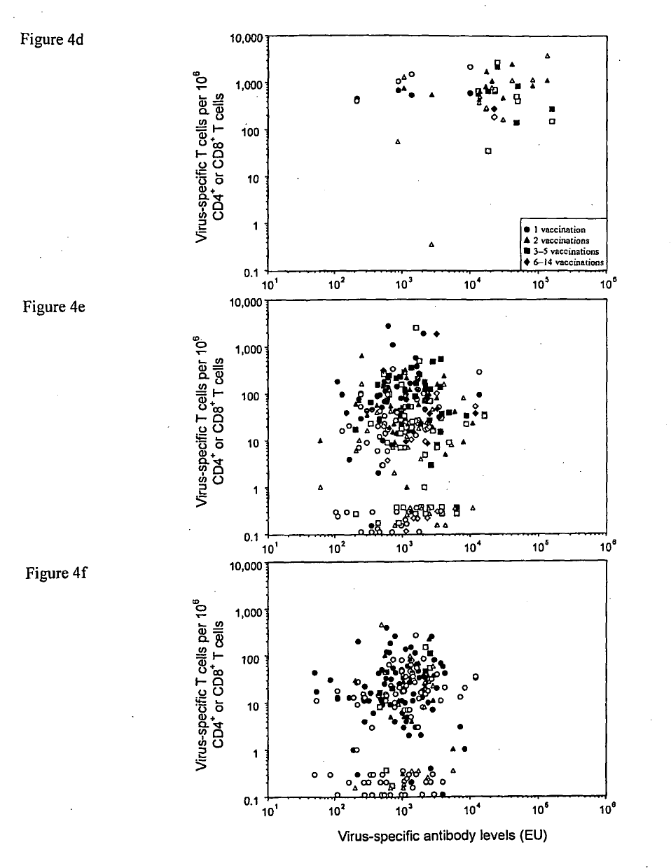

FIG. 3 shows the relationship between vaccinia-specific CD4⁺ and CD8⁺ T cell memory over time. Comparisons were made between the number of antiviral CD4⁺ and CD8⁺ T cells from the same individual. FIG. 3A shows 1 month to 7 years post-vaccination, whereas FIG. 4B shows 14 to 40 years post-vaccination, and FIG. 4C shows 41 to 75 years post-vaccination.

At early time points ranging from 27-days to 7-years post-vaccination, nearly all of the volunteers possessed strong CD4⁺ and CD8⁺ T cell responses (FIG. 3A).

At later time points, examined between 14-40 years post-vaccination (FIG. 3B) or 41-75 years post-vaccination (FIG. 3C), many individuals still maintained both CD4⁺ and CD8⁺ T cell memory (albeit at lower levels than earlier time points observed in FIG. 3A), but other individuals preferentially lost CD8⁺ T cell memory while leaving the antiviral CD4⁺ T cell compartment intact.

In rare cases, CD8⁺ T cell responses remained elevated while. CD4⁺ T cell responses dropped to below detection. Further studies will be necessary to determine why virus-specific CD8⁺ T cells, or in some cases, CD4⁺ T cells are disproportionably lost over prolonged periods of time, but the overall shift in T cell memory appears to reflect the antiviral CD4⁺ and CD8⁺ T cell survival rates (TABLE 1).

### EXAMPLE III

### The Duration of Antiviral Antibodies were Examined in Volunteers Examined at 1 Month to 75 Years Post-Vaccination, and Vaccinia-Specific Serum Antibody Levels were found to be Remarkably Stable between 1 Year to 75 Years Post-Vaccination

Duration of antiviral antibody production. The maintenance of virus-specific immunity after smallpox vaccination was analyzed by conducting a non-randomized, cross-sectional analysis of antiviral antibodies in volunteers examined at 1 month to 75 years post-vaccination. In striking contrast to vaccinia-specific T cell memory which declined steadily over time (FIGS. 1 and 2), vaccinia-specific serum antibody levels were remarkably stable between 1 year to 75 years post-vaccination (FIG. 4).

Vaccinia-specific neutralizing antibody titers have been the cardinal feature used to estimate the level of immunity afforded by smallpox vaccination (Fenner et al. in The pathogenesis, immunology, and pathology of smallpox and vaccinia, World Health Organization, Geneva, 1988; Downie & McCarthy, J. Hyg., 56:479-487, 1958; McCarthy & Downie, J. Hyg., 56:466-478, 1958; Stienlauf et al., Vaccine, 17:201-204, 1999; CDC, MMWR, 50:1-25, 2001; Frey et al., JAMA, 289:3295-3299, 2003)^{7,10,16-19}. To examine this issue in more detail, a sensitive, reproducible, and validated vaccinia-specific ELISA was developed for high-throughput analysis of humoral immunity following smallpox vaccination.

Inventive ELISA assay. The ELISA for detection of anti-vaccinia virus antibodies was preformed essentially as previously described using a vaccinia-infected cell lysate (osmotic/freeze-thaw lysis) to coat 96-well flat-bottomed plates (Slifka & Ahmed, J. Immunol Methods, 199:37-46, 1996)⁴⁸. Significantly however, and in departure from prior art ELISA technology, nether heat nor a classic protein denaturant (e.g., formaldehyde) was used to denature the vaccinia virus proteins prior to coating of the plates. Rather, peroxide (e.g. hydrogen peroxide) was used to treat the vaccinia virus proteins (cell lysate) before coating the plates therewith. Preferably peroxide is used to treat vaccinia virus-containing solutions at a concentration of at least 0.5%, at least 1.0%, and least 2% at least 3%, at least 5%, or at least 10%. Preferably the peroxide concentration is in a range of about 0.5% to about 10%, or about 1.0% to about 5%, or about 2% to about 4%, or about 3%. Preferably the peroxide concentration is about 3%. The data in this exemplary analysis was obtained the treating the vaccinia virus in 3% hydrogen peroxide prior to coating plates therewith.

According to the present invention, substitution of peroxide (e.g., hydrogen peroxide) in place of heat or classic protein denaturants (e.g., formaldehyde) enables detection of anti-vaccinia serum antibody levels that are correlatable with a level of neutralizing antibodies, thereby providing a determination of a level of protective immunity against an orthopoxvirus, based on a historic or contemporaneous correlation between amounts of orthopoxvirus-neutralizing antibodies and levels of protective immunity against the orthopoxvirus.

Serial 3-fold dilutions of serum were incubated on pre-blocked ELISA plates for 1 h, washed, incubated with mouse anti-human IgG-HRP (clone G18-145, Pharmingen), washed, detection reagents added, and samples analyzed on a VERSAₘₐₓ™ ELISA plate reader (Molecular Devices). The WHO International Standard for anti-smallpox serum (Anderson & Skegg, Bull. World Health Organ., 42:515-523, 1970)⁴⁹ was used to calibrate antiviral IgG measured by ELISA and an internal positive control was included on every plate in order to normalize ELISA values between plates and between assays performed on different days. Antibody titers were determined by logarithmic transformation of the linear portion of the curve with 0.1 OD units used as the endpoint and conversion performed on final values.

FIG. 4 shows long-lived antiviral antibody responses induced by smallpox vaccination. FIG. 4A shows the quantitation of vaccinia-specific antibody responses by ELISA. FIG. 4B shows the levels of vaccinia-specific antibody titers (1 to 75 years post-vaccination) compared to the total number of vaccinations received. FIG. 4C shows the correlation between virus-specific antibody titers determined by ELISA and by neutralizing assays was determined by linear regression analysis after plotting the log values obtained from serum samples of volunteers vaccinated one or two times against smallpox. The slope of the line was defined as Log NT₅₀=0.056+0.487 Log ELISA. R²=0.450, P<0.0001. The relationship between virus-specific CD4⁺ (closed symbols) or CD8⁺ (open symbols) T cells (per million CD4⁺ or CD8⁺ T cells, respectively) with virus-specific antibody titers was determined at 1 month to 7 years post-vaccination (CD4; P=0.67, CD8; P=0.39) as shown FIG. 4D 14 years to 40 years post-vaccination (CD4; P=0.72, CD8; P=0.89), as shown in FIG. 4E, or 41 years to 75 years post-vaccination (CD4; P=0.77, CD8; P=0.06*) as shown in FIG. 4F.

Using 100 ELISA Units (EU) as the lowest titer considered to be positive, 100% specificity (0/26 unvaccinated controls scored ≧100 EU) and 98% sensitivity (288/293 samples from volunteers vaccinated against smallpox scored ≧100 EU) was observed. One representative positive control (scoring 644 EU) was repeated >40 times and varied by <12% within a single assay and varied by <18% between assays, with 0% false-negative results. Likewise, a representative negative control sample from an unvaccinated volunteer was repeated in >40 assays and in each case scored <50 EU, with 0% false-positive results.

In striking contrast to vaccinia-specific T cell memory which declined steadily over time (FIGS. 1 and 2), vaccinia-specific serum antibody levels were remarkably stable between 1 year to 75 years post-vaccination and we were unable to determine a half-life of antibody decay. Comparison of antiviral antibody titers elicited by one or more vaccinations revealed a very small (<2-fold), but statistically significant increase in the mean level of antibody produced after 2 vaccinations in comparison with only 1 vaccination (P=0.02, FIG. 4B). However, additional vaccinations ranging from 3-5 or as many as 6-14 immunizations did not result in any further increases in long-term antibody production. This indicates that booster vaccination may increase a previously suboptimal antibody response, but is unlikely to induce prolonged synthesis of higher antibody levels above a certain threshold level.

ELISA assays do not directly measure levels of neutralizing antibodies and must therefore be validated side-by-side with neutralizing assays in order for them to be useful as a means of quantitating biologically relevant antibody levels. By performing neutralizing assays in essentially the same manner as that described in previous studies in which an experimental value for protective immunity was defined (NT₅₀≧1:32) (Mack et al., supra)⁸, the data was directly related to historical findings that can not be repeated now that natural smallpox is extinct.

Specifically, several 3-fold dilutions (beginning at 1:4 or 1:12) of heat-inactivated serum were incubated with vaccinia (˜100 plaque forming units) for 2 h at 37° C. before incubating the virus with Vero cells for 1-h, overlaying with 0.5% agarose and incubating for 3.5 days to allow plaque formation. Cells were fixed with 75% methanol, 25% acetic acid, and after removing the agarose, plaques were visualized by staining with 0.1% crystal violet in PBS containing 0.2% formaldehyde. The neutralization titer (NT₅₀) was defined as the reciprocal of the serum dilution required for 50% reduction in vaccinia plaques. Logarithmic transformation of the data was used to calculate the titer and conversion was done on final values, excluding those in which ≧85% neutralization occurred. Antibodies against the extracellular enveloped virus (EEV) are highly protective in vivo (Galmiche et al., Virology, 254:71-80, 1999)³⁰ but so are antibodies against IMV (Czerny & Mahnel, J. Gen. Virol., 71:2341-2352, 1990; Ramirez et al., J. Gent. Virol., 83:1059-1067, 2002)^{29,31} and we chose IMV for our neutralization studies because there is a precedent for protective immunity against smallpox if the individuals have pre-existing neutralizing antibody titers (against IMV) that are ≧1:20 (Sarkar et al., Bull. World Health Organ., 52:307-311, 1975)⁹ or ≧1:32 (Mack et al., Am. J. Trop. Med. Hyg., 21:214-218, 1972)⁸. EEV would be unsuitable for these assays because we would not be able to compare our results to these historical values.

Significantly, a direct linear relationship (P<0.0001) was observed between neutralizing titers and the levels of virus-specific antibodies quantitated by the inventive ELISA (FIG. 4C). Based on this analysis, a NT₅₀ of 1:32 equals 944 EU (dashed line, FIG. 4A) and indicates that ˜50% of volunteers at >20 years after a single vaccination have neutralizing antibody titers of ≧1:32. Neutralizing antibodies were below detection (NT₅₀<1:4) in 16/16 samples from unvaccinated volunteers (data not shown).

An important point to consider is whether or not high antibody responses are correlated with increased levels of T cell memory, since this would shed new light on whether high neutralizing antibody titers were directly involved with protective immunity against smallpox or whether they are simply a surrogate marker indicative of increased antiviral T cell responsiveness. Therefore, antiviral T cell responses were compared to their accompanying antibody levels in individuals who had been vaccinated ≦7 years previously (i.e., a cohort similar to Mack et al., (supra)⁸) (FIG. 4D) as well as in individuals vaccinated 14-40 years ago, or 41-75 years ago (i.e., cohorts similar to contemporary populations) (FIGS. 4E and 4F).

Significantly, no correlation was observed between virus-specific T cell levels and antibody titers at early or late time points, thus indicating that humoral and cellular immunity are independently regulated. Because the early cohort (≦7 years post-vaccination) is reasonably comparable to the smallpox contacts examined by Mack et al., (supra)⁸, the results indicate that high neutralizing antibody titers are still an effective biomarker of protective immunity, but not necessarily indicative of enhanced T cell memory.

Significantly, this also indicates that high neutralizing antibody levels have a more direct role in protective immunity against smallpox than previously realized.

### Example IV

### Application of the Inventive SABRE Platform; Monkeypox Virus-Specific Immune Response was Detected

The inventive SABRE (systematic analysis of biologically relevant epitopes) platform, as described herein above and as exemplified in this EXAMPLE, enables rapid and effective mapping/identification of biologically relevant (e.g., immunodominant) polypeptide epitopes suitable for diagnostic and/or therapeutic applications.

Using SABRE, a representative serological assay was developed that can be used to determine whether or not a person has been infected with the monkeypox virus, a dangerous orthopoxvirus on the U.S. government's Select Agent list, and a possible pathogen that might be used for bioterrorism. Additionally, a dual assay for determining both virus infection, and virus-specific immune response is provided. Furthermore, vaccines agents are provided, based on the SABRE-identified antigens (proteins/polypeptides/epitopes).

Specifically, using published sequences of several strains of vaccinia, monkeypox (MPV), cowpox, and Variola (smallpox), monkeypox-specific genes were identified that are not encoded in the genome of vaccinia—the most common poxvirus that, for example, Americans are likely to have pre-existing immunity against. Overlapping peptide reagents were ordered and obtained (from Mimotopes) that spanned the entire protein of several of these genes (e.g., MPV genes: D2L, B18R, N2R and N3R), as well as to several likely candidates in the MPV B21R gene.

ELISA plates were coated with individual peptides and then tested the reactivity of serum samples from subjects with verified MPV infections, possible sub-clinical MPV infections, and negative controls including subjects recently immunized with vaccinia or subjects that have no known exposure to orthopoxvirus infections.

Based on this analysis, several unique peptide epitopes were identified that are immunogenic and recognized by MPV convalescent serum (5/5=positive), but not by negative control serum samples from subjects that have not been infected with MPV (0/4=positive). The preferred immunogenic polypeptides are summarized in TABLE 2 below.

Preferably, the monkeypox protein or polypeptide comprises at least one epitope of a sequence selected from the group consisting of SEQ ID NOS:1 (MPV D2L), 6 (MPV N2R), 10 (MPV N3R), 16 (MPV B18R) and 20 (MPV B21R), and epitope-bearing fragments of SEQ ID NOS:1 (MPV D2L), 6 (MPV N2R), 10 (MPV N3R), 16 (MPV B18R) and 20 (MPV B21R).

More preferably, the monkeypox polypeptide comprises at least one epitope of a sequence selected from the group consisting of SEQ ID NOS:2-5 (MPV D2L), 7-9 (MPV N2R), 11-15 (MPV N3R), 17-19 (MPV B18R) and 21-29 (MPV B21R), and epitope bearing fragments of SEQ ID NOS:2-5, 7-9, 11-15, 17-19 and 21-29.

More preferably, the monkeypox protein or polypeptide comprises at least one epitope of a sequence selected from the group consisting of SEQ ID NOS:10 (MPV N3R) and 20 (MPV B21R), and epitope-bearing fragments of SEQ ID NOS:10 and 20.

More preferably, the monkeypox protein or polypeptide comprises at least one epitope of a sequence selected from the group consisting of SEQ ID NOS:11-15 (MPV N3R) and 21-29 (MPV B21R), and epitope bearing fragments of SEQ ID NOS: 11-15 and 21-29.

More preferably, the epitope comprises a sequence selected from the group consisting to of SEQ ID NOS:15 (MPV N3R₁₅₇₋₁₇₆) and 27 (MPV B21R₇₂₉₋₇₄₈), and epitope-bearing fragments of SEQ ID NOS:15 and 27.

According to aspects of the present invention, these peptides are used in diagnostic kits and assays to detect virus immune response (e.g., virus-specific serum antibodies).

### Dual Determination of Infection and Monkeypox Virus-Specific Immunity

As described above, aspects of the present invention provide methods to identify monkeypox-specific immune response by screening the serum of subject for monkeypox-specific antibody. The window for such screening is very broad, because people continue to make antibodies to orthopoxviruses for decades. Nonetheless, such assays may not be sensitive enough to identify infected subjects at very early time points of infection, especially if an immune response has not yet been mounted.

According to the present invention, a Dual Detection System (DDS) overcomes this limitation. Preferably, the DDS is used to simultaneously (in parallel) identify either the orthopoxvirus (e.g., monkeypox), or the immune response against the virus (e.g., monkeypox-specific antibody).

For example, monkeypox-specific antibody responses, as describe above, are identified by initially screening a library of monkeypox peptides to identify peptides only recognized by serum samples from monkeypox infected patients. Monoclonal antibodies (e.g., mouse) are then developed against the unique peptide sequences identified in the infected serum screen. These antibodies have utility to detect the monkeypox virus, based on the fact that these peptides are very specific and non-cross-reactive.

Kits. According to preferred aspects of the invention, screened biologically relevant peptides are used, for example, as part of a ‘dip-stick’ kit to detect orthopoxvirus-specific serum antibodies by about 6 to 10 days after infection, and as late as 75-years after infection.

Additionally, because the highest virus titers are likely to precede the strongest antibody responses, the respective (cognate) monkeypox-specific monoclonal antibody are used, for example, as part of a ‘dip-stick’ kit to detect orthopoxvirus infection.

Therefore, the inventive dual assay approach broadens the window of detection to include the first signs of clinical symptoms. The DDS approach allows the broadest, yet highly specific identification of the pathogen of interest. By detecting the virus directly, positive results are obtained at early time points, before antibody responses have had time to be mounted. By detecting the virus-specific antibodies in parallel, positive results are obtained even in people that are recovering (or have recovered) from the infection (i.e., where there is no virus to find) or had such low virus titers to begin with that they scored negative by the direct virus-detection approach.

TABLE 2

Summary of exemplary monkeypox virus proteins, along with exemplary
preferred polypeptides thereof.

| Preferred Monkepox | Accession | Preferred | | |
|---|---|---|---|---|
| Virus Protein | Number | Polypeptide | Polypeptide Sequence | SEQ ID NO: |
| D2L | AAL40463 | | | SEQ ID NO: 1 |
| D2L | | 1-20 | MYYANICLDFDNNVYTVKDK | SEQ ID NO: 2 |
| D2L | | 11-30 | DNNVYTVKDKNYTNAVIEYP | SEQ ID NO: 3 |
| D2L | | 21-40 | NYTNAVIEYPVVCNFRRYSE | SEQ ID NO: 4 |
| D2L | | 31-50 | VVCNFRRYSESDSDVDDRAE | SEQ ID NO: 5 |
| N2R | NP_536613 | | | SEQ ID NO: 6 |
| N2R | | 1-20 | MQYLNETDNLGNTVLHTHIF | SEQ ID NO: 7 |
| N2R | | 21-40 | LDYISLKICKRYISHKYPLC | SEQ ID NO: 8 |
| N2R | | 31-50 | RYISHKYPLCNIINGYIDNT | SEQ ID NO: 9 |
| N3R | NP_536614 | | | SEQ ID NO: 10 |
| N3R | | 21-40 | CHKLVHYFNLKINGSDITNT | SEQ ID NO: 11 |
| N3R | | 91-110 | SSQYEELEYYYSCDYTNNRP | SEQ ID NO: 12 |
| N3R | | 111-130 | TIKQHYFYNGEEYTEIDRSK | SEQ ID NO: 13 |
| N3R | | 151-170 | DSEDCIIYLRSLVRRMEDSN | SEQ ID NO: 14 |
| N3R | | 157-176 | IYLRSLVRRMEDSNKNSKKT | SEQ ID NO: 15 |
| B18R | NP_536606 | | | SEQ ID NO: 16 |
| B18R | | 31-50 | YFKTMFTTPMIARDLATRVN | SEQ ID NO: 17 |
| B18R | | 41-60 | IARDLATRVNLQMFDKMPSK | SEQ ID NO: 18 |
| B18R | | 51-70 | LQMFDKMPSKILYSTYTIGI | SEQ ID NO: 19 |
| B21R | NP_536609 | | | SEQ ID NO: 20 |
| B21R | | 150-169 | PLPTSAVPYDQRSNNNVSTI | SEQ ID NO: 21 |
| B21R | | 195-214 | DTVDNNTMVDDETSDNNTLH | SEQ ID NO: 22 |
| B21R | | 362-381 | IRNSVSTTNSRKRRDLNGEF | SEQ ID NO: 23 |
| B21R | | 669-688 | TRKGATRRRPRRPTNDGLQS | SEQ ID NO: 24 |
| B21R | | 684-703 | DGLQSPNPPLRNPLPQHDDY | SEQ ID NO: 25 |
| B21R | | 699-718 | QHDDYSPPQVHRPPTLPPKP | SEQ ID NO: 26 |
| B21R | | 729-748 | PVGQLPPPIDQPDKGFSKFV | SEQ ID NO: 27 |
| B21R | | 805-824 | KNNVPVIGNKHSKKYTSTMS | SEQ ID NO: 28 |
| B21R | | 842-861 | TRSTTLSRKDQMSKEEKIFE | SEQ ID NO: 29 |

### Example V

### Inventive Assays were Used to Show that Cross-Protective Antiviral Immunity Against West African Monkeypox can be Maintained for Decades After Smallpox Vaccination

This Example shows, according to particular aspects, independent and internally validated diagnostic approaches with ≧95% sensitivity and ≧90% specificity for detecting clinical monkeypox infection. Applicants detected, inter alia, three previously unreported cases of monkeypox in pre-immune individuals at 13, 29, and 48 years post-smallpox vaccination who were unaware that they had been infected because they were spared any recognizable disease symptoms. Together, this shows that the U.S. monkeypox outbreak was larger than previously realized and more importantly, indicates that cross-protective antiviral immunity against West African monkeypox can be maintained for decades after smallpox vaccination.

Rationale. Approximately 50% of the U.S. population has received smallpox vaccinations before routine immunization ceased in 1972 (civilians) or 1990 (military personnel). There is a question as to whether any potential residual immunity would translate into full protection against the onset of orthopoxvirus-induced disease. The U.S. monkeypox outbreak of 2003 provided the opportunity to examine this critical issue.

Following hospitalization of the first monkeypox victim, ˜2 weeks elapsed before the outbreak was identified by local health officials and the CDC⁹. The current diagnostic algorithm for detection of smallpox¹⁰ was apparently not utilized since there were several instances of febrile patients with smallpox-like lesions who were misdiagnosed and discharged from the hospital (11). Similar failures in diagnosis occurred during a recent smallpox outbreak drill (12). If the monkeypox outbreak had actually been smallpox, the outcome might have been very different, with secondary infections and further spread likely to have occurred during the prolonged period needed to identify the outbreak. Current diagnostic tests, which aim to diagnose an acute infection, rely on the presence of virus (including virus-specific PCR; polymerase chain reaction) and thus approximately half of the 71 reported monkeypox cases remain unconfirmed (9) because the results have scored negative or equivocal. Moreover, there is a critical need for better diagnostics for investigating monkeypox outbreaks in Africa (13). One objective of this study was to develop novel and improved diagnostic methods for identifying rare orthopoxvirus infections.

Applicants have demonstrated herein that antiviral antibody and T cell responses could be maintained for up to 75 years after smallpox vaccination (see also 14). Since ˜50% of the U.S. has been vaccinated against smallpox, it is critical to determine whether or not the antiviral immunity identified in these studies might translate into protective immunity. The 2003 monkeypox outbreak provided a natural virus-challenge experiment that offers insight into this important question. The results presented herein identify three previously unreported cases of clinically inapparent monkeypox infections in which individuals who received the smallpox vaccine 13-48 years previously still maintained fully protective cross-reactive immunity and were spared any observable disease symptoms after exposure to an infectious dose of a West African strain of monkeypox. According to particular aspects, these individuals maintained protective cross-reactive immunity.

### Methods:

Subjects. Recruitment of adult volunteers (n=44) was conducted in the state of Wisconsin since this was the epicenter of the U.S. outbreak and represented the largest concentration of monkeypox individuals within a small geographical region (9). Subjects were screened and only those with close contact with monkeypox patients or infected prairie dogs were included in the study. Subjects who claimed to have had overt monkeypox disease symptoms provided us with authorization to confirm this with their primary physician. Subjects were categorized as suspect, probable, or confirmed cases of monkeypox based on the diagnostic and epidemiological criteria set forth by the CDC (28). Other control subjects consisted of Oregon residents (n=21) who had been recently vaccinated against smallpox or who had provided multiple serum samples at 33-37 years post-vaccination. Each subject provided informed written consent before signing HIPAA-compliant research authorization forms, filling out a medical history questionnaire, and providing a 50-100 mL blood sample that was processed at OHSU. Peripheral blood mononuclear cells (PBMC) were cryopreserved in aliquots and stored in liquid nitrogen. Plasma and serum samples were stored at −20° C. or −80° C. All clinical studies were approved by the Institutional Review Board of Oregon Health & Science University.

Intracellular cytokine staining (ICCS). Intracellular cytokine staining was performed as previously described (14). Briefly, PBMC were cultured at 37° C. with 6% CO₂ in RPMI containing 20 mM HEPES, L-glutamine, antibiotics, and 5% heat-inactivated FBS (Hyclone), with or without vaccinia virus (sucrose gradient-purified intracellular mature virus (IMV), vaccinia strain Western Reserve) at an MOI of 0.1. After 12 hours of culture, Brefeldin A (ICN) was added at a final concentration of 2 μg/mL for an additional 6 hours. The cells were stained overnight at 4° C. with antibodies specific for CD8β (clone 2ST8.5H7, Beckman Coulter) and CD4 (clone L200, PharMingen). Cells were fixed, permeabilized and stained intracellularly using antibodies to IFNγ (clone 4S.B3) and TNFα (clone Mab11), both from PharMingen. Samples were acquired on an LSRII instrument (Beckton Dickinson) using FACSDiva software (Beckton Dickinson), acquiring 1-2 million events per sample. Data was analyzed using FlowJo™ software and a live cell gate was performed using forward and side scatter characteristics. The number of TFNγ⁺TNFα⁺ T cells was quantitated after first gating on live CD4⁺CD8⁻ or CD4⁻CD8⁺ cells and subtracting the number of IFNγ⁺TNFα⁺ events from uninfected cultures. Each assay contained PBMC from a positive control (˜1 year post-smallpox vaccination), which scored 775±188 IFNγ⁺TNFα⁺CD4⁺T cells per 10⁶ CD4⁺ T cells and 1,844±585 IFNγ⁺TNFα⁺CD8⁺T cells per 10⁶ CD8⁺ T cells, respectively. One or more negative controls consisting of PBMC from vaccinia-naïve subjects were included in each assay.

ELISA. Vaccinia-specific and monkeypox-specific ELISA assays were performed as described herein (see also 14) using vaccinia (strain: WR) or monkeypox (strain: Zaire) whole virus lysate (inactivated by pretreatment with 3% H₂0₂ for >2 hours). An internal positive control was included on each plate to normalize ELISA values between plates and between assays performed on different days. Antibody titers were determined by log-log transformation of the linear portion of the curve, with 0.1 optical density (O.D.) units used as the endpoint and conversion performed on final values. Note: the same positive control sample (˜1 year post-smallpox vaccination) was used on both vaccinia and monkeypox-coated plates and normalized to the same ELISA value (e.g. normalized to 10,000 EU for each type of ELISA).

Peptide-specific ELISA assays were performed by coating 96-well flat-bottomed plates with a different 20-mer peptide (2 μg/mL in PBS) in each well. A number of candidate peptides were identified based on the monkeypox genome (accessed via the Poxvirus Bioinformatics Resource Center: http://www.poxvirus.org/). Peptides were purchased from Mimotope as 20 mers with 10 amino acid overlap. Each peptide (˜2 mg) was dissolved in 200 μL DMSO (Sigma, ACS spectrophotometric grade) followed by the addition of 200 μL of water (HPLC grade) for a final master stock concentration of ˜5 mg/mL. Inactivated vaccinia lysate was added to one well (functioning as a positive control for vaccinia-immune or monkeypox-immune samples and as a negative control for orthopoxvirus-naïve samples) and human plasma (containing IgG) was used to coat one well on each plate as an additional positive control. A single dilution (1:50) of plasma or serum was added to preblocked plates and incubated for 1 hour. After washing, plates were incubated for 1 hour with horseradish peroxidase-conjugated polyclonal goat antibodies to human IgG(γ) (Jackson ImmunoResearch Laboratories, Inc.). After an additional washing step, detection reagents were added, followed by 1M HCL, and the plates were read on an ELISA plate reader. Samples were considered positive for a particular peptide if they scored ≧2-fold over background on at least 2 to 3 different ELISA plates.

### Results:

A common misconception of the U.S. monkeypox outbreak is that infection required direct contact or direct inoculation through scratches/bites in order for monkeypox to be transmitted from infected prairie dogs to humans. Upon interviewing monkeypox patients in Wisconsin, applicants uncovered several cases in which monkeypox was transmitted to humans by indirect contact, possibly in the form of fomites or aerosol exposure (TABLE 3).

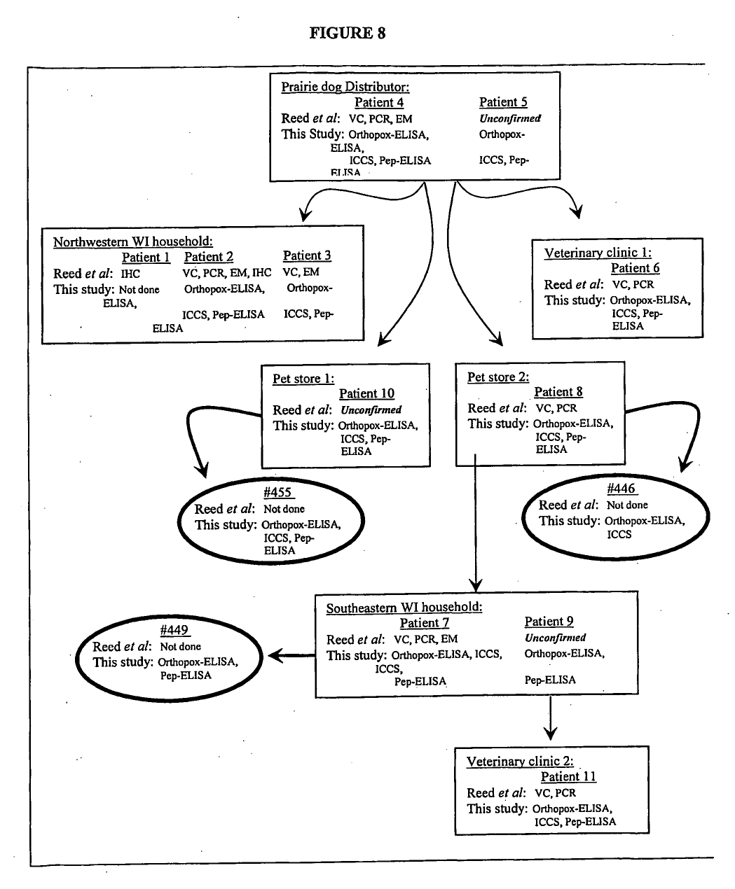

With respect to TABLE 3, subjects were asked to fill out a medical history questionnaire in which they described their smallpox vaccination status, disease symptoms, and location in which their exposure to monkeypox likely occurred. The 12 subjects in the upper portion of the table had not received smallpox vaccination whereas the 8 subjects in the lower portion of the table had received smallpox vaccination (typically confirmed by identification of smallpox vaccination scar on the left arm) and the estimated number of years after smallpox vaccination is listed (NA, not applicable). Questions regarding the symptoms of monkeypox infection were based on clinical diagnostic criteria set forth by the CDC during the outbreak²⁸ and consisted of rash (macular, papular, vesicular, or pustular; generalized or localized; discrete or confluent), fever (subjective or measured temperature≧37.4° C.) or other common symptoms including; headache, backache, swollen lymph nodes (lymphadenopathy), sore throat, cough, and shortness of breath. Subject #453 reported no rash, but exhibited all other monkeypox disease symptoms, some of which were moderate to severe. Subject #500 reported only one monkeypox lesion at the puncture site after being bitten by an infected prairie dog. Subjects #446, #449, and #455 reported no symptoms. Each subject reported exposure to monkeypox-infected prairie dogs in their homes or their place of work (see FIG. 8 and references (9, 11, 28-30) for further description of the locations of monkeypox exposure). Location of exposure abbreviations: PS2; Pet store 2, Dist; prairie dog distributor, VC2; Veterinary Clinic 2, SEHH; Southeastern household, NWHH; Northwestern household, PS1; Pet store 1, VC3; Veterinary Clinic 3 is not shown in FIG. 8 but is located in NW Wisconsin and was a location in which an ill prairie dog from the NWHH was treated. Putative route of exposure abbreviations: Direct C; Exposure by direct contact or handling of an infected prairie dog, Indirect C/A: Exposure by indirect contact with infected prairie dog or by possible aerosol exposure. In these cases, the subjects entered rooms in which infected prairie dogs were present, or had been present, but did not have direct contact with the infected animals.

In one case, a subject contracted monkeypox after an infected prairie dog was carried into her home when she was not present. The animal was apparently not placed on the floor or furniture and yet this subject, who had no other contact with monkeypox patients or prairie dogs, still contracted the disease. Cases of monkeypox in subjects who had not directly handled infected prairie dogs also occurred at other sites including a veterinary clinic in which a number of subjects contracted the disease by being present in (or later entering) a room in which an infected prairie dog was nebulized. Although applicants found no evidence of direct human-to-human spread in our limited assessment of this monkeypox outbreak, these results imply a word of caution to clinicians and other health care workers who might encounter virulent orthopoxviruses.

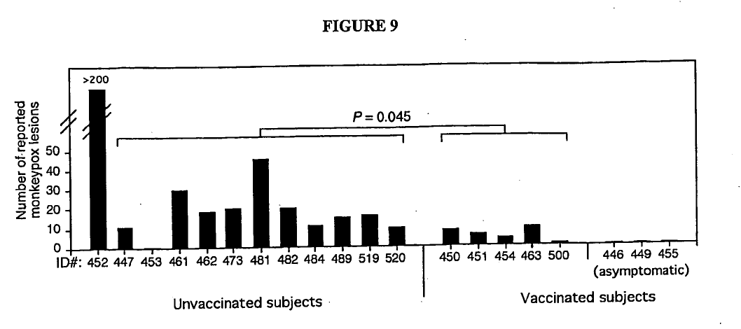

TABLE 3 shows the reported symptoms, vaccination status, and putative route of exposure for 12 cases of monkeypox in unvaccinated individuals, 5 cases of monkeypox in subjects who had previously received smallpox vaccination, and 3 cases of clinically inapparent monkeypox in previously vaccinated individuals. A comparison of the number of monkeypox lesions reported by vaccinated and unvaccinated subjects can be found in FIG. 9.

FIG. 9 shows a comparison of the number of monkeypox lesions reported by unvaccinated and vaccinated monkeypox patients. Subjects were asked to fill out a medical history questionnaire describing their history of monkeypox infection including the number of monkeypox lesions or “pocks” that developed during the course of this acute viral infection.

Based on retrospective self-reporting, it was unclear if the overall extent of other disease symptoms were modified in subjects who had been previously vaccinated. In our view, quantitation of the number of monkeypox lesions represented the least subjective symptom described in the medical history questionnaire. Based on the results from all monkeypox-infected subjects, there was an average of 33 monkeypox lesions in the unvaccinated group versus an average of 3.6 lesions in the vaccinated group, representing nearly a 10-fold difference overall. If we exclude the most severe case of monkeypox from the unvaccinated cohort (a patient with >200 monkeypox lesions and severe symptoms requiring hospitalization) and exclude the three clinically asymptomatic cases of monkeypox from the previously vaccinated cohort, then there were still significantly more monkeypox lesions observed in the unvaccinated subjects than in the vaccinated cohort (average=18 vs. 5.6 monkeypox lesions, respectively) (P=0.045, ANOVA).

This result is suggestive, but not necessarily correlative, of partial protection due to pre-existing immunity and is consistent with anecdotal evidence from another report indicating that vaccinated individuals may have a milder course of disease following monkeypox infection due to pre-existing immunity from childhood smallpox vaccination. In this case report of one family (Sejvar, J. J. et al. Human monkeypox infection: a family cluster in the midwestern United States J Infect Dis 190, 1833-40 (2004)), an unvaccinated mother exhibited ˜200 monkeypox lesions and her unvaccinated 6-year-old daughter exhibited ˜90 monkeypox lesions (in addition to severe encephalitis resulting in a coma that lasted for 12 days), whereas the previously vaccinated father developed just 2 monkeypox lesions and experienced only mild, flu-like symptoms for ˜48 hours.

In addition to the subjects in TABLE 3, blood samples were also obtained from 24 other subclinical contacts in Wisconsin (referred to as naïve contacts and vaccinia-immune contacts) as well as from vaccinia-naive and vaccinia-immune subjects who reside in Oregon.

Current tests used to help diagnose/confirm monkeypox infection are based solely on virus identification; this is a method of limited utility after the acute infection has cleared. Thus, as seen in the U.S. monkeypox outbreak in 2003 wherein about half of the reported cases of monkeypox remain unconfirmed (9), there is a need for reliable tests that can retrospectively define the scope of an outbreak.

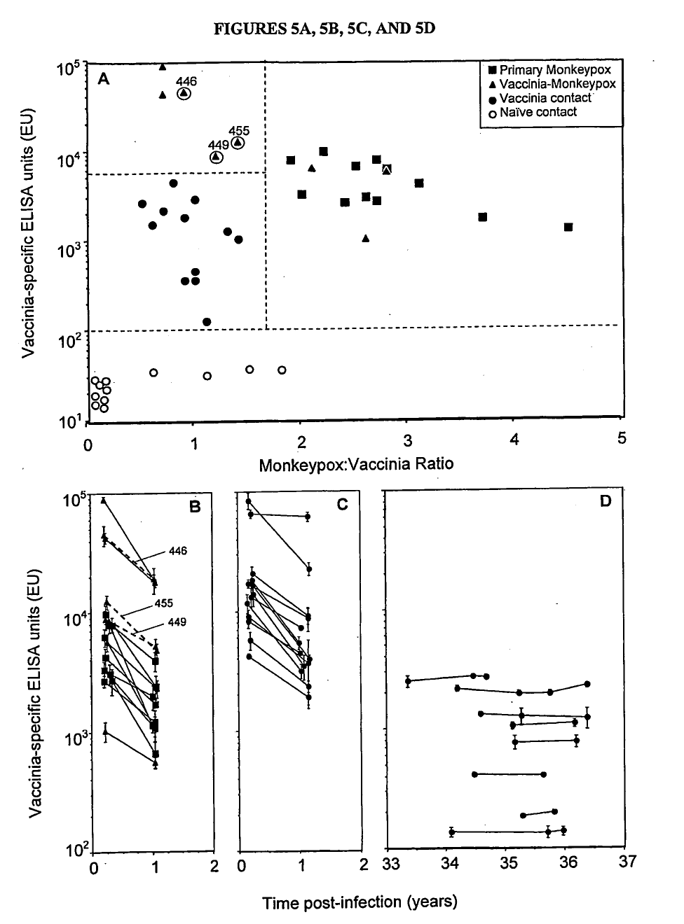

Applicants took an immunological approach to monkeypox diagnostics by initially performing ELISA assays using inactivated whole-virus lysates as a first attempt at discriminating monkeypox patients from uninfected contacts (FIGS. 5A-5D)).

FIGS. 5A-5D show antiviral antibody responses following orthopoxvirus infection. (A) Serum samples were drawn between 2 months to 1 year post-infection/exposure and tested on ELISA plates coated with equivalent amounts of inactivated vaccinia or monkeypox viral antigen. The monkeypox:vaccinia antibody ratio was determined by dividing the monkeypox-specific titers by the vaccinia-specific antibody titers (e.g. 2,000 EU against vaccinia and 4,000 EU against monkeypox results in a ratio of 2.0). A ratio could not be accurately determined on samples that scored <30 EU against vaccinia since these scores were below the limit of detection by this assay. (B) Antiviral antibody titers declined rapidly (mean: 62% decline, range: 35-85%) after recent monkeypox infection, even in the three subjects with clinically inapparent infections (dashed lines). (C) Similar results were observed in control subjects examined at similar defined time points following recent smallpox revaccination involving a live vaccinia virus infection (mean: 57% decline, range 7-76%). (D) In contrast, long-term antiviral antibody responses remained largely unaltered (mean: <0.5% annual decline, range: 0-3%), indicating that rapid antibody decline only occurs during the early stages after a recent orthopoxvirus infection. Each ELISA was repeated 2-6 times and symbols represent mean titers with error bars representing standard deviation. The numbers, 446, 449, and 455, represent the subject ID numbers of individuals with clinically inapparent monkeypox infections.

TABLE 3
Summary of prior vaccination status, symptoms, and exposure to monkeypox.

| | Years after | | | | | Swollen | | | | Location | Putative |
|---|---|---|---|---|---|---|---|---|---|---|---|
| | smallpox | | | | | lymph | Sore | | Shortness | of | route of |
| ID# | vaccination | Rash | Fever | Headache | Backache | nodes | throat | Cough | of breath | exposure | exposure |
| 447 | NA | X | X | X | X | | X | X | X | PS2 | Bite |
| 452 | NA | X | X | X | | X | X | X | | Dist | Bite/Scratch |
| 453 | NA | | X | X | X | X | X | X | X | Dist | Direct C |
| 461 | NA | X | X | X | | X | X | X | X | VC2 | Indirect C/A |
| 462 | NA | X | X | X | X | X | X | X | X | VC2 | Direct C |
| 473 | NA | X | X | X | | | X | X | X | VC2 | Scratch |
| 481 | NA | X | X | X | X | X | X | | X | VC2 | Direct C |
| 482 | NA | X | X | X | X | X | X | | | VC2 | Indirect C/A |
| 484 | NA | X | X | X | | X | X | X | X | VC2 | Direct C |
| 489 | NA | X | X | X | | X | X | | | SEHH | Indirect C/A |
| 519 | NA | X | X | | | X | X | | | NWHH | Existing |
| | | | | | | | | | | | scratch |
| 520 | NA | X | X | | | X | X | | | NWHH | Direct C |
| 450 | 34 | X | | X | X | X | X | X | | SEHH | Scratch |
| 451 | 32 | X | X | X | X | X | X | X | X | SEHH | Direct C |
| 454 | 38 | X | X | X | X | X | X | X | X | PS1 | Direct C |
| 463 | 38 | X | | X | | X | | | | VC1 | Existing cut |
| 500 | 34 | X | X | X | | X | | | | VC3 | Bite |
| 446 | 38 | | | | | | | | | PS2 | Indirect C/A |
| 449 | 13 | | | | | | | | | SEHH | Direct C |
| 455 | 29 | | | | | | | | | PS1 | Direct C |

Vaccinia ELISA assays exhibit 98% sensitivity and 100% specificity for detecting antiviral immunity, with antibody titers of unvaccinated individuals residing below 100 ELISA units (EU) (14). Diagnosing monkeypox in subjects under the age of 35 (i.e. born after routine smallpox vaccination was abandoned) was straightforward because unvaccinated contacts exhibited negligible antibody titers (<100 EU against vaccinia or monkeypox, n=12). In contrast, 12/12 of monkeypox patients demonstrated antibody titers ranging from 1,279-9,765 EU against vaccinia and 5,815-21,147 EU against monkeypox. Notably, these subjects exhibited high antibody titers that typically scored 2- to 4-fold higher against monkeypox than vaccinia. Although there is substantial cross-reactivity between orthopoxviruses, this indicates that additional antibody epitopes exist in monkeypox. This is consistent with earlier studies in which monkeypox-specific antibodies were still detected after cross-adsorption to vaccinia antigens (15-17).

Serological analysis of individuals over age 35 is more challenging to interpret because >90% of Americans over this age have been immunized and maintain lifelong vaccinia-specific antibody responses (18). Vaccinated contacts had antibody titers ranging between 123-4,408 EU against vaccinia and approximately a 1:1 ratio when comparing antibody titers against vaccinia versus monkeypox (FIG. 5A). In contrast, monkeypox infection of vaccinated subjects resulted in more heterogeneity. These subjects typically demonstrated high antibody titers against vaccinia and/or strong antibody titers to monkeypox, resulting in a high monkeypox:vaccinia ratio. In particular, three previously vaccinated subjects who had experienced no clinical symptoms of monkeypox (#446, #449, and #455 at 43, 13, and 29 years post-vaccination, respectively), demonstrated exceptionally high serological responses that were indicative of recent orthopoxvirus infection and could be clearly distinguished from uninfected vaccinia-immune contacts. This differential monkeypox:vaccinia ELISA approach provided the first indication that applicants had identified subjects with fully protective immunity against monkeypox.

If these serological results are indicative of recent monkeypox infection, then antibody titers should drop sharply after viral clearance. For this analysis, applicants compared antiviral antibody responses at early (2-4 months) and late (1 year) time points after monkeypox infection and found that antibody titers declined by ˜60%. This is strikingly similar to antibody decline following booster smallpox vaccination which is included here as a positive control (FIG. 5C). In contrast, long-term immunity following childhood vaccination is stable and averaged <1% decline over a 1-2 year period (FIG. 5D). Using >100 EU and >30% decline in antibody between paired acute and convalescent serum as diagnostic criteria indicative of recent orthopoxvirus infection, applicants achieved 100% (20/20) sensitivity for monkeypox detection, 92% (12/13) sensitivity for recent vaccinia infection/vaccination, and 100% specificity (0/8 long-term vaccinia-immune serum samples declined by 30% and 0/12 naïve contacts exhibit virus-specific antibodies >100 EU, FIG. 5A). These results provided compelling evidence that the three individuals with asymptomatic infections were indeed infected with monkeypox since they had antibody titers that declined rapidly, an expected result following recovery from a recent orthopoxvirus infection.

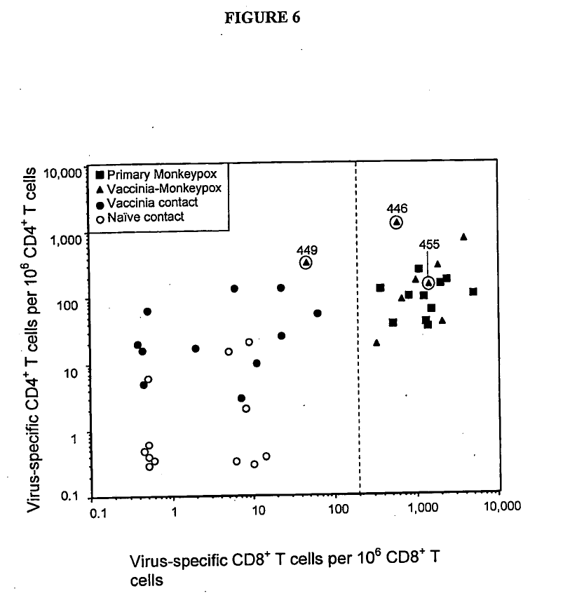

Intracellular cytokine staining analysis (ICCS) was used in monkeypox diagnosis by quantitating orthopoxvirus-specific CD4⁺ and CD8⁺ T cell responses (FIG. 6). Using a diagnostic cut-off of 200 IFNγ⁺TNFα⁺CD8⁺ T cells/10⁶ CD8⁺ T cells, applicants achieved 95% sensitivity (19/20 monkeypox-infected subjects scored >200) and 100% specificity (0/12 naïve contacts and 0/12 vaccinia-immune contacts scored >200).

Specifically, FIG. 6 shows diagnosis of recent monkeypox infection by quantitation of orthopoxvirus-specific T cells. The frequency of virus-specific T cells capable of producing both IFNγ and TNFα after direct ex vivo stimulation with vaccinia virus was determined by intracellular cytokine staining (ICCS). Samples that scored below detection were graphed with values of <1 per 10⁶. The vertical dashed line represents the diagnostic cut-off of virus-specific CD8⁺ T cells used for distinguishing recently infected monkeypox patients from uninfected naïve contacts and vaccinia-immune contacts (immunized >20 years previously). This data and comparison with previous studies with a large number of vaccinia-immune subjects¹⁴, indicates that this approach provides ≧95% sensitivity and >97% specificity for detecting a recent orthopoxvirus infection.

In previous work (14), <4% (0/26) of vaccinia-naïve subjects and <3% (7/256) of vaccinia-immune subjects at >20 years post-vaccination exhibited antiviral responses of ≧200 IFNγ⁺TNFα⁺CD8⁺ T cells/10⁶ CD8⁺ T cells—indicating this diagnostic cut-off has 100% specificity in vaccinia-naïve subjects and >97% specificity overall. Two of three asymptomatic individuals (#446 and #455) showed high T cell levels comparable to subjects with clinical symptoms, again verifying their recent monkeypox infection. Only one monkeypox-infected subject (#449) did not clearly segregate with other monkeypox patients, but this subject's virus-specific T cell responses were still proportionally high and, if similar to acute vaccinia infection (19), may have been higher if a blood sample could have been obtained earlier than 3 months post-exposure. This is the first demonstration of a T cell-based diagnostic approach capable of distinguishing 100% of clinically-apparent monkeypox-infected individuals from naïve and vaccinia-immune contacts and provides independent confirmation of applicants' initial serological results.

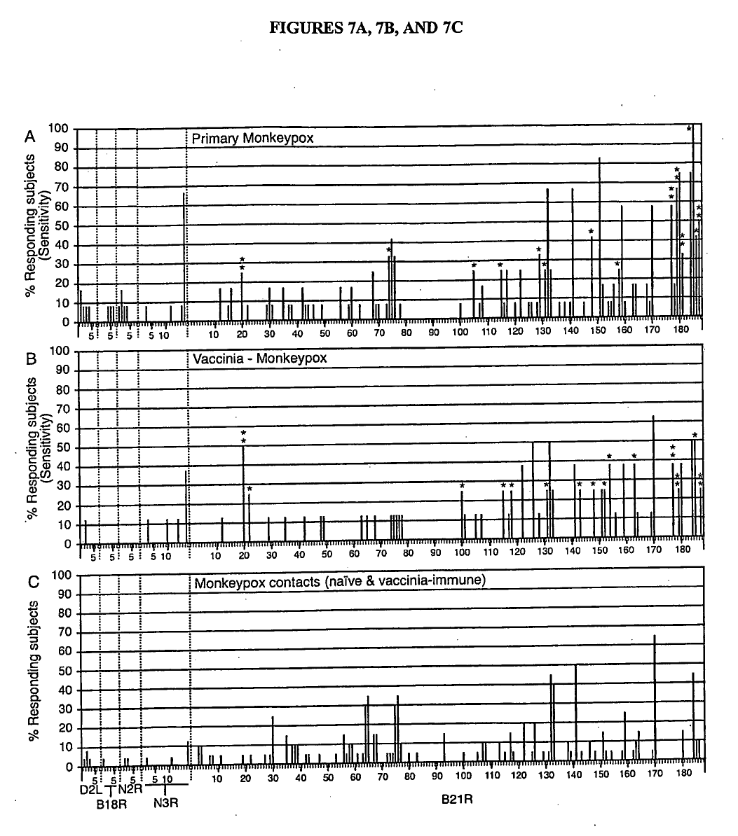

Many monkeypox-infected patients exhibited higher antibody responses against monkeypox than against vaccinia, suggesting the existence of novel epitopes (FIG. 5). To identify potential epitopes, candidate genes were identified in monkeypox (20) that are not present in the vaccinia genome, including D2L, B18R, N2R, N3R, and B21R and overlapping peptides were used for screening linear antibody epitopes (FIG. 7A-7C).

Specifically FIG. 7 shows analysis of monkeypox-specific peptide ELISA assays for diagnosing monkeypox infection. The numbers on the X axis are the exemplary peptide numbers (peptide #1 is amino acids 1-20, peptide #2 represents amino acids 10-30, peptide #3 represents amino acids 20-40, etc.) and each peptide is 20 amino acids long and overlaps the previous peptide by 10 amino acids. Exemplary peptide #67, for example, represents B21R amino acids 660-680. Serum or plasma samples (1:50 dilution) obtained at 2 months to 1 year post-infection/exposure were incubated on ELISA plates coated with an individual peptide in each well. Samples were scored positive for a particular peptide if they scored ≧2-fold over background on at least 2 to 3 different ELISA plates. Panels A-C show the percentage of samples that scored positive against peptides from putative monkeypox proteins, D2L, B18R, N2R, N3R, and B21R. A, Primary monkeypox (n=12), B, Vaccinia-monkeypox (n=8), C, Monkeypox contacts, vaccinia-naive and vaccinia-immune (n=20 to 24). The major immunodominant peptide epitopes are marked with one asterisk (*) for those with ≧90% specificity or with two asterisks for peptide epitopes with 100% specificity. Monkeypox contacts were evenly divided between vaccinia-naïve and vaccinia-immune subjects.

Of the smaller putative gene products (D2L, B18R, N2R, and N3R), the carboxy-terminus of the N3R gene was modestly promising with 67% sensitivity for identifying unvaccinated monkeypox cases, 38% sensitivity for vaccinated monkeypox cases, and 88% specificity among uninfected naïve or vaccinia-immune contacts. The larger B21R gene product (1,879 amino acids) was highly immunogenic, with 100% of monkeypox-infected subjects responding to ≧3 epitopes and 60% of these subjects responding to ≧10 peptides (range: 3-41 peptides). The most immunogenic B21R epitope was peptide #185, which elicited 100% (12/12) sensitivity in unvaccinated monkeypox patients, 50% (4/8) sensitivity in vaccinated monkeypox patients, and 90% specificity (FIG. 3). Of the asymptomatic individuals unknowingly infected with monkeypox, Subject #446 had the highest overall antibody response (FIG. 5A) and the strongest CD4⁺ T cell response (FIG. 2), but responded to only four B21R epitopes. However, this individual responded to B21R peptides #126 and #180, both of which exhibit reasonably high specificity (80% and 85%, respectively). Subject #455 responded to five B21R epitopes including peptide #20 and #148 (100% and 95% specificity, respectively) and Subject #449 responded to nine B21R epitopes including peptide #20 and #115 (100% and 95% specificity, respectively). Together, these results indicate that linear peptides provide an effective and sensitive approach to monkeypox diagnostics.

There were 39 reported cases of monkeypox in Wisconsin; 18 laboratory-confirmed, and 11 described by Reed et al. (11). Of these subjects, we found 100% concordance with the diagnostic results obtained from monkeypox patients with clear clinical disease symptoms and who were positive by virological assays such as electron microscopy (EM), viral culture (VC), immunohistochemistry (IHC) and/or PCR (n=7). In addition, applicants' approach confirmed three probable/suspect cases of monkeypox described in this earlier study (11) who had exposure to monkeypox, demonstrated most or all clinical symptoms of monkeypox, but who were negative by virological analysis (FIG. 8). The relationship between the three previously unidentified individuals who experienced asymptomatic monkeypox infections can now be placed into the context of the greater monkeypox outbreak.

FIG. 8 shows the relationship between reported and unreported (i.e. asymptomatic) monkeypox infections. This figure was modified from a similar flow-chart diagram published by Reed et al. (11) and shows the relationship between different monkeypox survivors in the context of the WI monkeypox outbreak. Patients 4 and 5 are subjects who purchased 39 prairie dogs from an Illinois distributor and sold 2 prairie dogs to the family in the Northwestern WI household, the site of the first recorded case of human monkeypox in the United States. Two prairie dogs were sold to Pet store 1, 10 prairie dogs were sold to Pet store 2, and an ill prairie dog was treated at Veterinary Clinic 1. Pet store 2 then sold a prairie dog to subjects in a Southeastern WI household and when the animal showed disease symptoms, it was treated at Veterinary Clinic 2. Further details of the outbreak are published elsewhere^{9,11,28-30}. The diagnostic methodology used by Reed et al. and the new immunological techniques developed here are provided for comparison. The previous diagnostic criteria involved virological techniques including electron microscopy (EM), viral culture (VC), immunohistochemistry (IHC), and polymerase-chain-reaction (PCR). Our study used diagnostic procedures including vaccinia whole-virus ELISA with a positive titer (i.e. >100 EU) followed by >30% decline in antibody titers between paired acute and convalescent serum as diagnostic criteria indicative of a recent orthopoxvirus infection (Orthopox-ELISA). Intracellular cytokine staining (ICCS) was used to quantitate orthopoxvirus-specific T cells, with >200 antiviral CD8⁺ T cells/10⁶ as diagnostic criteria indicative of monkeypox infection. Monkeypox B21R peptide ELISA (Pep-ELISA) results were considered positive for monkeypox infection if responses were observed against one or more B21R peptides that have ≧90% specificity and ≧90% sensitivity. Samples were labeled as Unconfirmed if they were previously listed as probable or suspect cases according to CDC criteria²⁸. In these cases, the virological diagnostic methods scored negative or equivocal even though the subjects had known contact with monkeypox-infected prairie dogs and experienced many or all of the characteristic disease symptoms of monkeypox infection. The previously reported cases of clinically apparent monkeypox are shown in rectangles and the three asymptomatic monkeypox cases identified in this current study are shown in ovals.

In summary of this EXAMPLE V, applicants used a multi-faceted approach to diagnosing clinically apparent and inapparent monkeypox infections. Using an optimized differential vaccinia:monkeypox whole-virus ELISA test, applicants identified subjects with recent monkeypox infection and these results were confirmed by quantitation of antibody decay rates during the first year following recovery. A cell-mediated diagnostic approach was also developed wherein applicants measured the number of orthopoxvirus-specific T cells by ICCS. This proved to be an effective, independent approach capable of correctly diagnosing all clinically apparent monkeypox patients and 2/3 inapparent monkeypox infections. Serological studies using overlapping peptides revealed the monkeypox B21R protein as an important antibody target with several key immunodominant epitopes. These peptide reagents worked well in retrospective serological analysis of a monkeypox outbreak and have utility for the development of monkeypox-specific monoclonal antibodies suitable for rapid, direct detection of the virus. Similar technology could potentially be developed to detect and/or monitor a deliberate smallpox attack.

Applicants' diagnostic approaches confirmed monkeypox infection in patients who were previously listed as probable or suspect (FIG. 8). These patients demonstrated multiple disease symptoms indicative of monkeypox, but tested negative or equivocal by current virological techniques. In some cases, this may have been due to subjects not immediately seeking medical attention, and after the infection had resolved, virological assays such as PCR would no longer be capable of making a positive diagnosis. Although orthopoxvirus-specific PCR can detect as few as 25 genome equivalents in the laboratory (21), it only detected monkeypox in 6/11 cases (55% sensitivity) of clinically overt monkeypox (11). An advantage of using the immunological assays described here is that a positive diagnosis can be made retrospectively due to persisting immunity. Monkeypox continues to be a problem in Africa, with outbreaks that are difficult to monitor due to inconsistencies in epidemiological methodology and the limitations of current diagnostics (13). Antiviral antibody and T cell responses begin to rise at or near the time of disease onset, so novel and highly sensitive immunological techniques may potentially prove effective for monkeypox diagnosis during an ongoing outbreak, but further studies are necessary to determine the earliest time in which monkeypox infection can be reliably detected by these methods.

If this study was performed in Europe where cowpox is endemic (22-24), or in Africa where monkeypox outbreaks occur (2-6), then one could not rule out the possibility that long-term immunity was due to intermittent re-exposure to crossreactive orthopoxviruses. This is unlikely to be an issue in the instant study because the last case of smallpox in the U.S. was in 1949, routine smallpox vaccination was discontinued in 1972, and there are no orthopoxviruses indigenous to North America that are known to infect humans. Thus, the U.S. monkeypox outbreak provided a rare opportunity to measure protective antiviral immunity in the absence of endemic orthopoxviruses.

Greater than 100 million Americans have received smallpox vaccination and a question arising from this report is; how might protective immunity against monkeypox relate to protection against smallpox? Monkeypox serves as an informative surrogate for smallpox in that it is a human pathogen capable of inducing lethal infections in 4-25% of those afflicted (2-6) and smallpox vaccination is cross-protective (3). Applicants examined the immune responses and clinical outcome of subjects infected with a West African strain of monkeypox, which may or may not exhibit the same mortality rates observed in previous monkeypox outbreaks. There are many factors that play a role in monkeypox-induced mortality including the strain of virus involved, the route of infection, the age, nutritional status, immune status and vaccination status of the host, and access to sophisticated medicinal care (13). In our study, we identified 5 vaccinated subjects who contracted monkeypox and 3 vaccinated subjects who demonstrated full protection against the onset of monkeypox-induced disease. The results broadly indicate that almost half of vaccinated individuals (3/8) maintain long-term protective immunity against monkeypox. Interestingly, previous analysis of vaccinia-specific antibody levels in >300 vaccinees showed that ˜50% have neutralizing antibody titers of ≧1:32 (14) which prior reports suggested would provide fully protective immunity against smallpox (25, 26). In an elegant study involving >300 subjects, the overt smallpox attack rate was 68.8% among unvaccinated contacts compared to only 3.2% in vaccinated contacts (27). Remarkably, 55% (78/142) of vaccinated household contacts had clinically inapparent smallpox infections, indicative of pre-existing, fully protective immunity (27). A main point of this current study is that applicants' findings are consistent with this much larger previous report (27), and demonstrates that some level of protective immunity is likely to exist in contemporary subjects who have received smallpox vaccination in the distant past.

### REFERENCES USED IN THIS EXAMPLE V

1. Henderson, D. A. The looming threat of bioterrorism. Science 283, 1279-82. (1999).
2. Jezek, Z. et al. Human monkeypox: a study of 2,510 contacts of 214 patients. J Infect Dis 154, 551-5 (1986).
3. Jezek, Z., Szczeniowski, M., Paluku, K. M. & Mutombo, M. Human monkeypox: clinical features of 282 patients. J Infect Dis 156, 293-8 (1987).
4. Jezek, Z., Grab, B., Paluku, K. M. & Szczeniowski, M. V. Human monkeypox: disease pattern, incidence and attack rates in a rural area of northern Zaire. Trop Geogr Med 40, 73-83 (1988).
5. Hutin, Y. J. et al. Outbreak of human monkeypox, Democratic Republic of Congo, 1996 to 1997. Emerg Infect Dis 7, 434-8 (2001).
6. Meyer, H. et al. Outbreaks of disease suspected of being due to human monkeypox virus infection in the democratic republic of congo in 2001. J Clin Microbiol 40, 2919-21 (2002).
7. Smith, G. L. & McFadden, G. Smallpox: anything to declare? Nat Rev Immunol 2, 521-7 (2002).
8. CDC. Update: multistate outbreak of monkeypox—Illinois, Indiana, Kansas, Missouri, Ohio, and Wisconsin, 2003. MMWR Morb Mortal Wkly Rep 52, 642-6 (2003).
9. Gross, E. Update on emerging infections: news from the Centers for Disease Control and prevention. Update: Multistate outbreak of monkeypox—Illinois, Indiana, Kansas, Missouri, Ohio, and Wisconsin, 2003. Ann Emerg Med 42, 660-2; discussion 662-4 (2003).
10. Seward, J. F. et al. Development and experience with an algorithm to evaluate suspected smallpox cases in the United States, 2002-2004. Clin Infect Dis 39, 1477-83 (2004).
11. Reed, K. D. et al. The detection of monkeypox in humans in the Western Hemisphere. N Engl J Med 350, 342-50 (2004).
12. Klein, K. R, Atas, J. G. & Collins, J. Testing emergency medical personnel response to patients with suspected infectious disease. Prehospital Disaster Med 19, 256-65 (2004).
13. Di Giulio, D. B. & Eckburg, P. B. Human monkeypox: an emerging zoonosis. Lancet Infect Dis 4, 15-25 (2004).
14. Hammarlund, E. et al. Duration of antiviral immunity after smallpox vaccination. Nature Medicine 9, 1131-1137 (2003).
15. Esposito, J. J., Obijeski, J. F. & Nakano, J. H. Serological relatedness of monkeypox, variola, and vaccinia viruses. J Med Virol 1, 35-47 (1977).
16. Hutchinson, H. D., Ziegler, D. W., Wells, D. E. & Nakano, J. H. Differentiation of variola, monkeypox, and vaccinia antisera by radioimmunoassay. Bull World Health Organ 55, 613-23 (1977).
17. Jezek, Z. et al. Serological survey for human monkeypox infections in a selected population in Zaire. J Trop Med Hyg 90, 31-8 (1987).
18. Slifka, M. K. Immunological memory to viral infection. Curr Opin Immunol 16, 443-50 (2004).
19. Amara, R. R., Nigam, P., Sharma, S., Liu, J. & Bostik, V. Long-lived poxvirus immunity, robust CD4 help, and better persistence of CD4 than CD8 T cells. J Virol 78, 3811-6 (2004).
20. Shchelkunov, S. N. et al. Analysis of the monkeypox virus genome. Virology 297, 172-94 (2002).
21. Sofi Ibrahim, M. et al. Real-time PCR assay to detect smallpox virus. J Clin Microbiol 41, 3835-9 (2003).
22. Baxby, D., Bennett, M. & Getty, B. Human cowpox 1969-93: a review based on 54 cases. Br J Dermatol 131, 598-607 (1994).
23. Hawranek, T. et al. Feline orthopoxvirus infection transmitted from cat to human. J Am Acad Dermatol 49, 513-8 (2003).
24. Pelkonen, P. M. et al. Cowpox with severe generalized eruption, Finland. Emerg Infect Dis 9, 1458-61 (2003).
25. Mack, T. M., Noble, J., Jr. & Thomas, D. B. A prospective study of serum antibody and protection against smallpox. Am J Trop Med Hyg 21, 214-8. (1972).
26. Sarkar, J. K., Mita, A. C. & Mukherjee, M. K. The minimum protective level of antibodies in smallpox. Bull World Health Organ 52, 307-11 (1975).
27. Heiner, G. G. et al. A study of inapparent infection in smallpox. Am J Epidemiol 94, 252-68. (1971).
28. CDC. Update: multistate outbreak of monkeypox—Illinois, Indiana, Kansas, Missouri, Ohio, and Wisconsin, 2003. MMWR Morb Mortal Wkly Rep 52, 561-4 (2003).
29. CDC. Multistate outbreak of monkeypox—Illinois, Indiana, and Wisconsin, 2003. MMWR Morb Mortal Wkly Rep 52, 537-40 (2003).
30. Anderson, M. G., Frenkel, L. D., Homann, S. & Guffey, J. A case of severe monkeypox virus disease in an American child: emerging infections and changing professional values. Pediatr Infect Dis J 22, 1093-6; discussion 1096-8 (2003).

### Example VI

### Monkeypox Antigens were Identified that are Diagnostic for Both Smallpox and Monkeypox, and Monkeypox Antigens were Identified that are Specific for Smallpox

In this EXAMPLE applicants have used the inventive SABRE platform to identify monkeypox antigens that are diagnostic for both smallpox and monkeypox, and to identify monkeypox antigens that are specific for smallpox.

TABLE 4 shows, according to particular aspects of the present invention, various exemplary monkeypox B21R peptides that are cross-reactive and recognized by smallpox survivors.

TABLE 4
Antibody cross-reactivity among Smallpox survivors

| Peptide | Specificity ≧ 90% | Specificity < 90% |
|---|---|---|
| *3 | X | |
| *4 | X | |
| *11 | X | |
| *19 | X | |
| *27 | X | |
| *29 | X | |
| *30 | | X |
| *32 | X | |
| *35 | | X |
| *37 | X | |
| 42 | X | |
| *44 | X | |
| *50 | X | |
| *60 | X | |
| *63 | X | |
| *64 | | X |
| *65 | | X |
| **67 | | X |
| *68 | | X |
| 73 | X | |
| *74 | x | |
| **75 | | X |
| *76 | | X |
| *78 | X | |
| *79 | X | |
| *80 | X | |
| 100 | X | |
| *115 | X | |
| *126 | | X |
| *129 | X | |
| *131 | X | |
| **132 | | X |
| **141 | | X |
| *151 | | X |
| **152 | X | |
| *155 | X | |
| **159 | X | |
| 166 | X | |
| **168 | X | |
| *169 | X | |
| **170 | | X |
| 172 | X | |
| *174 | X | |
| *177 | X | |
| **178 | X | |
| *180 | | X |
| **184 | | X |
| *185 | X | |
| *186 | X | |

Peptides were designed based on Monkeypox B21R protein sequence.
Specificity is based on 20 naive or vaccinia immune subjects.
*Smallpox international serum standard (pool of 63 subjects, approximately 1 month post-smallpox infection).
**Smallpox international serum standard AND smallpox survivors.

TABLE 5 shows, according to particular aspects of the present invention, various exemplary peptides that are diagnostic for both smallpox and monkeypox.

TABLE 5
Monkeypox B21R/Variola Major (Bangladesh) B22R alignment; 84.3% identity in
1914 residues overlap. Numbered boxes represent peptides (designed using Monkeypox B21R
sequence) for which both Monkeypox and Smallpox survivors have antibody specificity.

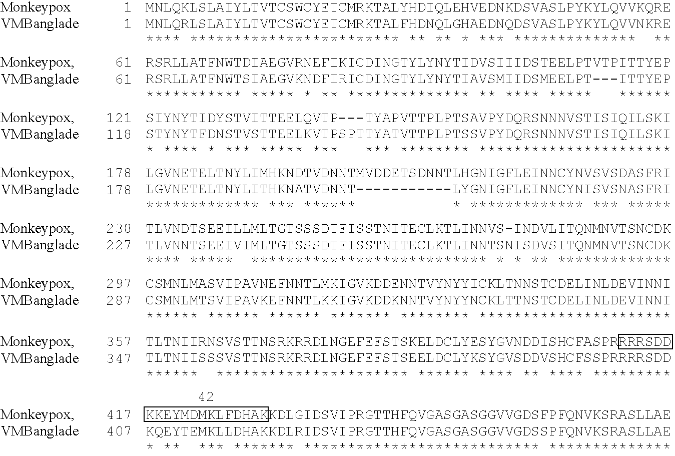

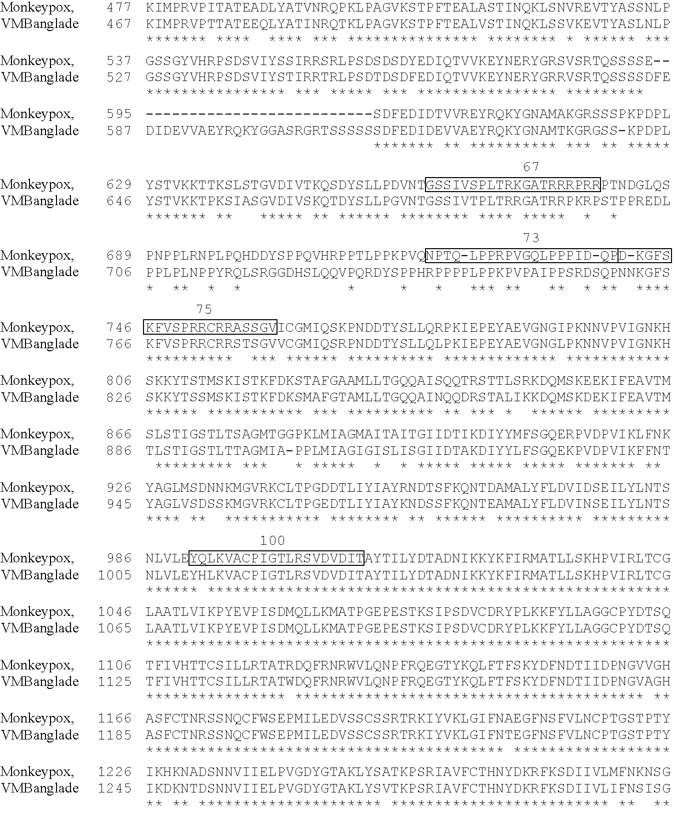

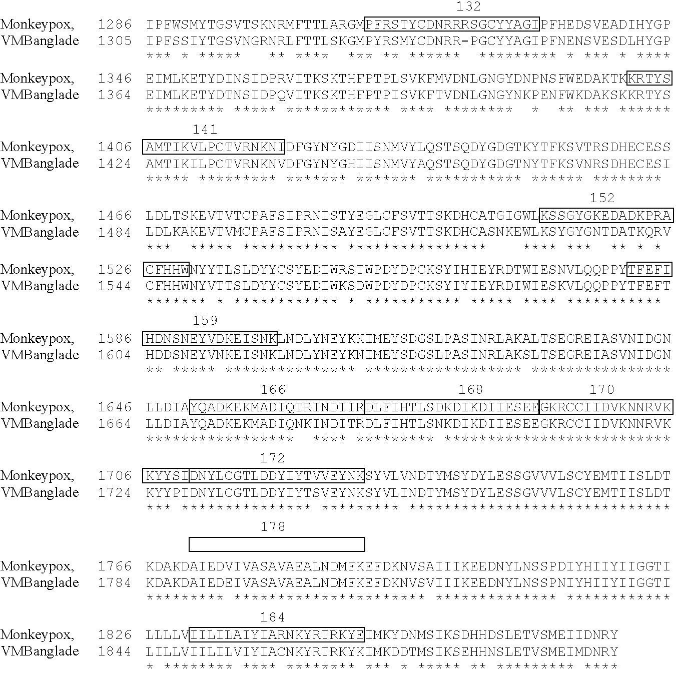

TABLE 6 lists multiple (187 different B21R peptides) exemplary monkeypox B21R peptides that are, according to particular aspects of the present invention, diagnostic for both smallpox and monkeypox. The stars indicate how “good” the peptides work by indicating specificity. 90% specificity means, for example, that 18/20 negative controls do not respond to the peptide, and 100% specificity means, for example, that none of the negative controls responded to the specific peptide.

TABLE 6
Exemplary peptides of monkeypox B21R protein

| | | SEQ | | | |
|---|---|---|---|---|---|
| | Peptides | ID NO. | | Hydro | MolWt |
| 1 | H-MNLQKLSLAIYLTVTCSWCY | 45 | —OH | 0.70* | 2,350.87 |
| 2 | H-YLTVTCSWCYETCMRKTALY | 46 | —OH | 0.57 | 2,435.91 |
| 3 | H-ETCMRKTALYHDIQLEHVED | 47 | —OH | 0.17 | 2,431.75 |
| 4 | H-HDIQLEHVEDNKDSVASLPY | 48 | —OH | 0.12 | 2,309.49 |
| 5 | H-NKDSVASLPYKYLQVVKQRE | 49 | —OH | 0.08 | 2,365.73 |
| 6 | H-KYLQVVKQRERSRLLATFNW | 50 | —OH | 0.21 | 2,536.00 |
| 7 | H-RSRLLATFNWTDIAEGVRNE | 51 | —OH | 0.16 | 2,348.62 |
| 8 | H-TDIAEGVRNEFIKICDINGT | 52 | —OH | 0.22 | 2,208.49 |
| 9 | H-FIKICDINGTYLYNYTIDVS | 53 | —OH | 0.51 | 2,355.71 |
| 10 | H-YLYNYTIDVSIIIDSTEELP | 54 | —OH | 0.49 | 2,361.64 |
| 11 | H-IIIDSTEELPTVTPITTYEP | 55 | —OH | 0.46 | 2,232.53 |
| 12 | H-TVTPITTYEPSIYNYTIDYS | 56 | —OH | 0.43 | 2,341.57 |
| 13 | H-SIYNYTIDYSTVITTEELQV | 57 | —OH | 0.40 | 2,352.59 |
| 14 | H-TVITTEELQVTPTYAPVTTP | 58 | —OH | 0.42 | 2,161.45 |
| 15 | H-TPTYAPVTTPLPTSAVPYDQ | 59 | —OH | 0.40 | 2,119.37 |
| 16 | H-LPTSAVPYDQRSNNNVSTIS | 60 | —OH | 0.16 | 2,163.34 |
| 17 | H-RSNNNVSTISIQILSKILGV | 61 | —OH | 0.33 | 2,156.53 |
| 18 | H-IQILSKILGVNETELTNYLI | 62 | —OH | 0.51 | 2,274.70 |
| 19 | H-NETELTNYLIMHKNDTVDNN | 63 | —OH | 0.04 | 2,378.57 |
| 20 | H-MHKNDTVDNNTMVDDETSDN | 64 | —OH | −0.18 | 2,295.37 |
| 21 | H-TMVDDETSDNNTLHGNIGFL | 65 | —OH | 0.17 | 2,193.35 |
| 22 | H-NTLHGNIGFLEINNCYNVSV | 66 | —OH | 0.39 | 2,221.49 |
| 23 | H-EINNCYNVSVSDASFRITLV | 67 | —OH | 0.35 | 2,244.53 |
| 24 | H-SDASFRITLVNDTSEEILLM | 68 | —OH | 0.34 | 2,254.56 |
| 25 | H-NDTSEEILLMLTGTSSSDTF | 69 | —OH | 0.26 | 2,161.34 |
| 26 | H-LTGTSSSDTFISSTNITECL | 70 | —OH | 0.35 | 2,077.26 |
| 27 | H-ISSTNITECLKTLINNVSIN | 71 | —OH | 0.37 | 2,177.52 |
| 28 | H-KTLINNVSINDVLITQNMNV | 72 | —OH | 0.34 | 2,243.63 |
| 29 | H-DVLITQNMNVTSNCDKCSMN | 73 | —OH | 0.23 | 2,230.56 |
| 30 | H-TSNCDKCSMNLMASVIPAVN | 74 | —OH | 0.35 | 2,098.49 |
| 31 | H-LMASVIPAVNEFNNTLMKIG | 75 | —OH | 0.46 | 2,162.62 |
| 32 | H-EFNNTLMKIGVKDDENNTVY | 76 | —OH | 0.07 | 2,344.60 |
| 33 | H-VKDDENNTVYNYYICKLTTN | 77 | —OH | 0.13 | 2,410.66 |
| 34 | H-NYYICKLTTNSTCDELINLD | 78 | —OH | 0.35 | 2,336.64 |
| 35 | H-STCDELINLDEVINNITLTN | 79 | —OH | 0.32 | 2,234.48 |
| 36 | H-EVINNITLTNIIRNSVSTTN | 80 | —OH | 0.27 | 2,216.49 |
| 37 | H-IIRNSVSTTNSRKRRDLNGE | 81 | —OH | −0.14 | 2,316.58 |
| 38 | H-SRKRRDLNGEFEFSTSKELD | 82 | —OH | −0.17 | 2,414.63 |
| 39 | H-FEFSTSKELDCLYESYGVND | 83 | —OH | 0.23 | 2,346.53 |
| 40 | H-CLYESYGVNDDISHCFASPR | 84 | —OH | 0.32 | 2,276.51 |
| 41 | H-DISHCFASPRRRRSDDKKEY | 85 | —OH | −0.16 | 2,466.74 |
| 42 | H-RRRSDDKKEYMDMYLFDHAK | 30 | —OH | −0.23 | 2,569.97 |
| 43 | H-MDMKLFDHAKKDLGIDSVIP | 86 | —OH | 0.26 | 2,273.72 |
| 44 | H-KDLGIDSVIPRGTTHFQVGA | 87 | —OH | 0.26 | 2,111.40 |
| 45 | H-RGTTHFQVGASGASGGVVGD | 88 | —OH | 0.13 | 1,859.99 |
| 46 | H-SGASGGVVGDSFPFQNVKSR | 89 | —OH | 0.13 | 1,996.18 |
| 47 | H-SFPFQNVKSRASLLAEKIMP | 90 | —OH | 0.32 | 2,263.71 |
| 48 | H-ASLLAEKIMPRVPITATEAD | 91 | —OH | 0.31 | 2,126.52 |
| 49 | H-RVPITATEADLYATVNRQPK | 92 | —OH | 0.14 | 2,243.56 |
| 50 | H-LYATVNRQPKLPAGVKSTPF | 93 | —OH | 0.29 | 2,187.59 |
| 51 | H-LPAGVKSTPFTEALASTINQ | 94 | —OH | 0.32 | 2,045.34 |
| 52 | H-TEALASTINQKLSNVREVTY | 95 | —OH | 0.16 | 2,237.51 |
| 53 | H-KLSNVREVTYASSNLPGSSG | 96 | —OH | 0.10 | 2,066.27 |
| 54 | H-ASSNLPGSSGYVHRPSDSVI | 97 | —OH | 0.20 | 2,030.20 |
| 55 | H-YVHRPSDSVIYSSIRRSRLP | 98 | —OH | 0.20 | 2,388.73 |
| 56 | H-YSSIRRSRLPSDSDSDYEDI | 99 | —OH | −0.04 | 2,361.48 |
| 57 | H-SDSDSDYEDIQTVVKEYNER | 100 | —OH | −0.15 | 2,392.44 |
| 58 | H-QTVVKEYNERYGRSVSRTQS | 101 | —OH | −0.10 | 2,387.61 |
| 59 | H-YGRSVSRTQSSSSESDFEDI | 102 | —OH | −0.05 | 2,237.29 |
| 60 | H-SSSESDFEDIDTVVREYRQK | 103 | —OH | −0.10 | 2,390.52 |
| 61 | H-DTVVREYRQKYGNAMAKGRS | 104 | —OH | −0.12 | 2,329.64 |
| 62 | H-YGNAMAKGRSSSPKPDPLYS | 105 | —OH | 0.06 | 2,126.39 |
| 63 | H-SSPKPDPLYSTVKKTTKSLS | 106 | —OH | 0.08 | 2,164.50 |
| 64 | H-TVKKTTKSLSTGVDIVTKQS | 107 | —OH | 0.07 | 2,121.47 |
| 65 | H-TGVDIVTKQSDYSLLPDVNT | 108 | —OH | 0.24 | 2,165.40 |
| 66 | H-DYSLLPDVNTGSSIVSPLTR | 109 | —OH | 0.33 | 2,134.39 |
| 67 | H-GSSIVSPLTRKGATRRRPRR | 31 | −OH | −0.09 | 2,251.64 |
| 68 | H-KGATRRRPRRPTNDGLQSPN | 110 | —OH | −0.24 | 2,277.55 |
| 69 | H-PTNDGLQSPNPPLRNPLPQH | 111 | —OH | 0.18 | 2,192.43 |
| 70 | H-PPLRNPLPQHDDYSPPQVHR | 112 | —OH | 0.17 | 2,363.64 |
| 71 | H-DDYSPPQVHRPPTLPPKPVQ | 113 | —OH | 0.22 | 2,268.57 |
| 72 | H-PPTLPPKPVQNPTQLPPRPV | 114 | —OH | 0.37 | 2,173.60 |
| 73 | H-NPTQLPPRPVGQLPPPIDQP | 32 | —OH | 0.35 | 2,161.50 |
| 74 | H-GQLPPPIDQPDKGFSKFVSP | 115 | —OH | 0.28 | 2,154.47 |
| 75 | H-DKGFSKFVSPRRCRRASSGV | 33 | —OH | −0.01 | 2,240.59 |
| 76 | H-RRCRRASSGVICGMIQSKPN | 116 | —OH | 0.11 | 2,219.66 |
| 77 | H-ICGMIQSKPNDDTYSLLQRP | 117 | —OH | 0.27 | 2,279.64 |
| 78 | H-DDTYSLLQRPKIEPEYAEVG | 118 | —OH | 0.13 | 2,323.56 |
| 79 | H-KIEPEYAEVGNGIPKNNVPV | 119 | —OH | 0.15 | 2,167.46 |
| 80 | H-NGIPKNNVPVIGNKHSKKYT | 120 | —OH | 0.03 | 2,208.56 |
| 81 | H-IGNKHSKKYTSTMSKISTKF | 121 | —OH | 0.05 | 2,286.70 |
| 82 | H-STMSKISTKFDKSTAFGAAM | 122 | —OH | 0.18 | 2,109.47 |
| 83 | H-DKSTAFGAAMLLTGQQAISQ | 123 | —OH | 0.26 | 2,038.32 |
| 84 | H-LLTGQQAISQQTRSTTLSRK | 124 | —OH | 0.11 | 2,217.53 |
| 85 | H-QTRSTTLSRKDQMSKEEKIF | 125 | —OH | −0.09 | 2,413.75 |
| 86 | H-DQMSKEEKIFEAVTMSLSTI | 126 | —OH | 0.22 | 2,287.65 |
| 87 | H-EAVTMSLSTIGSTLTSAGMT | 127 | —OH | 0.37 | 1,958.25 |
| 88 | H-GSTLTSAGMTGGPKLMIAGM | 128 | —OH | 0.37 | 1,881.28 |
| 89 | H-GGPKLMIAGMAITAITGIID | 129 | —OH | 0.54 | 1,943.42 |
| 90 | H-AITAITGIIDTIKDIYYMFS | 130 | —OH | 0.58 | 2,249.67 |
| 91 | H-TIKDIYYMFSGQERPVDPVI | 131 | —OH | 0.38 | 2,371.76 |
| 92 | H-GQERPVDPVIKLFNKYAGLM | 132 | —OH | 0.29 | 2,275.72 |
| 93 | H-KLFNKYAGLMSDNNKMGVRK | 133 | —OH | 0.04 | 2,314.78 |
| 94 | H-SDNNKMGVRKCLTPGDDTLI | 134 | —OH | 0.10 | 2,177.50 |
| 95 | H-CLTPGDDTLIYIAYRNDTSF | 135 | —OH | 0.38 | 2,278.54 |
| 96 | H-YIAYRNDTSFKQNTDAMALY | 136 | —OH | 0.19 | 2,385.66 |
| 97 | H-KQNTDAMALYFLDVIDSEIL | 137 | —OH | 0.37 | 2,299.64 |
| 98 | H-FLDVIDSEILYLNTSNLVLE | 138 | —OH | 0.53 | 2,310.64 |
| 99 | H-YLNTSNLVLEYQLKVACPIG | 139 | —OH | 0.50 | 2,238.65 |
| 100 | H-YQLKVACPIGTLRSVDVDIT | 34 | —OH | 0.42 | 2,191.59 |
| 101 | H-TLRSVDVDITAYTILYDTAD | 140 | —OH | 0.32 | 2,245.48 |
| 102 | H-AYTILYDTADNIKKYKFIRM | 141 | —OH | 0.28 | 2,467.93 |
| 103 | H-NIKKYKFIRMATLLSKHPVI | 142 | —OH | 0.37 | 2,401.02 |
| 104 | H-ATLLSKHPVIRLTCGLAATL | 143 | —OH | 0.54 | 2,078.57 |
| 105 | H-RLTCGLAATLVIKPYEVPIS | 144 | —OH | 0.54 | 2,144.62 |
| 106 | H-VIKPYEVPISDMQLLKMATP | 145 | —OH | 0.46 | 2,273.80 |
| 107 | H-DMQLLKMATPGEPESTKSIP | 146 | —OH | 0.21 | 2,173.55 |
| 108 | H-GEPESTKSIPSDVCDRYPLK | 147 | —OH | 0.09 | 2,221.49 |
| 109 | H-SDVCDRYPLKKFYLLAGGCP | 148 | —OH | 0.39 | 2,245.67 |
| 110 | H-KFYLLAGGCPYDTSQTFIVH | 149 | —OH | 0.52 | 2,260.62 |
| 111 | H-YDTSQTFIVHTTCSILLRTA | 150 | —OH | 0.47 | 2,270.61 |
| 112 | H-TTCSILLRTATRDQFRNRWV | 151 | —OH | 0.27 | 2,437.83 |
| 113 | H-TRDQFRNRWVLQNPFRQEGT | 152 | —OH | 0.04 | 2,548.82 |
| 114 | H-LQNPFRQEGTYKQLFTFSKY | 153 | —OH | 0.24 | 2,495.84 |
| 115 | H-YKQLFTFSKYDFNDTIIDPN | 154 | —OH | 0.29 | 2,469.75 |
| 116 | H-DFNDTIIDPNGVVGHASFCT | 155 | —OH | 0.34 | 2,122.31 |
| 117 | H-GVVGHASFCTNRSSNQCFWS | 156 | —OH | 0.35 | 2,187.42 |
| 118 | H-NRSSNQCFWSEPMILEDVSS | 157 | —OH | 0.26 | 2,329.57 |
| 119 | H-EPMILEDVSSCSSRTRKIYV | 158 | —OH | 0.25 | 2,313.70 |
| 120 | H-CSSRTRKIYVKLGIFNAEGF | 159 | —OH | 0.28 | 2,289.70 |
| 121 | H-KLGIFNAEGFNSFVLNCPTG | 160 | —OH | 0.43 | 2,128.45 |
| 122 | H-NSFVLNCPTGSTPTYIKHKN | 161 | —OH | 0.26 | 2,221.54 |
| 123 | H-STPTYIKHKNADSNNVIIEL | 162 | —OH | 0.18 | 2,257.54 |
| 124 | H-ADSNNVIIELPVGDYGTAKL | 163 | —OH | 0.26 | 2,089.34 |
| 125 | H-PVGDYGTAKLYSATKPSRIA | 164 | —OH | 0.18 | 2,095.40 |
| 126 | H-YSATKPSRIAVFCTHNYDKR | 165 | —OH | 0.14 | 2,357.69 |
| 127 | H-VFCTHNYDKRFKSDIIVLMF | 166 | —OH | 0.47 | 2,476.97 |
| 128 | H-FKSDIIVLMFNKNSGIPFWS | 167 | —OH | 0.55 | 2,343.79 |
| 129 | H-NKNSGIPFWSMYTGSVTSKN | 168 | —OH | 0.22 | 2,218.49 |
| 130 | H-MYTGSVTSKNRMFTTLARGM | 169 | —OH | 0.24 | 2,252.68 |
| 131 | H-RMFTTLARGMPFRSTYCDNR | 170 | —OH | 0.22 | 2,423.85 |
| 132 | H-PFRSTYCDNRRRSGCYYAGI | 35 | —OH | 0.16 | 2,385.69 |
| 133 | H-RRSGCYYAGIPFHEDSVEAD | 171 | —OH | 0.14 | 2,272.46 |
| 134 | H-PFHEDSVEADIHYGPEIMLK | 172 | —OH | 0.28 | 2,327.62 |
| 135 | H-IHYGPEIMLKETYDINSIDP | 173 | —OH | 0.35 | 2,348.68 |
| 136 | H-ETYDINSIDPRVITKSKTHF | 174 | —OH | 0.15 | 2,364.66 |
| 137 | H-RVITKSKTHETTPLSVEFMV | 175 | —OH | 0.39 | 2,316.86 |
| 138 | H-PTPLSVKFMVDNLGNGYDNP | 176 | —OH | 0.28 | 2,178.47 |
| 139 | H-DNLGNGYDNPNSFWEDAKTK | 177 | —OH | −0.05 | 2,285.38 |
| 140 | H-NSFWEDAKTKKRTYSAMTIK | 178 | —OH | 0.03 | 2,405.78 |
| 141 | H-KRTYSAMTIKVLPCTVRNKN | 36 | —OH | 0.16 | 2,323.83 |
| 142 | H-VLPCTVRNKNIDFGYNYGDI | 179 | —OH | 0.31 | 2,301.62 |
| 143 | H-IDFGYNYGDIISNMVYLQST | 180 | —OH | 0.44 | 2,313.59 |
| 144 | H-ISNMVYLQSTSQDYGDGTKY | 181 | —OH | 0.18 | 2,270.47 |
| 145 | H-SQDYGDGTKYTFKSVTRSDH | 182 | —OH | −0.07 | 2,292.42 |
| 146 | H-TFKSVTRSDHECESSLDLTS | 183 | —OH | 0.09 | 2,242.42 |
| 147 | H-ECESSLDLTSKEVTVTCPAF | 184 | —OH | 0.32 | 2,159.43 |
| 148 | H-KEVTVTCPAFSIPRNISTYE | 185 | —OH | 0.32 | 2,255.59 |
| 149 | H-SIPRNISTYEGLCFSVTTSK | 186 | —OH | 0.33 | 2,203.52 |
| 150 | H-GLCFSVTTSKDHCATGIGWL | 187 | —OH | 0.51 | 2,096.43 |
| 151 | H-DHCATGIGWLKSSGYGKEDA | 188 | —OH | 0.15 | 2,095.29 |
| 152 | H-KSSGYGKEDADKPRACFHHW | 37 | —OH | 0.01 | 2,319.56 |
| 153 | H-DKPRACFHHWNYYTLSLDYY | 189 | —OH | 0.38 | 2,592.90 |
| 154 | H-NYYTLSLDYYCSYEDIWRST | 190 | —OH | 0.39 | 2,555.78 |
| 155 | H-CSYEDIWRSTWPDYDPCKSY | 191 | —OH | 0.32 | 2,514.75 |
| 156 | H-WPDYDPCKSYIHIEYRDTWI | 192 | —OH | 0.43 | 2,600.91 |
| 157 | H-IHIEYRDTWIESNVLQQPPY | 193 | —OH | 0.38 | 2,501.80 |
| 158 | H-ESNVLQQPPYTFEFIHDNSN | 194 | —OH | 0.23 | 2,379.54 |
| 159 | H-TFEFIHDNSNEYVDKEISNK | 38 | —OH | 0.03 | 2,429.60 |
| 160 | H-EYVDKEISNKLNDLYNEYKK | 195 | —OH | −0.08 | 2,505.78 |
| 161 | H-LNDLYNEYKKIMEYSDGSLP | 196 | —OH | 0.18 | 2,392.69 |
| 162 | H-IMEYSDGSLPASINRLAKAL | 197 | —OH | 0.30 | 2,149.51 |
| 163 | H-ASINRLAKALTSEGREIASV | 198 | —OH | 0.13 | 2,086.39 |
| 164 | H-TSEGREIASVNIDGNLLDIA | 199 | —OH | 0.18 | 2,087.29 |
| 165 | H-NIDGNLLDIAYQADKEKMAD | 200 | —OH | 0.06 | 2,237.49 |
| 166 | H-YQADKEKMADIQTRINDIIR | 39 | —OH | 0.02 | 2,421.78 |
| 167 | H-IQTRINDIIRDLFIHTLSDK | 201 | —OH | 0.31 | 2,411.80 |
| 168 | H-DLFIHTLSDKDIKDIIESEE | 40 | —OH | 0.18 | 2,360.62 |
| 169 | H-DIKDIIESEEGKRCCIIDVK | 202 | —OH | 0.15 | 2,306.70 |
| 170 | H-GKRCCIIDVKNNRVKKYYSI | 41 | —OH | 0.14 | 2,400.91 |
| 171 | H-NNRVKKYYSIDNYLCGTLDD | 203 | —OH | 0.10 | 2,394,66 |
| 172 | H-DNYLCGTLDDYIYTVVEYNK | 42 | —OH | 0.30 | 2,401.65 |
| 173 | H-YIYTVVEYNKSYVLVNDTYM | 204 | —OH | 0.43 | 2,477.83 |
| 174 | H-SYVLVNDTYMSYDYLESSGV | 205 | —OH | 0.35 | 2,305.52 |
| 175 | H-SYDYLESSGVVVLSCYEMTI | 206 | —OH | 0.49 | 2,258.57 |
| 176 | H-VVLSCYEMTIISLDTKDAKD | 207 | —OH | 0.33 | 2,244.63 |
| 177 | H-ISLDTKDAKDAIEDVIVASA | 208 | —OH | 0.16 | 2,074.33 |
| 178 | H-AIEDVIVASAVAEALNDMFK | 43 | —OH | 0.33 | 2,106.44 |
| 179 | H-VAEALNDMFKEFDKNVSAII | 209 | —OH | 0.26 | 2,254.61 |
| 180 | H-EFDKNVSAIIIKEEDNYLNS | 210 | —OH | 0.10 | 2,341.57 |
| 181 | H-IKEEDNYLNSSPDIYHIIYI | 211 | —OH | 0.34 | 2,439.72 |
| 182 | H-SPDIYHIIYIIGGTILLLLV | 212 | —OH | 0.88* | 2,226.74 |
| 183 | H-IGGTILLLLVIILILAIYIA | 213 | —OH | 1.10* | 2,108.78 |
| 184 | H-IILILAIYIARNKYRTRKYE | 44 | —OH | 0.37 | 2,511.07 |
| 185 | H-RNKYRTRKYEIMKYDNMSIK | 214 | —OH | −0.10 | 2,638.13 |
| 186 | H-IMKYDNMSIKSDHHDSLETV | 215 | —OH | 0.14 | 2,363.67 |
| 187 | H-KSDHHDSLETVSMEIIDNRY | 216 | —OH | 0.05 | 2,389.60 |

This Example shows, according to particular aspects, that monkeypox based antigens can be used to provide novel assays that are specific for smallpox. Peptide #67 GSSIVSPLTRKGATRRRPRR (SEQ ID NO:31) was tested on multiple smallpox survivors and found to be a good diagnostic marker that is positive for smallpox but negative for monkeypox. Peptide #67 is recognized by 3/4 smallpox survivors as well as the smallpox international serum standard (pool of 63 blood samples), but is not recognized by monkeypox patients (0/20 samples score positive).

Therefore, according to particular aspects, particular monkeypox antigens can be used to simultaneously detect immunity against smallpox or monkeypox, and can be used to generate antibody reagents for direct detection of both smallpox and monkeypox. According to additional aspects, particular monkeypox antigens can be used to specifically detect smallpox, and can be used to generate antibody reagents for direct and specific detection of smallpox.

1. A high-throughput method for detecting monkeypox virus (MPV) infection, comprising:
obtaining a test serum sample from a test subject; and
detecting MPV in the sample using an immunologic assay based, at least in part, on use of at least one antibody reagent, or epitope-binding portion thereof, specific for an MPV protein or polypeptide antigen selected from the group consisting of D2L, N2R, N3R, B18R, B21R and epitope-bearing fragments of D2L, N2R, N3R, B18R and B21R.

2. The method of claim 1, wherein the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20.

3. The method of claim 2, wherein the MPV polypeptide antigen is selected from the group consisting of SEQ ID NOS:2-5, 7-9, 11-15, 17-19, 21-29, and epitope bearing fragments of SEQ ID NOS:2-5, 7-9, 11-15, 17-19, 21-29 and 30-44.

4. The method of claim 2, wherein the MPV protein or polypeptide antigen is selected from the group consisting of SEQ ID NOS:10, 20 and epitope-bearing fragments of SEQ ID NOS:10 and 20.

5. The method of claim 4, wherein the MPV polypeptide antigen is selected from the group consisting of SEQ ID NOS:11-15, 21-29 and epitope bearing fragments of SEQ ID NOS:11-15 and 21-29.

6. The method of claim 5, wherein the MPV polypeptide antigen is selected from the group consisting of SEQ ID NOS:15 (MPV N3R₁₅₇₋₁₇₆), 27 (MPV B21R₇₂₉₋₇₄₈), 31, and epitope-bearing fragments of SEQ ID NOS:15 and 27.

7. The method of claim 1, wherein the immunologic assay is selected from the group consisting of ELISA, immunoprecipitation, immunohistocytochemistry, Western analysis, antigen capture assays, two-antibody sandwich assays and combinations thereof.

8. The method of claim 1, wherein the antibody is selected from the group consisting of a single-chain antibody, a monoclonal antibody, a chimeric antibody, a humanized antibody, and a Fab fragment.

9. The method of claim 1, wherein a plurality of antibodies, or epitope-binding portions thereof, are used, in each case specific for an MPV protein or polypeptide antigen selected from the group consisting of D2L, N2R, N3R, B18R, B21R and epitope-bearing fragments of D2L, N2R, N3R, B18R and B21R.

10. A high-throughput method for detecting a monkeypox virus (MPV)-specific immune response, comprising:
obtaining a test serum sample from a test subject; and
detecting MPV-specific antibodies in the sample using an immunologic assay, based, at least in part, on use of at least one MPV protein or polypeptide selected from the group consisting of D2L, N2R, N3R, B18R, B21R, epitope-bearing fragments of D2L, N2R, N3R, B18R and B21R, and combinations thereof.

11. The method of claim 10, wherein the monkeypox virus (MPV) protein or polypeptide is selected from the group consisting of SEQ ID NOS:1, 6, 10, 16, 20, and epitope-bearing fragments of SEQ ID NOS:1, 6, 10, 16 and 20.

12. The method of claim 11, wherein the MPV polypeptide is selected from the group consisting of SEQ ID NOS:2-5, 7-9, 11-15, 17-19, 21-29, and epitope bearing fragments of SEQ ID NOS:2-5, 7-9, 11-15, 17-19, 21-29 and 30-44.

13. The method of claim 11, wherein the MPV protein or polypeptide is selected from the group consisting of SEQ ID NOS:10, 20 and epitope-bearing fragments of SEQ ID NOS:10 and 20.

14. The method of claim 13, wherein the MPV polypeptide is selected from the group consisting of SEQ ID NOS:11-15, 21-29 and epitope bearing fragments of SEQ ID NOS:11-15 and 21-29.

15. The method of claim 14, wherein the MPV polypeptide is selected from the group consisting of SEQ ID NOS:15 (MPV N3R₁₅₇₋₁₇₆), 27 (MPV B21R₇₂₉₋₇₄₈), and epitope-bearing fragments of SEQ ID NOS:15 and 27.

16. The method of claim 10, wherein the immunologic assay is selected from the group consisting of ELISA, immunoprecipitation, immunocytochemistry, Western analysis, antigen capture assays, two-antibody sandwich assays and combinations thereof.

17. The method of claim 10, wherein a plurality of MPV proteins or polypeptides are used, in each case selected from the group consisting of D2L, N2R, N3R, B18R, B21R and epitope-bearing fragments of D2L, N2R, N3R, B18R and B21R.

18. The method of claim 10, wherein detecting monkeypox virus (MPV)-specific antibodies in the sample further comprises determining an amount of MPV-specific antibodies in the sample, and further comprising:
determining, based at least in part on the amount of MPV-specific antibodies, a corresponding amount of MPV-neutralizing antibodies; thereby providing a determination of a level of protective immunity against MPV, based on a historic or contemporaneous correlation between amounts of MPV-neutralizing antibodies and levels of protective immunity against MPV.

19. The method of claim 18, wherein determining the amount of monkeypox virus (MPV)-neutralizing antibodies is by reference to a standard correlation between amounts of MPV-specific antibodies and amounts of MPV-neutralizing antibodies present in serum samples from previously vaccinated or infected individuals.

20. A high-throughput method for parallel detection of both monkeypox virus (MPV) infection and MPV-specific immune response, comprising:
obtaining a test serum sample from a test subject;
detecting MPV in the sample using a first immunologic assay based, at least in part, on use of at least one antibody reagent, or epitope-binding portion thereof, specific for an MPV protein or polypeptide antigen selected from the group consisting of D2L, N2R, N3R, B18R, B21R and epitope-bearing fragments of D2L, N2R, N3R, B18R and B21R; and
detecting MPV-specific antibodies in the sample using a second immunologic assay, based, at least in part, on use of at least one of the MPV proteins or polypeptides, thereby providing for detection of both monkeypox virus (MPV) infection and MPV-specific immune response using the same serum sample.

21. The method of claim 18, wherein at least one of the proteins or polypeptides used for detecting MPV-specific antibodies is the cognate antigen of one of the antibody reagents, or epitope binding portions thereof.

22. The method of claim 18, wherein the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20.

23. The method of claim 20, wherein the MPV polypeptide antigen is selected from the group consisting of SEQ ID NOS:2-5, 7-9, 11-15, 17-19, 21-29, and epitope bearing fragments of SEQ ID NOS:2-5, 7-9, 11-15, 17-19, 21-29 and 30-44.

24. The method of claim 20, wherein the MPV protein or polypeptide antigen is selected from the group consisting of SEQ ID NOS:10, 20 and epitope-bearing fragments of SEQ ID NOS:10 and 20.

25. The method of claim 22, wherein the MPV polypeptide antigen is selected from the group consisting of SEQ ID NOS:11-15, 21-29 and epitope bearing fragments of SEQ ID NOS:11-15 and 21-29.

26. The method of claim 23, wherein the MPV polypeptide antigen is selected from the group consisting of SEQ ID NOS:15 (MPV N3R₁₅₇₋₁₇₆), 27 (MPV B21R₇₂₉₋₇₄₈), and epitope-bearing fragments of SEQ ID NOS:15 and 27.

27. The method of claim 18, wherein the first and second immunologic assay is, in each case, selected from the group consisting of ELISA, immunoprecipitation, immunocytochemistry, Western analysis, antigen capture assays, two-antibody sandwich assays and combinations thereof.

28. The method of claim 18, wherein the antibody reagent is selected from the group consisting of a single-chain antibody, a monoclonal antibody, a chimeric antibody, a humanized antibody, and a Fab fragment.

29. The method of claim 18, wherein a plurality of antibody reagents, or epitope-binding portions thereof, are used, and wherein a plurality of MPV protein or polypeptide antigens are used.

30. The method of claim 27, wherein the plurality of antibody reagents, or epitope-binding portions thereof, and the plurality of MPV protein or polypeptide antigens are cognate pairs.

31. An antibody directed against a monkeypox virus (MPV) protein or polypeptide antigen selected from the group consisting of D2L, N2R, N3R, B18R, B21R and epitope-bearing fragments of D2L, N2R, N3R, B18R and B21R.

32. The antibody of claim 31, wherein the antibody is a monoclonal antibody, or antigen-binding portion thereof.

33. The antibody of claim 32, wherein the monoclonal antibody, or antigen-binding portion thereof, is a single-chain antibody, chimeric antibody, humanized antibody or Fab fragment.

34. The antibody of claim 31, wherein the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20.

35. The antibody of claim 34, wherein the monkeypox virus (MPV) polypeptide antigen is selected from the group consisting of SEQ ID NOS:2-5, 7-9, 11-15, 17-19, 21-29, and epitope bearing fragments of SEQ ID NOS:2-5, 7-9, 11-15, 17-19, 21-29 and 30-44.

36. The antibody of claim 34, wherein the monkeypox virus (MPV) protein or polypeptide antigen is selected from the group consisting of SEQ ID NOS:10, 20 and epitope-bearing fragments of SEQ ID NOS:10 and 20.

37. The antibody of claim 36, wherein the monkeypox virus (MPV) polypeptide antigen is selected from the group consisting of SEQ ID NOS:11-15, 21-29 and epitope bearing fragments of SEQ ID NOS:11-15 and 21-29.

38. The antibody of claim 37, wherein the monkeypox virus (MPV) polypeptide antigen is selected from the group consisting of SEQ ID NOS:15 (MPV N3R₁₅₇₋₁₇₆), 27 (MPV B21R₇₂₉₋₇₄₈), and epitope-bearing fragments of SEQ ID NOS:15 and 27.

39. A composition, comprising at least one antibody of claim 31.

40. The composition of claim 39, comprising a N3R-specific monoclonal antibody, and a B21R-specific monoclonal antibody.

41. The composition of claim 39, wherein at least one of the antibodies forms specific immunocomplexes with monkeypox whole virions, or proteins or polypeptides associated with monkeypox virions.

42. A pharmaceutical composition, comprising at least one antibody of claim 31, along with a pharmaceutically acceptable diluent, carrier or excipient.

43. The pharmaceutical composition of claim 42, wherein, when administered to a subject, the composition prevents or inhibits monkeypox virus infection.

44. The pharmaceutical composition of claim 42, wherein, when administered to a subject, the composition ameliorates symptoms of monkeypox virus infection.

45. The pharmaceutical composition of claim 42, wherein at least one of the antibodies forms specific immunocomplexes with monkeypox whole virions, or proteins or polypeptides associated with monkeypox virions.

46. A method of treating, or of preventing monkeypox virus infection, comprising administering to a subject in need thereof, a therapeutically effective amount of at least one antibody of claim 1, or of a pharmaceutical composition comprising the antibody.

47. An anti-monkeypox vaccine, comprising at least one monkeypox virus (MPV) protein or polypeptide selected from the group consisting of D2L, N2R, N3R, B18R, B21R and epitope-bearing fragments of D2L, N2R, N3R, B18R and B21R.

48. The composition of claim 42, wherein the immunoglobulin sequences are, or substantially are, human immunoglobulin sequences.

49. A high-throughput method for parallel detection of both virus infection and immune response against the virus, comprising:
obtaining a test serum sample from a test subject;
detecting virus in the sample using a first immunologic assay based, at least in part, on use of at least one antibody reagent, or epitope-binding portion thereof, specific for a viral protein or polypeptide antigen; and
detecting viral-specific antibodies in the sample using a second immunologic assay, based, at least in part, on use of at least one of the viral proteins or polypeptides, wherein at least one of the proteins or polypeptides used for detecting virus-specific antibodies is the cognate antigen of one of the antibody reagents, or epitope binding portions thereof.

50. The method of claim 49, wherein the first and second immunologic assay is, in each case, selected from the group consisting of ELISA, immunoprecipitation, immunocytochemistry, Western analysis, antigen capture assays, two-antibody sandwich assays and combinations thereof.

51. The method of claim 49, wherein a plurality of antibody reagents, or epitope-binding portions thereof, are used, and wherein a plurality of viral protein or polypeptide antigens are used.

52. The method of claim 51, wherein the plurality of antibody reagents, or epitope-binding portions thereof, and the plurality of viral protein or polypeptide antigens are cognate pairs.

53. The method of claim 49, wherein the virus is an orthopoxvirus.

54. The method of claim 53, wherein the orthopoxvirus is selected from the group consisting of smallpox, vaccinia and monkeypox.

55. A high-throughput method for detecting protective immunity against smallpox virus, comprising:
obtaining a test serum sample from a test subject previously vaccinated with a vaccinia-based vaccine;
detecting an amount of vaccinia virus-specific antibodies in the sample using an immunologic assay; and
determining, based at least in part on the amount of vaccinia virus-specific antibodies, a corresponding amount of vaccinia virus-neutralizing antibodies; thereby providing a determination of a level of protective immunity against smallpox virus, based on a historic correlation between amounts of vaccinia virus-neutralizing antibodies and protective immunity against small pox virus.

56. The method of claim 55, wherein determining the amount of vaccinia virus-neutralizing antibodies is by reference to a historic or contemporaneous correlation between amounts of vaccinia virus-specific antibodies and amounts of vaccinia virus-neutralizing antibodies present in serum samples from individuals previously vaccinated with a vaccinia-based vaccine.

57. The method of claim 55, wherein the vaccinia virus-neutralizing antibodies comprise vaccinia intramolecular mature virus (IMV)-neutralizing antibodies.

58. The method of claim 55, wherein the immunologic assay comprises an assay selected from the group consisting of ELISA, immunoprecipitation, immunocytochemistry, Western analysis, antigen capture assays, two-antibody sandwich assays and combinations thereof.

59. The method of claim 55, wherein detecting an amount of vaccinia virus-specific antibodies in the sample using an immunologic assay, comprises forming immunocomplexes between the vaccinia virus-specific antibodies in the sample, and treated vaccinia virus, wherein the vaccinia virus has been treated with a peroxide agent prior to immunocomplex formation.

60. The method of claim 59, wherein the peroxide-treated vaccinia virus is immobilized on a surface prior to immunocomplex formation.

61. The method of claim 59, wherein treating of the vaccinia virus with a peroxide agent comprises treating with hydrogen peroxide.

62. The method of claim 61, wherein, during the treating, the hydrogen peroxide concentration is about 0.5% to about 10%, or about 1.0% to about 5%, or about 2% to about 4%, or about 3% (vol/vol).

63. An array comprising a plurality of different monkeypox virus (MPV) proteins or polypeptides coupled to a solid phase, wherein the MPV proteins or polypeptides are selected from the group consisting of of D2L, N2R, N3R, B18R, B21R and epitope-bearing fragments of D2L, N2R, N3R, B18R and B21R.

64. The array of claim 63, wherein the monkeypox virus (MPV) protein or polypeptide antigen is selected from the group consisting of SEQ ID NOS:1, 6, 10, 16, 20, and epitope-bearing fragments of SEQ ID NOS:1, 6, 10, 16 and 20.

65. The array of claim 64, wherein the monkeypox virus (MPV) polypeptide antigen is selected from the group consisting of SEQ ID NOS:2-5, 7-9, 11-15, 17-19, 21-29, and epitope bearing fragments of SEQ ID NOS:2-5, 7-9, 11-15, 17-19, 21-29 and 30-34.

66. The array of claim 64, wherein the monkeypox virus (MPV) protein or polypeptide antigen is selected from the group consisting of SEQ ID NOS:10, 20 and epitope-bearing fragments of SEQ ID NOS:10 and 20.

67. The array of claim 66, wherein the monkeypox virus (MPV) polypeptide antigen is selected from the group consisting of SEQ ID NOS:11-15, 21-29 and epitope bearing fragments of SEQ ID NOS:11-15 and 21-29.

68. The array of claim 67, wherein the monkeypox virus (MPV) polypeptide antigen is selected from the group consisting of SEQ ID NOS:15 (MPV N3R₁₅₇₋₁₇₆), 27 (MPV B21R₇₂₉₋₇₄₈), and epitope-bearing fragments of SEQ ID NOS:15 and 27.

69. The array of claim 63, wherein the solid phase comprises a material selected from the group consisting of silicon, cellulose, glass, polystyrene, polyacrylamide, aluminium, steel, iron, copper, nickel, silver, gold and combinations thereof.

70. A high-throughput method for detecting smallpox virus infection, comprising:
obtaining a test serum sample from a test subject; and
detecting smallpox in the sample using an immunologic assay based, at least in part, on use of at least one antibody reagent, or epitope-binding portion thereof, specific for an MPV protein or polypeptide antigen selected from the group consisting of B21R and epitope-bearing fragments of B21R.

71. The method of claim 70, wherein the monkeypox virus (MPV) protein or polypeptide antigen comprises SEQ ID NO:31.

72. A high-throughput method for detecting a smallpox virus-specific immune response, comprising:
obtaining a test serum sample from a test subject; and
detecting smallpox-specific antibodies in the sample using an immunologic assay, based, at least in part, on use of at least one MPV protein or polypeptide selected from the group consisting of B21R and epitope-bearing fragments of B21R.

73. The method of claim 72, wherein the monkeypox virus (MPV) protein or polypeptide comprises SEQ ID NO:31.

74. The method of claim 49, further comprising determining an amount of virus-neutralizing antibodies corresponding to the virus-specific antibodies, thereby providing a determination of a level of protective immunity against the virus.

75. A method for systematic analysis of biologically relevant epitopes relevant to an infectious agent comprising:
(1) obtaining a test serum sample from a test subject;
(2) obtaining specific polypeptides representing sub-regions of one or more proteins relevant to the infectious agent;
(3) utilizing the specific polypeptides from step (2) for screening against positive and negative control sera; and
(4) thereby identify biologically relevant epitopes relevant to the infectious agent.

76. The method of claim 75 wherein obtaining the polypeptides of step (2) representing sub-regions of one or more proteins relevant to the infectious agent comprises studying at least a part of the genomic sequence of the infectious agent.

77. The method of claim 75 wherein the screening of step (3) comprises testing the specific polypeptides in an array or ELISA plate.

78. The method of claim 75 wherein step (4) of identifying biologically relevant epitopes relevant to the infectious agent further comprises identifying epitopes with high reactivity to positive control sera and epitopes with low reactivity to negative control sera.
